SEQUENCE LISTING (39 sequences)

SEQ ID NO: 1 - DNA, 3855 bp, Photorhabdus luminescens
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 2 - DNA, 4402 bp, Artificial Sequence
  engineered and functional indC from P. luminescens where we replaced the native T-domain with a ccdB gene which is toxic to normal E. coli cells. We used this variant to easily exchange T-domains without any background cells.
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcgcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 3 - DNA, 4004 bp, Artificial Sequence
  engineered and functional indC from P. luminescens where we replaced the native T-domain with the T-domain of the bpsA indigoidine synthetase from Streptomyces lavendulae lavendulae ATCC11924
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaagatcgatgtgaaagcactggccgcttctgaccaggtcaacgctgagctggtggaacggcccttcgtcgcacctaggaccgaaacagagaaggaaatcgcagccgtgtgggagaaagccctgagacgcgaaaatgctagtgtccaggacgatttctttgagtccggcggaaactctctgatcgccgtcggcctggtgagggaactgaatgctagactgggagtgtccctgcctctgcagagtgtcctggagtcaccaacaattgaaaagctggccgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 4 - DNA, 3995 bp, Artificial Sequence
  engineered and functional indC from P. luminescens where we replaced the native T-domain with the T-domain of the plu2642 protein from P. luminescens
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaaatcgatttcgacacattacaagtactggtcagcacagtatcacacagtccacaggtactcccaagcacctcgacagaaacacagatcgtaaagatatgggaagaagtgctaacgcgagaaagcatatctaccgaagatgacttctttgctttaggtggcaattctctgatagccgtccatctgatacaacgtttaaatgaagaatttgcgttatcgctacctctccatactctatttgaggccgcaacggttaaacaattggcagggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 5 - DNA, 3983 bp, Artificial Sequence
  engineered and functional indC from P. luminescens where we replaced the native T-domain with the T-domain of the delH4 protein from Delftia acidovorans SPH-1
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaagctggaccggcaggccctgcccgcgttcggcatgccagccgccagccaggctcccgagggcgaactggagacgctgctggcccgtatctgggccgaggtgctgggcctggagcgggtggggcgcagcgacaacttcttcgcgctgggcggtgattccatcctgggcctgcagatcgtctcgcgcctgcgccgcttcggctggaagctgtcgccacggcagctgttcgagcggcaaagcattgccgagctggcggggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 6 - DNA, 3917 bp, Artificial Sequence
  engineered and functional indC from P. luminescens where we replaced the native T-domain with a synthetic T-domain of our own design (variant 1)
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaatccgtctggcgaaaatctggatggaagttctgaaatgggactctgtttctgcgctggacgacttcttcgaatctggtggtaactctctgatggcggttgcgctggttaacaaaatcaacgcggcgttcaacatccgtctgccgctgcaaatcctgttccagtctccgaccatcgcggaactggcgcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 7 - DNA, 3917 bp, Artificial Sequence
  engineered and functional indC from P. luminescens where we replaced the native T-domain with a synthetic T-domain of our own design (variant 3)
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaatccgtctgggtaaaatctggatggaagttctgaaatgggactctgttggtgcgctggacgacttcttcgaactgggtggtcactctctgatggcggttgcgatggttaacaaaatcaacgcggcgttcaacatccgtctgccgctgcaaatcctgttccagtctccgaccatcgcggaactggcgcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 8 - DNA, 3917 bp, Artificial Sequence
  engineered and functional indC from P. luminescens where we replaced the native T-domain with a synthetic T-domain of our own design (variant 4)
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaatccgtctggcgaaaatctggatggaagttctgggttgggactctgtttctgcgctggacgacttcttcgaactgggtggtaactctctgatggcggttgcgatggttaacaaaatcaacgcggcgttcaacatccgtttcccgctgcaaatcctgttccagtctccgaccatcgcggaactggcgcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 9 - DNA, 3917 bp, Artificial Sequence
  engineered and functional indC from P. luminescens where we replaced the native T-domain with a synthetic T-domain of our own design (variant 5)
atgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaatctcgtctggcggacgtttggggtcgtgcgctgaaatacgacgacgtttctgcgcacgacgacttcttcgaatctggtggtaactctctgtctgcggtttctctgatcaacgaaatcaaccgtgcgttcggtctgaccctgccgatccaggttgttttccaggcgccgaaagttcgtgaactggcgcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 10 - DNA, 2751 bp, Artificial Sequence
  gene of unknown function from P. luminescens laumondii TT01; Pfam prediction suggests a single module NRPS with the domain sequence A-T-TE. We used the T-domain of this module to successfully engineer the indC indigoidine synthetase
atgcaatcaactctcccaataataaaatggcgcaatatattaaaaacaggacagtatcgaaaatacgatatctccagcgctcaaccggcaaatgaaaattggataacgttaaacaacattaagttaccagcgagttttcaacgaaaagagtgcctaccgggattactcttttcacacgtcagatcaactccctgggctacagcagtcattcacggtgaagagcaactcagttatttggaaatggcaattggcagtgtacatctggcctgctatctgcaaaacctgggatgtttagcgggtgattgcgtcggtatatttgttgaaccgtcgattgagcagatgatcggagtttggggaactctttttgccggtggtgcatatctgccattgtctcatgattatccagaggaacgacttcgttacatgatccacgatagcaatctgaaaatgatatttacccaagaaaaattaaaggaaaaattggtcaggttggttgcagaaaatatccatatcgtgactcttgaagacgtagagaaatcatttgaatccagtgccattaccaacaacaccctccatgactaccttagcccagataacttggcttatgtcatttatacctctggaagtacagggaaaccgaaaggtgtaatgattgagcaccgcagtatcgttaaccaaatgtgttggttaaatgaaaaatgcgatttaaatattgaaaaaacaattattcagaaaacgcccatcagctttgatgctgctcaatgggaaatattatcagtcagttgtggtagtcgggttgttattagctcatctggaacacacaggaatattccccaactcattgacctgattattcgccacaatgtgaccacgttacagtgtgttcccacgctattacaagcactgatcgataatcatcaattccgggaatgccacacccttcggcaaatattcatcggagcagagagcctatcaagaaaactcgccactcaatgtatccatacactaccaaactgtctactgattaatatgtatggcccggcggaatgtacaattaatgcttcagttttccttgttaatcactacccaatatctgacgaagttaattcagtccctattggtaagccggtatccaatacggaattttttattctcgatcaccactatcagctcgcctcagaatatgaaattggagagatttatattgcgggcactcaagtcgcaagaggatatctgaatcgtcaggatctcacagaaaagcactttcttgaaattgcaataccaccaaatacgcaaaaaatccggctttatagaaccggagacctggcttattgggataaagagggtaatgcccactttgctggtcggaaagataatcaaattaaagtgagagggatgcgggtcgaattagaagaaataaaaaatgcaatagaggttatcgatcaagtgaaacacgctgcaattttggcagaaaaagaccctcaacaccgttcgacacgattaaccgcctgtattgaattagccgatgaaacaatacgccagcaagcaaagtatgacattacttcaattctgcggagtgaacttagcaaaacattaccggactatatgttacctgacagatttttgttcctggataccatgccgctaacttccagtggaaaaatcgatttcgacacattacaagtactggtcagcacagtatcacacagtccacaggtactcccaagcacctcgacagaaacacagatcgtaaagatatgggaagaagtgctaacgcgagaaagcatatctaccgaagatgacttctttgctttaggtggcaattctctgatagccgtccatctgatacaacgtttaaatgaagaatttgcgttatcgctacctctccatactctatttgaggccgcaacggttaaacaattggcaaaaatcgttgaaggtgaagtaaccagattatcttcacgattggcctgtttacaggaaaaagatgctggattacctgtcttttgctggccaggattgggtggatacccaatgaatttacacctgctggctacacagatctgcactgatcgatcattttatggcatccaagcttacggaattaatgaaggagaggttccatactccaccatagccgaaatggtgattcaagacataacagaaatcaaaaaattacaacctactggcccatacacgctatggggatattcttttggctctgtattggctttcgaagcggcttaccaattagaacgagccggagaacatgtcgaaaaggtggttctaatcgctccagggctctcgaagataaaatatcacgttaattccacgggaacagaaaacgggtctacttaccaaaacaatgagttgatatcactattattctctgtatttgcgggcacttctcacagttcagcgttgaatgaatgcctggctaacgttatcgatgagcaaagctttgtctcttttgttcataagcactacccaactctggcccctacgttaattgttcgaattgccaggatagttattcaaacctacggacagaaatattcagcaacagaactgcaagaacgaataatcaaggcgccaattacggtgtttaatgcacgtgacgatgccgtttcttttatcgaagaagcaacaccctacctaaaacatcccccagaaaatatcaatcttaacgttgatcatttcgaggtacttaaggaatcaggtgttaacgaattagctcgattcttgagttga

SEQ ID NO: 11 - DNA, 6984 bp, Artificial Sequence
  NRPS being a synthetase of a fusion peptide consisting of Asparagine and Indigoidine
atgcagacgaacaaacaacagacgttcagcgagctgctgcaaaccgtgcaaaagcaagccctggcgtctgccacctacgatttcgcgccgctgtacgaaattcagagcacaacagtgctgaaacaggaattgatcgatcatttggtcacgtttgaaaattaccccgatcattcgatgaagcatctggaagaatcattagggtttcaattcaccgtagaaagcggagatgagcagacctcctatgatttgaacgtggtcgtcgccctcgctccctcgaacgagctgtacgtgaagctaagctacaatgccgcggtgtatgaatcgtcattcgtaaacagaatcgaagggcatctccgcaccgtcatcgaccaggtgatcggcaatccgcatgtacacctgcacgagatcggcatcatcaccgaagaggaaaagcagcaactgctcgtcgcctacaacgacacggctgctgaatatccgcgggacaaaacgattttcgagctgatcgcggaacaagcgagccggacaccagcgaaagcagcagttgtttgcggcgaggacaccctgacctatcaggagctgatggagcgttctgcccagcttgccaatgctttgcgcgaaaaaggaatcgccagcggcagcatcgtctcgattatggcggaacattcactggagctgatcgtggcgatcatggctgtcttgcggtcaggtgctgcctacttgccgattgatcccgagtacccgcaagatcgcatccagtatttgctcgatgacagccagaccacgctgctgttaacccagtcgcatctgcaaccaaacatccggtttgcaggcagcgtgctttatttggacgatcgttccttgtacgaaggcggcagcacatccttcgcacccgagagcaagcctgatgatttggcgtacatgatctacacttccggttctaccggcaatccaaaaggggcgatgattactcatcaaggcctggtcaattacatctggtgggccaacaaggtgtacgtccaaggcgaagcggtggactttccgctgtactcatctatttcgttcgatttgaccgtcacctcgatcttcacgccgcttctgtccggcaacacgattcatgtgtacagaggggcagacaaggtacaggtcattttggacatcatcaaagataacaaagtcgggatcatcaagctgacgccgacacacctgaagctgattgaacacatcgacggcaaggccagcagcatcagacggttcatcgtcggcggcgagaacttgccgacaaagctggcgaagcaaatatacgaccatttcggagagaacgtgcaaattttcaacgagtacggaccgaccgaaaccgttgtcggttgcatgatttacttgtatgacccgcaaacaacgacccaggagtcggtgccaatcggtgtcccggcagacaacgtccagctttatttgctcgatgcttccatgcagccggtgcccgtcggctcgcttggcgaaatgtacatagccggagacggcgtagccaaagggtatttcaacagaccggagctgacgaaggaaaagtttatcgacaacccgttccgtccgggaaccaaaatgtatcgaacaggcgacctggcaaaatggctgcctgatggaaacatggagtatgcaggcagaatggactatcaagtgaagattcgcggccatcggatcgagatgggcgaaatcgaaacgcgcctgacgcagcatgaggcggtcaaggaagcggtcgtgatcgtggaaaaggatgagagcggccaaaacgtgttgtacgcgtaccttgtttccgagcgggaactgacggtagctgagctgagagaatttttggggcgcacgctgccttcctatatgattccttccttctttattcgcttggcggaaattccgctgaccgcgaacggaaaagtagagcgaaaaaaattgccgaagccagctggcgcagtcgttacaggcaccgcgtatgcagctccgcaaaatgaaatcgaggcaaagctggccgagatatggcagcaagtgctgggcataagccaggtagggattcacgacgatttctttgacttgggcggacactcgttgaaggcgatgactgtcgttttccaagtctcgaaagcgctggaagtggaattgcccgtaaaggccttgttcgaacatccaaccgttgcggagctggcccgcttcctttcgcggtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 12 - DNA, 5450 bp, Artificial Sequence
  Construct that enables easy cloning of NRPS modules in front of Indigoidine module through the exchange of ccdB.
actggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccacgcgtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagagttcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccacgagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 13 - DNA, 9666 bp, Artificial Sequence
  NRPS synthesizing a Indigoidine-tagged Dipeptide consisting of Ornithine and Valine
atgctgcacagcttcctcgcaaccaaaacagcctatccgacggacaaaacgttccagaagctgttcgaggagcaagtggaaaaaacaccgaacgagattgccgttctgttcggcaatgaacagctgacctatcaggagttgaatgcaaaagcaaaccagctcgcccgcgtcctgcggcgaaaaggcgtcaagccggagagcaccgtcggcatcctcgtagaccgctcgctctacatggtcatcggcatgctggccgtgttgaaagcaggcggaacattcgtcccgattgatccggactacccgctggagcgccaagcgttcatgctcgaagacagcgaggcgaagctgctgctcaccttgcaaaaaatgaacagtcaagttgccttcccttatgaaaccttttatctggatacagagacagtggatcaggaggagacgggcaatctggagcacgttgcgcagccggagaacgtcgcttacatcatctacacatccggtacgacgggcaagccaaaaggggtcgtcatcgagcaccgcagctatgccaatgtcgcatttgcctggaaagacgaatatcacctggacagcttcccggtccgtttgctgcaaatggcgagcttcgcctttgacgtctcgacgggcgattttgccagggcgctgctgacaggcgggcaactggtcatctgcccgaatggggtcaaaatggacccagcttcgctgtacgagaccatcaggcgtcacgaaattaccattttcgaagcgacacccgccttgatcatgccgttgatgcactacgtttacgaaaacgaactggatatgagccaaatgaagctgctgattctcggagcagacagctgcccggcggaagacttcaaaacgttgctcgcgcgcttcggtcagaagatgcgcattatcaacagctacggcgtgacagaggcgtgcattgacaccagctactacgaagaaacagacgtcaccgccatccgctcgggaacggtgccgatcggcaaaccgcttccgaacatgacgatgtacgtggtcgatgcgcatttgaatttgcagcctgtcggcgtcgtaggcgaattgtgcatcggcggagcaggggttgcgcgcggttatttgaacagacctgagctgacggaagagaagttcgtgccgaatccgttcgccccaggtgaacgattgtaccgcacaggtgatctggcgaagtggcgcgcagatggcaatgtcgagttcctcggacgcaatgaccaccaggtaaaaatcaggggtgtccgcatcgagctgggcgagatcgagacacaactgcgcaagctggacggaattacggaagcagtcgtggttgcgagagaagatcgcgggcaggaaaaggaattgtgcgcatacgtcgtggcggaccacaagcttgacaccgcagaattgcgggcgaatttgctgaaggaactgccgcaagcgatgattccagcgtatttcgtcaccttggatgcgctgccgctgactgccaatggcaaagtagacagacgttccttgccagcgccggatgtcaccatgctgagaacgaccgagtatgtagcgccgcgctccgtctgggaagcccgattggcccaagtatgggagcaggtgctgaatgttccgcaagtgggtgcgctagacgactttttcgcgctcggcggtcactcattgcgtgccatgcgcgtcctttccagcatgcacaacgaataccaggtcgacatcccgctgcgcatcttgttcgaaaaaccgacgattcaggaactggcggcgttcatcgaaacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgcctatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 14 - DNA, 12771 bp, Artificial Sequence
  NRPS synthesizing a Indigoidine-tagged Tripeptide consisting of Ornithine and two Valines
atgctgcacagcttcctcgcaaccaaaacagcctatccgacggacaaaacgttccagaagctgttcgaggagcaagtggaaaaaacaccgaacgagattgccgttctgttcggcaatgaacagctgacctatcaggagttgaatgcaaaagcaaaccagctcgcccgcgtcctgcggcgaaaaggcgtcaagccggagagcaccgtcggcatcctcgtagaccgctcgctctacatggtcatcggcatgctggccgtgttgaaagcaggcggaacattcgtcccgattgatccggactacccgctggagcgccaagcgttcatgctcgaagacagcgaggcgaagctgctgctcaccttgcaaaaaatgaacagtcaagttgccttcccttatgaaaccttttatctggatacagagacagtggatcaggaggagacgggcaatctggagcacgttgcgcagccggagaacgtcgcttacatcatctacacatccggtacgacgggcaagccaaaaggggtcgtcatcgagcaccgcagctatgccaatgtcgcatttgcctggaaagacgaatatcacctggacagcttcccggtccgtttgctgcaaatggcgagcttcgcctttgacgtctcgacgggcgattttgccagggcgctgctgacaggcgggcaactggtcatctgcccgaatggggtcaaaatggacccagcttcgctgtacgagaccatcaggcgtcacgaaattaccattttcgaagcgacacccgccttgatcatgccgttgatgcactacgtttacgaaaacgaactggatatgagccaaatgaagctgctgattctcggagcagacagctgcccggcggaagacttcaaaacgttgctcgcgcgcttcggtcagaagatgcgcattatcaacagctacggcgtgacagaggcgtgcattgacaccagctactacgaagaaacagacgtcaccgccatccgctcgggaacggtgccgatcggcaaaccgcttccgaacatgacgatgtacgtggtcgatgcgcatttgaatttgcagcctgtcggcgtcgtaggcgaattgtgcatcggcggagcaggggttgcgcgcggttatttgaacagacctgagctgacggaagagaagttcgtgccgaatccgttcgccccaggtgaacgattgtaccgcacaggtgatctggcgaagtggcgcgcagatggcaatgtcgagttcctcggacgcaatgaccaccaggtaaaaatcaggggtgtccgcatcgagctgggcgagatcgagacacaactgcgcaagctggacggaattacggaagcagtcgtggttgcgagagaagatcgcgggcaggaaaaggaattgtgcgcatacgtcgtggcggaccacaagcttgacaccgcagaattgcgggcgaatttgctgaaggaactgccgcaagcgatgattccagcgtatttcgtcaccttggatgcgctgccgctgactgccaatggcaaagtagacagacgttccttgccagcgccggatgtcaccatgctgagaacgaccgagtatgtagcgccgcgctccgtctgggaagcccgattggcccaagtatgggagcaggtgctgaatgttccgcaagtgggtgcgctagacgactttttcgcgctcggcggtcactcattgcgtgccatgcgcgtcctttccagcatgcacaacgaataccaggtcgacatcccgctgcgcatcttgttcgaaaaaccgacgattcaggaactggcggcgttcatcgaaacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgccgcgtatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatattgccacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgcctatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 15 - DNA, 6585 bp, Artificial Sequence
  NRPS being a putative synthetase of a fusion peptide consisting of Phenylalanine and Indigoidine
atgttagcaaatcaggccaatctcatcgacaacaagcgggaactggagcagcatgcgctagttccatatgcacagggcaagtcgatccatcaattgttcgaggaacaagcagaggcttttccagaccgcgttgccatcgtttttgaaaacaggcggctttcgtatcaggagttgaacaggaaagccaatcaactggcaagagccttgctcgaaaaaggggtgcaaacagacagcatcgtcggtgtgatgatggagaagtccatcgaaaatgtcatcgcgattctggccgttcttaaagcaggcggagcctatgtgcccatcgacatcgaatatccccgcgatcgcatccaatatattttgcaggatagtcaaacgaaaatcgtgcttacccaaaaaagcgtcagccagctcgtgcatgacgtcgggtacagcggagaggtagttgtactcgacgaagaacagttggacgctcgcgagactgccaatctgcaccagcccagcaagcctacggatcttgcctatgtcatttacacctcaggcacgacaggcaagccaaaaggcaccatgcttgaacataaaggcatcgccaatttgcaatcctttttccaaaattcgtttggcgtcaccgagcaagacaggatcgggctttttgccagcatgtcgttcgacgcatccgtttgggaaatgttcatggctttgctgtctggcgccagcctgtacatcctttccaaacagacgatccatgatttcgctgcatttgaacactatttgagtgaaaatgaattgaccatcatcacactgccgccgacttatttgactcacctcaccccagagcgcatcacctcgctacgcatcatgattacggcaggatcagcttcctccgcacccttggtaaacaaatggaaagacaaactcaggtacataaatgcatacggcccgacggaaacgagcatttgcgcgacgatctgggaagccccgtccaatcagctctccgtgcaatcggttccgatcggcaaaccgattcaaaatacacatatttatatcgtcaatgaagacttgcagctactgccgactggcagcgaaggcgaattgtgcatcggcggagtcggcttggcaagaggctattggaatcggcccgacttgaccgcagaaaaattcgtagacaatccgttcgtaccaggcgaaaaaatgtaccgcacaggtgacttggccaaatggctgacggatggaacgatcgagtttctcggcagaatcgaccatcaggtgaaaatcagaggtcatcgcatcgagcttggcgaaatcgagtctgttttgttggcacatgaacacatcacagaggccgtggtcattgccagagaggatcaacacgcgggacagtatttgtgcgcctattatatttcgcaacaagaagcaactcctgcgcagctcagagactacgccgcccagaagcttccggcttacatgctgccatcttatttcgtcaagctggacaaaatgccgcttacgccaaatgacaagatcgaccgcaaagcgttgcccgagcctgatcttacggcaaaccaaagccaggctgcctaccatcctccgagaaccgagacagaatcgattctcgtctccatctggcaaaacgttttgggaattgaaaagatcgggattcgcgataatttttactcgctcggcggagattcgatccaagcgatccaggtcgtggctcgtctgcattcctatcaattgaagctagagacgaaagacttgctgaattacccgacgatcgagcaggttgctgagctggcccgcttcctttcgcggtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 16 - DNA, 14235 bp, Artificial Sequence
  NRPS synthesizing a Indigoidine-tagged Tripeptide consisting of Phenylalanine, Ornithine and Leucine
atgttagcaaatcaggccaatctcatcgacaacaagcgggaactggagcagcatgcgctagttccatatgcacagggcaagtcgatccatcaattgttcgaggaacaagcagaggcttttccagaccgcgttgccatcgtttttgaaaacaggcggctttcgtatcaggagttgaacaggaaagccaatcaactggcaagagccttgctcgaaaaaggggtgcaaacagacagcatcgtcggtgtgatgatggagaagtccatcgaaaatgtcatcgcgattctggccgttcttaaagcaggcggagcctatgtgcccatcgacatcgaatatccccgcgatcgcatccaatatattttgcaggatagtcaaacgaaaatcgtgcttacccaaaaaagcgtcagccagctcgtgcatgacgtcgggtacagcggagaggtagttgtactcgacgaagaacagttggacgctcgcgagactgccaatctgcaccagcccagcaagcctacggatcttgcctatgtcatttacacctcaggcacgacaggcaagccaaaaggcaccatgcttgaacataaaggcatcgccaatttgcaatcctttttccaaaattcgtttggcgtcaccgagcaagacaggatcgggctttttgccagcatgtcgttcgacgcatccgtttgggaaatgttcatggctttgctgtctggcgccagcctgtacatcctttccaaacagacgatccatgatttcgctgcatttgaacactatttgagtgaaaatgaattgaccatcatcacactgccgccgacttatttgactcacctcaccccagagcgcatcacctcgctacgcatcatgattacggcaggatcagcttcctccgcacccttggtaaacaaatggaaagacaaactcaggtacataaatgcatacggcccgacggaaacgagcatttgcgcgacgatctgggaagccccgtccaatcagctctccgtgcaatcggttccgatcggcaaaccgattcaaaatacacatatttatatcgtcaatgaagacttgcagctactgccgactggcagcgaaggcgaattgtgcatcggcggagtcggcttggcaagaggctattggaatcggcccgacttgaccgcagaaaaattcgtagacaatccgttcgtaccaggcgaaaaaatgtaccgcacaggtgacttggccaaatggctgacggatggaacgatcgagtttctcggcagaatcgaccatcaggtgaaaatcagaggtcatcgcatcgagcttggcgaaatcgagtctgttttgttggcacatgaacacatcacagaggccgtggtcattgccagagaggatcaacacgcgggacagtatttgtgcgcctattatatttcgcaacaagaagcaactcctgcgcagctcagagactacgccgcccagaagcttccggcttacatgctgccatcttatttcgtcaagctggacaaaatgccgcttacgccaaatgacaagatcgaccgcaaagcgttgcccgagcctgatcttacggcaaaccaaagccaggctgcctaccatcctccgagaaccgagacagaatcgattctcgtctccatctggcaaaacgttttgggaattgaaaagatcgggattcgcgataatttttactcgctcggcggagattcgatccaagcgatccaggtcgtggctcgtctgcattcctatcaattgaagctagagacgaaagacttgctgaattacccgacgatcgagcaggttgctctttttgtcaagagcacgacgagaaaaagcgatcagggcatcatcgctggaaacgtaccgcttacacccattcagaagtggtttttcgggaaaaactttacgaatacaggccattggaaccaatcgtctgtgctctatcgcccggaaggctttgatcctaaagtcatccaaagtgtcatggacaaaatcatcgaacaccacgacgcgctccgcatggtctatcagcacgaaaacggaaatgtcgttcagcacaaccgcggcttgggtggacaattatacgatttcttctcttataatctgaccgcgcaaccagacgtccagcaggcgatcgaagcagagacgcaacgtctgcacagcagcatgaatttgcaggaaggacctctggtgaaggttgccttatttcagacgttacatggcgatcatttgtttctcgcaattcatcatttggtcgtggatggcatttcctggcgcattttgtttgaagatttggcaaccggatacgcgcaggcacttgcagggcaagcgatcagtctgcccgaaaaaacggattcttttcaaagctggtcacaatggttgcaagaatatgcgaacgaggcggatttgctgagcgagattccgtactgggagagtctcgaatcgcaagcaaaaaatgtgtccctgccgaaagactatgaagtgaccgactgcaaacaaaagagcgtgcgaaacatgcggatacggctgcacccggaagagaccgagcagttgttgaagcacgccaatcaggcctatcaaacggaaatcaacgatctgttgttggcggcgctcggcttggcttttgcggagtggagcaagcttgcgcaaatcgtcattcatttggaggggcacgggcgcgaggacatcatcgaacaggcaaacgtggccagaacggtcggatggtttacgtcgcaatatccggtattgctcgacttgaagcaaaccgctcccttgtccgactatatcaagctcaccaaagagaatatgcggaagattcctcgtaaagggatcggttacgacatcttgaagcatgtgacacttccagaaaatcgcggttccttatccttccgcgtgcagccggaagtgacgttcaactacttgggacagtttgatgcggacatgagaacggaactgtttacccgctcaccctacagcggcggcaacacgttaggcgcagatggcaaaaacaatctgagtcctgagtcagaggtgtacaccgctttgaatataaccggattgattgaaggcggagagctcgtcctcacattctcttacagctcggagcagtatcgggaagagtccatccagcaattgagccaaagttatcaaaagcatctgcttgccatcatcgcgcattgcaccgagaaaaaagaagtagagcgaacggcgcatattgccgagagcgcattcgagcagttcgagacgatccagccagtcgagcctgccgcgttttatcccgtgtcgtttgcccaaaagcgaatgtacatcctgcatcagttcgaaggaagcgggatcagctacaacgtgccgagtgtgctggtgctggaaggcaagctcgattatgaccgctttgctgctgccatccagagcctggttaaacggcatgaatctttgcgcacctcgttccattcggtaaacggggaaccgctgcaacgagtacatccggatgtcgagctgcctgtccgccttttggaggcgacagaagatcagagcgaatcgctcatccaggagctaatccagccgtttgatctggagatagccccgttgttcagagtgaatctgatcaagcttggcgcagagcggcacttgttcttcatggatatgcaccacattatttccgatggcgtatcgcttgcggtcatcgtcgaggaaattgccagcttgtatgcaggaaaacagctttccgacctgcgcatccagtacaaagactttgctgtgtggcagaccaagctggctcagtcggatcgcttccaaaaacaggaggatttttggacccggacgtttgccggggagattcctttgctgaatctgccccatgattatccaagaccttctgtgcagagctttgacggtgacacggtcgcgcttggcaccggacatcacctgctggaacaactgcgcaagctcgctgccgagactggcacgaccttgttcatggtgctgctggctgcctaccatgtgttgctctccaagtacgccggacaggaagaaatcgtcgtcggcacaccgatcgcaggccgctcgcacgcagatgtcgagcgcattgtcgggatgttcgtcaacacgctcgctttgaaaaatacggccgctggcagcctgagcttccgcgcctttttggaagacgtgaagcaaaatgcgctccatgccttcgagcatcaagactatccgttcgagcatctggtcgagaagctgcaagtgcggcgcgatctgagcagaaacccgctgtttgatacgatgttcagcctggggcttgccgaatcagccgaaggagaagtagcggatctgaaagtgtcgccttatccggtgaacggccacatcgccaaattcgacctttccctggatgcgatggaaaaacaggatggacttcttgttcaattcagctattgcacgaagctgttcgcaaaagaaacggttgatcgactggccgcccattacgttcagcttttgcaaacaatcacagccgatcccgacatcgagctcgcccggatcagcgtgttgtccaaagcagagacggagcacatgctgcacagcttcctcgcaaccaaaacagcctatccgacggacaaaacgttccagaagctgttcgaggagcaagtggaaaaaacaccgaacgagattgccgttctgttcggcaatgaacagctgacctatcaggagttgaatgcaaaagcaaaccagctcgcccgcgtcctgcggcgaaaaggcgtcaagccggagagcaccgtcggcatcctcgtagaccgctcgctctacatggtcatcggcatgctggccgtgttgaaagcaggcggaacattcgtcccgattgatccggactacccgctggagcgccaagcgttcatgctcgaagacagcgaggcgaagctgctgctcaccttgcaaaaaatgaacagtcaagttgccttcccttatgaaaccttttatctggatacagagacagtggatcaggaggagacgggcaatctggagcacgttgcgcagccggagaacgtcgcttacatcatctacacatccggtacgacgggcaagccaaaaggggtcgtcatcgagcaccgcagctatgccaatgtcgcatttgcctggaaagacgaatatcacctggacagcttcccggtccgtttgctgcaaatggcgagcttcgcctttgacgtctcgacgggcgattttgccagggcgctgctgacaggcgggcaactggtcatctgcccgaatggggtcaaaatggacccagcttcgctgtacgagaccatcaggcgtcacgaaattaccattttcgaagcgacacccgccttgatcatgccgttgatgcactacgtttacgaaaacgaactggatatgagccaaatgaagctgctgattctcggagcagacagctgcccggcggaagacttcaaaacgttgctcgcgcgcttcggtcagaagatgcgcattatcaacagctacggcgtgacagaggcgtgcattgacaccagctactacgaagaaacagacgtcaccgccatccgctcgggaacggtgccgatcggcaaaccgcttccgaacatgacgatgtacgtggtcgatgcgcatttgaatttgcagcctgtcggcgtcgtaggcgaattgtgcatcggcggagcaggggttgcgcgcggttatttgaacagacctgagctgacggaagagaagttcgtgccgaatccgttcgccccaggtgaacgattgtaccgcacaggtgatctggcgaagtggcgcgcagatggcaatgtcgagttcctcggacgcaatgaccaccaggtaaaaatcaggggtgtccgcatcgagctgggcgagatcgagacacaactgcgcaagctggacggaattacggaagcagtcgtggttgcgagagaagatcgcgggcaggaaaaggaattgtgcgcatacgtcgtggcggaccacaagcttgacaccgcagaattgcgggcgaatttgctgaaggaactgccgcaagcgatgattccagcgtatttcgtcaccttggatgcgctgccgctgactgccaatggcaaagtagacagacgttccttgccagcgccggatgtcaccatgctgagaacgaccgagtatgtagcgccgcgctccgtctgggaagcccgattggcccaagtatgggagcaggtgctgaatgttccgcaagtgggtgcgctagacgactttttcgcgctcggcggtcactcattgcgtgccatgcgcgtcctttccagcatgcacaacgaataccaggtcgacatcccgctgcgcatcttgttcgaaaaaccgacgattcaggaactggcggcgttcatcgaagagacagccaaagggaatgtcttctcgatcgagcctgtgcaaaagcaagcgtactatccggtctcctcggcacaaaagcgcatgtacatcctcgatcaatttgagggagtcggcatcagctacaacatgccgtcgactatgctgatcgaaggcaagctggagcgaacacgggtagaagcggcgttccagcgcttgattgcgcgacatgaaagcctgcgcacttcgtttgccgtcgtcaacggagagcctgtgcaaaacattcacgaggacgttccgtttgcgcttgcctattcggaagtcacagaacaggaggcgcgcgaactcgtttcttctctcgtgcagccgttcgatctggaggtcgcaccactcatccgcgtgtcgctgctgaaaatcggcgaggatcgttacgtgctctttaccgacatgcatcacagcatttccgatggcgtatcctccggcattcttttggcagagtgggtgcagctgtaccagggtgacgttttgccggagctgcgtatccagtacaaggactttgctgtgtggcaacaagagttttcccagtcggctgccttccacaagcaggaagcgtactggttgcaaacgtttgccgatgacattcctgtgctgaacttgccgaccgatttcacccgccccagcacccaaagctttgccggggatcagtgcacgatcggcgcgggcaaagcgctcacggaaggcttgcaccagttggcgcaggcgacgggaacgactttgtacatggttttgctcgccgcgtacaacgtgctgctcgccaagtatgccgggcaggaggacatcatcgtcggcacgccgattacaggcagatcccatgccgatctcgaaccgatcgtcggcatgttcgtgaacaccttggcgatgcgaaacaaaccgcagcgcgaaaagacttttagcgagtttttgcaagaagtcaagcaaaatgcgctggatgcgtacggccatcaggattacccgtttgaagaactggtggaaaagctcgcgatcgcgcgcgatttgagccgaaatccgctgtttgacaccgtgtttacgttccaaaacagcacggaagaggtcatgacgctgcctgaatgcacgcttgcgccgtttatgacggacgaaacaggccagcacgccaagttcgacttgactttcagcgctacggaagagcgggaagaaatgacgattggcgtggagtacagcacaagcttgtttacgcgggaaacgatggaacggttcagccgccacttcctgacgattgcagcgagcatcgtgcaaaatccgcacatccgtctgggcgagatcgacatgcttttgccagaagaaaaacagcagattttggccgggttcaacgatacggcagtcagctatgcgctggacaaaacgctgcaccagctattcgaagagcaggtcgacaaaacaccggatcaggcagcgcttctctttagcgagcaatcgctgacgtacagcgaactgaacgagcgagcaaacagactggcaagggtcctgcgcgcaaaaggagtcggaccggaccgtctggtagcgatcatggcggagcgctcgccggaaatggtgatcggtattctcggtattttgaaggcaggcggcgcttatgttcccgtcgatcccggctatccgcaggagcgcattcagtacctgctcgaagatagcaacgcagccctgctgctcagccaggcgcatctgttgccgctgttggcccaggtgtcaagcgagctgccggagtgccttgatctgaacgctgaactggatgccggactgagcggctccaacctgccagctgtcaaccaaccgactgaccttgcctacgtcatctatacatccggtacgaccggcaagccgaagggtgtcatgatcccgcatcaaggaatcgtgaactgcttgcagtggagaagagacgaatacgggttcgggccgagtgacaaggcgttgcaagtgttctcctttgccttcgacggttttgtagccagcttgttcgctccgctgctcggaggggcaacgtgcgtgttgccgcaagaagcagctgccaaagacccggtcgcgctgaaaaaactgatggccgcaacggaagtcacccattactacggcgtaccgagtctgttccaggccattctcgattgctcgacgacaaccgacttcaatcagttgcgttgcgtcactttgggcggcgagaagctgcctgtgcagcttgtgcaaaaaacaaaagaaaagcatccggcaatcgagatcaacaacgagtacggcccgacggaaaacagcgtcgtcaccaccatctcgcgctcgattgaagcggggcaagcgatcacgattggccgaccgcttgcgaacgtccaagtctacattgtagatgagcagcatcacttgcagccgattggcgtggtcggtgagctgtgcatcggcggagccgggcttgccagaggctatctgaacaaaccggagctgaccgcagagaagtttgtcgcaaatccgttccgaccaggcgagcgcatgtacaaaacaggcgacttggtaaaatggcggacggatggcacgatcgagtacatcggccgcgcagacgaacaggtcaaggtgagagggtatcgcatcgagatcggcgagatcgagagcgccgtactcgcttaccagggcatcgatcaagcggtggtcgttgcgcgagacgatgacgctacggctggttcctatctttgcgcctactttgtcgcagcaacagccgtgtccgtatccggcttgagaagccatctggccaaagagctgcctgcttacatgattccgagctatttcgtcgagctggatcagctgccgctttccgccaatggaaaagtggatcgcaaagctttgccgaagccgcaacagtccgatgcgaccacgcgcgaatacgtggccccgaggaatgcgaccgaacagcaactggcagccatctggcaagaagttttgggagtagagccaatcggcatcaccgaccagttctttgaactcggaggacattccttaaaagctacgctgttgattgccaaagtgtatgagtacatgcaaatcgagctgccgctgaatctcatcttccagtatccgacgatcgaaaaggtggccgatttcatcacgtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 17 - DNA, 17334 bp, Artificial Sequence
  NRPS synthesizing a Valine-Indigoidine-tagged Tripeptide consisting of Phenylalanine, Ornithine and Leucine. Valine is here used as spacer.
atgttagcaaatcaggccaatctcatcgacaacaagcgggaactggagcagcatgcgctagttccatatgcacagggcaagtcgatccatcaattgttcgaggaacaagcagaggcttttccagaccgcgttgccatcgtttttgaaaacaggcggctttcgtatcaggagttgaacaggaaagccaatcaactggcaagagccttgctcgaaaaaggggtgcaaacagacagcatcgtcggtgtgatgatggagaagtccatcgaaaatgtcatcgcgattctggccgttcttaaagcaggcggagcctatgtgcccatcgacatcgaatatccccgcgatcgcatccaatatattttgcaggatagtcaaacgaaaatcgtgcttacccaaaaaagcgtcagccagctcgtgcatgacgtcgggtacagcggagaggtagttgtactcgacgaagaacagttggacgctcgcgagactgccaatctgcaccagcccagcaagcctacggatcttgcctatgtcatttacacctcaggcacgacaggcaagccaaaaggcaccatgcttgaacataaaggcatcgccaatttgcaatcctttttccaaaattcgtttggcgtcaccgagcaagacaggatcgggctttttgccagcatgtcgttcgacgcatccgtttgggaaatgttcatggctttgctgtctggcgccagcctgtacatcctttccaaacagacgatccatgatttcgctgcatttgaacactatttgagtgaaaatgaattgaccatcatcacactgccgccgacttatttgactcacctcaccccagagcgcatcacctcgctacgcatcatgattacggcaggatcagcttcctccgcacccttggtaaacaaatggaaagacaaactcaggtacataaatgcatacggcccgacggaaacgagcatttgcgcgacgatctgggaagccccgtccaatcagctctccgtgcaatcggttccgatcggcaaaccgattcaaaatacacatatttatatcgtcaatgaagacttgcagctactgccgactggcagcgaaggcgaattgtgcatcggcggagtcggcttggcaagaggctattggaatcggcccgacttgaccgcagaaaaattcgtagacaatccgttcgtaccaggcgaaaaaatgtaccgcacaggtgacttggccaaatggctgacggatggaacgatcgagtttctcggcagaatcgaccatcaggtgaaaatcagaggtcatcgcatcgagcttggcgaaatcgagtctgttttgttggcacatgaacacatcacagaggccgtggtcattgccagagaggatcaacacgcgggacagtatttgtgcgcctattatatttcgcaacaagaagcaactcctgcgcagctcagagactacgccgcccagaagcttccggcttacatgctgccatcttatttcgtcaagctggacaaaatgccgcttacgccaaatgacaagatcgaccgcaaagcgttgcccgagcctgatcttacggcaaaccaaagccaggctgcctaccatcctccgagaaccgagacagaatcgattctcgtctccatctggcaaaacgttttgggaattgaaaagatcgggattcgcgataatttttactcgctcggcggagattcgatccaagcgatccaggtcgtggctcgtctgcattcctatcaattgaagctagagacgaaagacttgctgaattacccgacgatcgagcaggttgctctttttgtcaagagcacgacgagaaaaagcgatcagggcatcatcgctggaaacgtaccgcttacacccattcagaagtggtttttcgggaaaaactttacgaatacaggccattggaaccaatcgtctgtgctctatcgcccggaaggctttgatcctaaagtcatccaaagtgtcatggacaaaatcatcgaacaccacgacgcgctccgcatggtctatcagcacgaaaacggaaatgtcgttcagcacaaccgcggcttgggtggacaattatacgatttcttctcttataatctgaccgcgcaaccagacgtccagcaggcgatcgaagcagagacgcaacgtctgcacagcagcatgaatttgcaggaaggacctctggtgaaggttgccttatttcagacgttacatggcgatcatttgtttctcgcaattcatcatttggtcgtggatggcatttcctggcgcattttgtttgaagatttggcaaccggatacgcgcaggcacttgcagggcaagcgatcagtctgcccgaaaaaacggattcttttcaaagctggtcacaatggttgcaagaatatgcgaacgaggcggatttgctgagcgagattccgtactgggagagtctcgaatcgcaagcaaaaaatgtgtccctgccgaaagactatgaagtgaccgactgcaaacaaaagagcgtgcgaaacatgcggatacggctgcacccggaagagaccgagcagttgttgaagcacgccaatcaggcctatcaaacggaaatcaacgatctgttgttggcggcgctcggcttggcttttgcggagtggagcaagcttgcgcaaatcgtcattcatttggaggggcacgggcgcgaggacatcatcgaacaggcaaacgtggccagaacggtcggatggtttacgtcgcaatatccggtattgctcgacttgaagcaaaccgctcccttgtccgactatatcaagctcaccaaagagaatatgcggaagattcctcgtaaagggatcggttacgacatcttgaagcatgtgacacttccagaaaatcgcggttccttatccttccgcgtgcagccggaagtgacgttcaactacttgggacagtttgatgcggacatgagaacggaactgtttacccgctcaccctacagcggcggcaacacgttaggcgcagatggcaaaaacaatctgagtcctgagtcagaggtgtacaccgctttgaatataaccggattgattgaaggcggagagctcgtcctcacattctcttacagctcggagcagtatcgggaagagtccatccagcaattgagccaaagttatcaaaagcatctgcttgccatcatcgcgcattgcaccgagaaaaaagaagtagagcgaacggcgcatattgccgagagcgcattcgagcagttcgagacgatccagccagtcgagcctgccgcgttttatcccgtgtcgtttgcccaaaagcgaatgtacatcctgcatcagttcgaaggaagcgggatcagctacaacgtgccgagtgtgctggtgctggaaggcaagctcgattatgaccgctttgctgctgccatccagagcctggttaaacggcatgaatctttgcgcacctcgttccattcggtaaacggggaaccgctgcaacgagtacatccggatgtcgagctgcctgtccgccttttggaggcgacagaagatcagagcgaatcgctcatccaggagctaatccagccgtttgatctggagatagccccgttgttcagagtgaatctgatcaagcttggcgcagagcggcacttgttcttcatggatatgcaccacattatttccgatggcgtatcgcttgcggtcatcgtcgaggaaattgccagcttgtatgcaggaaaacagctttccgacctgcgcatccagtacaaagactttgctgtgtggcagaccaagctggctcagtcggatcgcttccaaaaacaggaggatttttggacccggacgtttgccggggagattcctttgctgaatctgccccatgattatccaagaccttctgtgcagagctttgacggtgacacggtcgcgcttggcaccggacatcacctgctggaacaactgcgcaagctcgctgccgagactggcacgaccttgttcatggtgctgctggctgcctaccatgtgttgctctccaagtacgccggacaggaagaaatcgtcgtcggcacaccgatcgcaggccgctcgcacgcagatgtcgagcgcattgtcgggatgttcgtcaacacgctcgctttgaaaaatacggccgctggcagcctgagcttccgcgcctttttggaagacgtgaagcaaaatgcgctccatgccttcgagcatcaagactatccgttcgagcatctggtcgagaagctgcaagtgcggcgcgatctgagcagaaacccgctgtttgatacgatgttcagcctggggcttgccgaatcagccgaaggagaagtagcggatctgaaagtgtcgccttatccggtgaacggccacatcgccaaattcgacctttccctggatgcgatggaaaaacaggatggacttcttgttcaattcagctattgcacgaagctgttcgcaaaagaaacggttgatcgactggccgcccattacgttcagcttttgcaaacaatcacagccgatcccgacatcgagctcgcccggatcagcgtgttgtccaaagcagagacggagcacatgctgcacagcttcctcgcaaccaaaacagcctatccgacggacaaaacgttccagaagctgttcgaggagcaagtggaaaaaacaccgaacgagattgccgttctgttcggcaatgaacagctgacctatcaggagttgaatgcaaaagcaaaccagctcgcccgcgtcctgcggcgaaaaggcgtcaagccggagagcaccgtcggcatcctcgtagaccgctcgctctacatggtcatcggcatgctggccgtgttgaaagcaggcggaacattcgtcccgattgatccggactacccgctggagcgccaagcgttcatgctcgaagacagcgaggcgaagctgctgctcaccttgcaaaaaatgaacagtcaagttgccttcccttatgaaaccttttatctggatacagagacagtggatcaggaggagacgggcaatctggagcacgttgcgcagccggagaacgtcgcttacatcatctacacatccggtacgacgggcaagccaaaaggggtcgtcatcgagcaccgcagctatgccaatgtcgcatttgcctggaaagacgaatatcacctggacagcttcccggtccgtttgctgcaaatggcgagcttcgcctttgacgtctcgacgggcgattttgccagggcgctgctgacaggcgggcaactggtcatctgcccgaatggggtcaaaatggacccagcttcgctgtacgagaccatcaggcgtcacgaaattaccattttcgaagcgacacccgccttgatcatgccgttgatgcactacgtttacgaaaacgaactggatatgagccaaatgaagctgctgattctcggagcagacagctgcccggcggaagacttcaaaacgttgctcgcgcgcttcggtcagaagatgcgcattatcaacagctacggcgtgacagaggcgtgcattgacaccagctactacgaagaaacagacgtcaccgccatccgctcgggaacggtgccgatcggcaaaccgcttccgaacatgacgatgtacgtggtcgatgcgcatttgaatttgcagcctgtcggcgtcgtaggcgaattgtgcatcggcggagcaggggttgcgcgcggttatttgaacagacctgagctgacggaagagaagttcgtgccgaatccgttcgccccaggtgaacgattgtaccgcacaggtgatctggcgaagtggcgcgcagatggcaatgtcgagttcctcggacgcaatgaccaccaggtaaaaatcaggggtgtccgcatcgagctgggcgagatcgagacacaactgcgcaagctggacggaattacggaagcagtcgtggttgcgagagaagatcgcgggcaggaaaaggaattgtgcgcatacgtcgtggcggaccacaagcttgacaccgcagaattgcgggcgaatttgctgaaggaactgccgcaagcgatgattccagcgtatttcgtcaccttggatgcgctgccgctgactgccaatggcaaagtagacagacgttccttgccagcgccggatgtcaccatgctgagaacgaccgagtatgtagcgccgcgctccgtctgggaagcccgattggcccaagtatgggagcaggtgctgaatgttccgcaagtgggtgcgctagacgactttttcgcgctcggcggtcactcattgcgtgccatgcgcgtcctttccagcatgcacaacgaataccaggtcgacatcccgctgcgcatcttgttcgaaaaaccgacgattcaggaactggcggcgttcatcgaagagacagccaaagggaatgtcttctcgatcgagcctgtgcaaaagcaagcgtactatccggtctcctcggcacaaaagcgcatgtacatcctcgatcaatttgagggagtcggcatcagctacaacatgccgtcgactatgctgatcgaaggcaagctggagcgaacacgggtagaagcggcgttccagcgcttgattgcgcgacatgaaagcctgcgcacttcgtttgccgtcgtcaacggagagcctgtgcaaaacattcacgaggacgttccgtttgcgcttgcctattcggaagtcacagaacaggaggcgcgcgaactcgtttcttctctcgtgcagccgttcgatctggaggtcgcaccactcatccgcgtgtcgctgctgaaaatcggcgaggatcgttacgtgctctttaccgacatgcatcacagcatttccgatggcgtatcctccggcattcttttggcagagtgggtgcagctgtaccagggtgacgttttgccggagctgcgtatccagtacaaggactttgctgtgtggcaacaagagttttcccagtcggctgccttccacaagcaggaagcgtactggttgcaaacgtttgccgatgacattcctgtgctgaacttgccgaccgatttcacccgccccagcacccaaagctttgccggggatcagtgcacgatcggcgcgggcaaagcgctcacggaaggcttgcaccagttggcgcaggcgacgggaacgactttgtacatggttttgctcgccgcgtacaacgtgctgctcgccaagtatgccgggcaggaggacatcatcgtcggcacgccgattacaggcagatcccatgccgatctcgaaccgatcgtcggcatgttcgtgaacaccttggcgatgcgaaacaaaccgcagcgcgaaaagacttttagcgagtttttgcaagaagtcaagcaaaatgcgctggatgcgtacggccatcaggattacccgtttgaagaactggtggaaaagctcgcgatcgcgcgcgatttgagccgaaatccgctgtttgacaccgtgtttacgttccaaaacagcacggaagaggtcatgacgctgcctgaatgcacgcttgcgccgtttatgacggacgaaacaggccagcacgccaagttcgacttgactttcagcgctacggaagagcgggaagaaatgacgattggcgtggagtacagcacaagcttgtttacgcgggaaacgatggaacggttcagccgccacttcctgacgattgcagcgagcatcgtgcaaaatccgcacatccgtctgggcgagatcgacatgcttttgccagaagaaaaacagcagattttggccgggttcaacgatacggcagtcagctatgcgctggacaaaacgctgcaccagctattcgaagagcaggtcgacaaaacaccggatcaggcagcgcttctctttagcgagcaatcgctgacgtacagcgaactgaacgagcgagcaaacagactggcaagggtcctgcgcgcaaaaggagtcggaccggaccgtctggtagcgatcatggcggagcgctcgccggaaatggtgatcggtattctcggtattttgaaggcaggcggcgcttatgttcccgtcgatcccggctatccgcaggagcgcattcagtacctgctcgaagatagcaacgcagccctgctgctcagccaggcgcatctgttgccgctgttggcccaggtgtcaagcgagctgccggagtgccttgatctgaacgctgaactggatgccggactgagcggctccaacctgccagctgtcaaccaaccgactgaccttgcctacgtcatctatacatccggtacgaccggcaagccgaagggtgtcatgatcccgcatcaaggaatcgtgaactgcttgcagtggagaagagacgaatacgggttcgggccgagtgacaaggcgttgcaagtgttctcctttgccttcgacggttttgtagccagcttgttcgctccgctgctcggaggggcaacgtgcgtgttgccgcaagaagcagctgccaaagacccggtcgcgctgaaaaaactgatggccgcaacggaagtcacccattactacggcgtaccgagtctgttccaggccattctcgattgctcgacgacaaccgacttcaatcagttgcgttgcgtcactttgggcggcgagaagctgcctgtgcagcttgtgcaaaaaacaaaagaaaagcatccggcaatcgagatcaacaacgagtacggcccgacggaaaacagcgtcgtcaccaccatctcgcgctcgattgaagcggggcaagcgatcacgattggccgaccgcttgcgaacgtccaagtctacattgtagatgagcagcatcacttgcagccgattggcgtggtcggtgagctgtgcatcggcggagccgggcttgccagaggctatctgaacaaaccggagctgaccgcagagaagtttgtcgcaaatccgttccgaccaggcgagcgcatgtacaaaacaggcgacttggtaaaatggcggacggatggcacgatcgagtacatcggccgcgcagacgaacaggtcaaggtgagagggtatcgcatcgagatcggcgagatcgagagcgccgtactcgcttaccagggcatcgatcaagcggtggtcgttgcgcgagacgatgacgctacggctggttcctatctttgcgcctactttgtcgcagcaacagccgtgtccgtatccggcttgagaagccatctggccaaagagctgcctgcttacatgattccgagctatttcgtcgagctggatcagctgccgctttccgccaatggaaaagtggatcgcaaagctttgccgaagccgcaacagtccgatgcgaccacgcgcgaatacgtggccccgaggaatgcgaccgaacagcaactggcagccatctggcaagaagttttgggagtagagccaatcggcatcaccgaccagttctttgaactcggaggacattccttaaaagctacgctgttgattgccaaagtgtatgagtacatgcaaatcgagctgccgctgaatctcatcttccagtatccgacgatcgaaaaggtggccgatttcatcacgacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgcctatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 18 - DNA, 9756 bp, Artificial Sequence
  NRPS synthesizing a Indigoidine-tagged Dipeptide consisting of Proline and Leucine
atggattgcgtggcaaacaattcgggagtcgagctttgccagattccgttgctgacagaagcagaaactagccagctgttggcaaagcgtacggaaacagcggctgactatcctgccgcaaccatgcacgagctgttttcgcggcaggcagaaaaaacgcctgagcaagtggcggtagtcttcgcggatcagcacctgacgtatcgggagctggatgaaaaatccaatcagctcgcccgctttttgcgcaaaaaaggcattggcacgggcagtcttgtcggcacgctgctggatcgctcgctggacatgatcgtcggaatcctcggcgtcttgaaggcaggcggcgcatttgtgccgatcgacccggagttgcctgccgaacgaatcgcttacatgctgacgcatagcagagttccattggtcgtgacgcaaaatcatttgcgggcaaaagtgaccacgcctacagaaacaattgacatcaacacagcggtgatcggggaagagagccgcgcccctatcgaatcgctcaatcagccgcatgacttgttttacatcatctatacgtccggaacgacagggcaaccgaaaggcgtcatgctggagcatcgcaacatggcgaacctgatgcattttacgtttgatcagacgaacatcgcttttcatgaaaaagtgttgcagtataccacgtgcagctttgatgtttgctaccaggaaattttctccacgctgctatccgggggccagctctacctgatcacgaacgagctgagacggcatgtggaaaagctgtttgctttcatccaggaaaagcagatcagcattttgtctctcccggtgtccttcctgaaatttatttttaacgaacaagactacgcgcaaagcttcccgcgttgtgtcaaacatatcatcacggccggggaacaactcgtcgtcacacacgagctgcaaaagtatctgcgccagcatcgcgtatttttgcacaatcactacggcccgtcggagacgcatgtggtgacgacatgcacgatggacccgggacaggcgataccagagctgccgcccatcggaaagccgatcagcaacacaggcatttacattttggatgaagggctgcaattgaagccggaggggatcgtcggggagttgtacatttccggcgcaaacgtaggaagagggtatttgcaccagccggagctgaccgcggagaagtttctcgacaatccgtatcagccaggcgaaagaatgtaccgaacgggtgatctggccctttggttgccggatggccagctcgaatttttgggccgaatcgaccatcaggtaaaaatcaggggccatcgcatcgagctgggagagatcgaatcgcgcctgctcaaccatcccgccatcaaggaagcggtggttatcgaccgagcagacgagacaggcggcaagtttttgtgcgcctatgtcgtcctgcaaaaagcgctcagcgacgaagagatgcgggcatacttggcgcaagcgttgccggagtatatgatcccttcctttttcgtgacgctggagcggattccagtcacgccgaacggaaaaacagacaggcgagctttgccgaagccggaaggaagtgccaagacgaaagcggattacgtcgccccgacgactgagctggaacaaaagctggtcgcgatttgggagcaaattcttggcgtgtcgccgatcggcattcaggatcattttttcacgctgggcggccattcgttaaaagcgattcagctcatttcccgcatccaaaaggaatgccaggcggatgtcccgctgcgcgtcctgtttgagcaaccgacgattcaagcgctggcagcgtatgtggaaggcggggaggaagggaatgtcttctcgatcgagcctgtgcaaaagcaagcgtactatccggtctcctcggcacaaaagcgcatgtacatcctcgatcaatttgagggagtcggcatcagctacaacatgccgtcgactatgctgatcgaaggcaagctggagcgaacacgggtagaagcggcgttccagcgcttgattgcgcgacatgaaagcctgcgcacttcgtttgccgtcgtcaacggagagcctgtgcaaaacattcacgaggacgttccgtttgcgcttgcctattcggaagtcacagaacaggaggcgcgcgaactcgtttcttctctcgtgcagccgttcgatctggaggtcgcaccactcatccgcgtgtcgctgctgaaaatcggcgaggatcgttacgtgctctttaccgacatgcatcacagcatttccgatggcgtatcctccggcattcttttggcagagtgggtgcagctgtaccagggtgacgttttgccggagctgcgtatccagtacaaggactttgctgtgtggcaacaagagttttcccagtcggctgccttccacaagcaggaagcgtactggttgcaaacgtttgccgatgacattcctgtgctgaacttgccgaccgatttcacccgccccagcacccaaagctttgccggggatcagtgcacgatcggcgcgggcaaagcgctcacggaaggcttgcaccagttggcgcaggcgacgggaacgactttgtacatggttttgctcgccgcgtacaacgtgctgctcgccaagtatgccgggcaggaggacatcatcgtcggcacgccgattacaggcagatcccatgccgatctcgaaccgatcgtcggcatgttcgtgaacaccttggcgatgcgaaacaaaccgcagcgcgaaaagacttttagcgagtttttgcaagaagtcaagcaaaatgcgctggatgcgtacggccatcaggattacccgtttgaagaactggtggaaaagctcgcgatcgcgcgcgatttgagccgaaatccgctgtttgacaccgtgtttacgttccaaaacagcacggaagaggtcatgacgctgcctgaatgcacgcttgcgccgtttatgacggacgaaacaggccagcacgccaagttcgacttgactttcagcgctacggaagagcgggaagaaatgacgattggcgtggagtacagcacaagcttgtttacgcgggaaacgatggaacggttcagccgccacttcctgacgattgcagcgagcatcgtgcaaaatccgcacatccgtctgggcgagatcgacatgcttttgccagaagaaaaacagcagattttggccgggttcaacgatacggcagtcagctatgcgctggacaaaacgctgcaccagctattcgaagagcaggtcgacaaaacaccggatcaggcagcgcttctctttagcgagcaatcgctgacgtacagcgaactgaacgagcgagcaaacagactggcaagggtcctgcgcgcaaaaggagtcggaccggaccgtctggtagcgatcatggcggagcgctcgccggaaatggtgatcggtattctcggtattttgaaggcaggcggcgcttatgttcccgtcgatcccggctatccgcaggagcgcattcagtacctgctcgaagatagcaacgcagccctgctgctcagccaggcgcatctgttgccgctgttggcccaggtgtcaagcgagctgccggagtgccttgatctgaacgctgaactggatgccggactgagcggctccaacctgccagctgtcaaccaaccgactgaccttgcctacgtcatctatacatccggtacgaccggcaagccgaagggtgtcatgatcccgcatcaaggaatcgtgaactgcttgcagtggagaagagacgaatacgggttcgggccgagtgacaaggcgttgcaagtgttctcctttgccttcgacggttttgtagccagcttgttcgctccgctgctcggaggggcaacgtgcgtgttgccgcaagaagcagctgccaaagacccggtcgcgctgaaaaaactgatggccgcaacggaagtcacccattactacggcgtaccgagtctgttccaggccattctcgattgctcgacgacaaccgacttcaatcagttgcgttgcgtcactttgggcggcgagaagctgcctgtgcagcttgtgcaaaaaacaaaagaaaagcatccggcaatcgagatcaacaacgagtacggcccgacggaaaacagcgtcgtcaccaccatctcgcgctcgattgaagcggggcaagcgatcacgattggccgaccgcttgcgaacgtccaagtctacattgtagatgagcagcatcacttgcagccgattggcgtggtcggtgagctgtgcatcggcggagccgggcttgccagaggctatctgaacaaaccggagctgaccgcagagaagtttgtcgcaaatccgttccgaccaggcgagcgcatgtacaaaacaggcgacttggtaaaatggcggacggatggcacgatcgagtacatcggccgcgcagacgaacaggtcaaggtgagagggtatcgcatcgagatcggcgagatcgagagcgccgtactcgcttaccagggcatcgatcaagcggtggtcgttgcgcgagacgatgacgctacggctggttcctatctttgcgcctactttgtcgcagcaacagccgtgtccgtatccggcttgagaagccatctggccaaagagctgcctgcttacatgattccgagctatttcgtcgagctggatcagctgccgctttccgccaatggaaaagtggatcgcaaagctttgccgaagccgcaacagtccgatgcgaccacgcgcgaatacgtggccccgaggaatgcgaccgaacagcaactggcagccatctggcaagaagttttgggagtagagccaatcggcatcaccgaccagttctttgaactcggaggacattccttaaaagctacgctgttgattgccaaagtgtatgagtacatgcaaatcgagctgccgctgaatctcatcttccagtatccgacgatcgaaaaggtggccgatttcatcacgtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 19 - DNA, 12855 bp, Artificial Sequence
  NRPS synthesizing a Valine-Indigoidine-tagged Dipeptide consisting of Proline and Leucine. Valine is here used as spacer.
atggattgcgtggcaaacaattcgggagtcgagctttgccagattccgttgctgacagaagcagaaactagccagctgttggcaaagcgtacggaaacagcggctgactatcctgccgcaaccatgcacgagctgttttcgcggcaggcagaaaaaacgcctgagcaagtggcggtagtcttcgcggatcagcacctgacgtatcgggagctggatgaaaaatccaatcagctcgcccgctttttgcgcaaaaaaggcattggcacgggcagtcttgtcggcacgctgctggatcgctcgctggacatgatcgtcggaatcctcggcgtcttgaaggcaggcggcgcatttgtgccgatcgacccggagttgcctgccgaacgaatcgcttacatgctgacgcatagcagagttccattggtcgtgacgcaaaatcatttgcgggcaaaagtgaccacgcctacagaaacaattgacatcaacacagcggtgatcggggaagagagccgcgcccctatcgaatcgctcaatcagccgcatgacttgttttacatcatctatacgtccggaacgacagggcaaccgaaaggcgtcatgctggagcatcgcaacatggcgaacctgatgcattttacgtttgatcagacgaacatcgcttttcatgaaaaagtgttgcagtataccacgtgcagctttgatgtttgctaccaggaaattttctccacgctgctatccgggggccagctctacctgatcacgaacgagctgagacggcatgtggaaaagctgtttgctttcatccaggaaaagcagatcagcattttgtctctcccggtgtccttcctgaaatttatttttaacgaacaagactacgcgcaaagcttcccgcgttgtgtcaaacatatcatcacggccggggaacaactcgtcgtcacacacgagctgcaaaagtatctgcgccagcatcgcgtatttttgcacaatcactacggcccgtcggagacgcatgtggtgacgacatgcacgatggacccgggacaggcgataccagagctgccgcccatcggaaagccgatcagcaacacaggcatttacattttggatgaagggctgcaattgaagccggaggggatcgtcggggagttgtacatttccggcgcaaacgtaggaagagggtatttgcaccagccggagctgaccgcggagaagtttctcgacaatccgtatcagccaggcgaaagaatgtaccgaacgggtgatctggccctttggttgccggatggccagctcgaatttttgggccgaatcgaccatcaggtaaaaatcaggggccatcgcatcgagctgggagagatcgaatcgcgcctgctcaaccatcccgccatcaaggaagcggtggttatcgaccgagcagacgagacaggcggcaagtttttgtgcgcctatgtcgtcctgcaaaaagcgctcagcgacgaagagatgcgggcatacttggcgcaagcgttgccggagtatatgatcccttcctttttcgtgacgctggagcggattccagtcacgccgaacggaaaaacagacaggcgagctttgccgaagccggaaggaagtgccaagacgaaagcggattacgtcgccccgacgactgagctggaacaaaagctggtcgcgatttgggagcaaattcttggcgtgtcgccgatcggcattcaggatcattttttcacgctgggcggccattcgttaaaagcgattcagctcatttcccgcatccaaaaggaatgccaggcggatgtcccgctgcgcgtcctgtttgagcaaccgacgattcaagcgctggcagcgtatgtggaaggcggggaggaagggaatgtcttctcgatcgagcctgtgcaaaagcaagcgtactatccggtctcctcggcacaaaagcgcatgtacatcctcgatcaatttgagggagtcggcatcagctacaacatgccgtcgactatgctgatcgaaggcaagctggagcgaacacgggtagaagcggcgttccagcgcttgattgcgcgacatgaaagcctgcgcacttcgtttgccgtcgtcaacggagagcctgtgcaaaacattcacgaggacgttccgtttgcgcttgcctattcggaagtcacagaacaggaggcgcgcgaactcgtttcttctctcgtgcagccgttcgatctggaggtcgcaccactcatccgcgtgtcgctgctgaaaatcggcgaggatcgttacgtgctctttaccgacatgcatcacagcatttccgatggcgtatcctccggcattcttttggcagagtgggtgcagctgtaccagggtgacgttttgccggagctgcgtatccagtacaaggactttgctgtgtggcaacaagagttttcccagtcggctgccttccacaagcaggaagcgtactggttgcaaacgtttgccgatgacattcctgtgctgaacttgccgaccgatttcacccgccccagcacccaaagctttgccggggatcagtgcacgatcggcgcgggcaaagcgctcacggaaggcttgcaccagttggcgcaggcgacgggaacgactttgtacatggttttgctcgccgcgtacaacgtgctgctcgccaagtatgccgggcaggaggacatcatcgtcggcacgccgattacaggcagatcccatgccgatctcgaaccgatcgtcggcatgttcgtgaacaccttggcgatgcgaaacaaaccgcagcgcgaaaagacttttagcgagtttttgcaagaagtcaagcaaaatgcgctggatgcgtacggccatcaggattacccgtttgaagaactggtggaaaagctcgcgatcgcgcgcgatttgagccgaaatccgctgtttgacaccgtgtttacgttccaaaacagcacggaagaggtcatgacgctgcctgaatgcacgcttgcgccgtttatgacggacgaaacaggccagcacgccaagttcgacttgactttcagcgctacggaagagcgggaagaaatgacgattggcgtggagtacagcacaagcttgtttacgcgggaaacgatggaacggttcagccgccacttcctgacgattgcagcgagcatcgtgcaaaatccgcacatccgtctgggcgagatcgacatgcttttgccagaagaaaaacagcagattttggccgggttcaacgatacggcagtcagctatgcgctggacaaaacgctgcaccagctattcgaagagcaggtcgacaaaacaccggatcaggcagcgcttctctttagcgagcaatcgctgacgtacagcgaactgaacgagcgagcaaacagactggcaagggtcctgcgcgcaaaaggagtcggaccggaccgtctggtagcgatcatggcggagcgctcgccggaaatggtgatcggtattctcggtattttgaaggcaggcggcgcttatgttcccgtcgatcccggctatccgcaggagcgcattcagtacctgctcgaagatagcaacgcagccctgctgctcagccaggcgcatctgttgccgctgttggcccaggtgtcaagcgagctgccggagtgccttgatctgaacgctgaactggatgccggactgagcggctccaacctgccagctgtcaaccaaccgactgaccttgcctacgtcatctatacatccggtacgaccggcaagccgaagggtgtcatgatcccgcatcaaggaatcgtgaactgcttgcagtggagaagagacgaatacgggttcgggccgagtgacaaggcgttgcaagtgttctcctttgccttcgacggttttgtagccagcttgttcgctccgctgctcggaggggcaacgtgcgtgttgccgcaagaagcagctgccaaagacccggtcgcgctgaaaaaactgatggccgcaacggaagtcacccattactacggcgtaccgagtctgttccaggccattctcgattgctcgacgacaaccgacttcaatcagttgcgttgcgtcactttgggcggcgagaagctgcctgtgcagcttgtgcaaaaaacaaaagaaaagcatccggcaatcgagatcaacaacgagtacggcccgacggaaaacagcgtcgtcaccaccatctcgcgctcgattgaagcggggcaagcgatcacgattggccgaccgcttgcgaacgtccaagtctacattgtagatgagcagcatcacttgcagccgattggcgtggtcggtgagctgtgcatcggcggagccgggcttgccagaggctatctgaacaaaccggagctgaccgcagagaagtttgtcgcaaatccgttccgaccaggcgagcgcatgtacaaaacaggcgacttggtaaaatggcggacggatggcacgatcgagtacatcggccgcgcagacgaacaggtcaaggtgagagggtatcgcatcgagatcggcgagatcgagagcgccgtactcgcttaccagggcatcgatcaagcggtggtcgttgcgcgagacgatgacgctacggctggttcctatctttgcgcctactttgtcgcagcaacagccgtgtccgtatccggcttgagaagccatctggccaaagagctgcctgcttacatgattccgagctatttcgtcgagctggatcagctgccgctttccgccaatggaaaagtggatcgcaaagctttgccgaagccgcaacagtccgatgcgaccacgcgcgaatacgtggccccgaggaatgcgaccgaacagcaactggcagccatctggcaagaagttttgggagtagagccaatcggcatcaccgaccagttctttgaactcggaggacattccttaaaagctacgctgttgattgccaaagtgtatgagtacatgcaaatcgagctgccgctgaatctcatcttccagtatccgacgatcgaaaaggtggccgatttcatcacgacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgcctatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 20 - DNA, 3267 bp, Brevibacillus parabrevis
atgttagcaaatcaggccaatctcatcgacaacaagcgggaactggagcagcatgcgctagttccatatgcacagggcaagtcgatccatcaattgttcgaggaacaagcagaggcttttccagaccgcgttgccatcgtttttgaaaacaggcggctttcgtatcaggagttgaacaggaaagccaatcaactggcaagagccttgctcgaaaaaggggtgcaaacagacagcatcgtcggtgtgatgatggagaagtccatcgaaaatgtcatcgcgattctggccgttcttaaagcaggcggagcctatgtgcccatcgacatcgaatatccccgcgatcgcatccaatatattttgcaggatagtcaaacgaaaatcgtgcttacccaaaaaagcgtcagccagctcgtgcatgacgtcgggtacagcggagaggtagttgtactcgacgaagaacagttggacgctcgcgagactgccaatctgcaccagcccagcaagcctacggatcttgcctatgtcatttacacctcaggcacgacaggcaagccaaaaggcaccatgcttgaacataaaggcatcgccaatttgcaatcctttttccaaaattcgtttggcgtcaccgagcaagacaggatcgggctttttgccagcatgtcgttcgacgcatccgtttgggaaatgttcatggctttgctgtctggcgccagcctgtacatcctttccaaacagacgatccatgatttcgctgcatttgaacactatttgagtgaaaatgaattgaccatcatcacactgccgccgacttatttgactcacctcaccccagagcgcatcacctcgctacgcatcatgattacggcaggatcagcttcctccgcacccttggtaaacaaatggaaagacaaactcaggtacataaatgcatacggcccgacggaaacgagcatttgcgcgacgatctgggaagccccgtccaatcagctctccgtgcaatcggttccgatcggcaaaccgattcaaaatacacatatttatatcgtcaatgaagacttgcagctactgccgactggcagcgaaggcgaattgtgcatcggcggagtcggcttggcaagaggctattggaatcggcccgacttgaccgcagaaaaattcgtagacaatccgttcgtaccaggcgaaaaaatgtaccgcacaggtgacttggccaaatggctgacggatggaacgatcgagtttctcggcagaatcgaccatcaggtgaaaatcagaggtcatcgcatcgagcttggcgaaatcgagtctgttttgttggcacatgaacacatcacagaggccgtggtcattgccagagaggatcaacacgcgggacagtatttgtgcgcctattatatttcgcaacaagaagcaactcctgcgcagctcagagactacgccgcccagaagcttccggcttacatgctgccatcttatttcgtcaagctggacaaaatgccgcttacgccaaatgacaagatcgaccgcaaagcgttgcccgagcctgatcttacggcaaaccaaagccaggctgcctaccatcctccgagaaccgagacagaatcgattctcgtctccatctggcaaaacgttttgggaattgaaaagatcgggattcgcgataatttttactcgctcggcggagattcgatccaagcgatccaggtcgtggctcgtctgcattcctatcaattgaagctagagacgaaagacttgctgaattacccgacgatcgagcaggttgctctttttgtcaagagcacgacgagaaaaagcgatcagggcatcatcgctggaaacgtaccgcttacacccattcagaagtggtttttcgggaaaaactttacgaatacaggccattggaaccaatcgtctgtgctctatcgcccggaaggctttgatcctaaagtcatccaaagtgtcatggacaaaatcatcgaacaccacgacgcgctccgcatggtctatcagcacgaaaacggaaatgtcgttcagcacaaccgcggcttgggtggacaattatacgatttcttctcttataatctgaccgcgcaaccagacgtccagcaggcgatcgaagcagagacgcaacgtctgcacagcagcatgaatttgcaggaaggacctctggtgaaggttgccttatttcagacgttacatggcgatcatttgtttctcgcaattcatcatttggtcgtggatggcatttcctggcgcattttgtttgaagatttggcaaccggatacgcgcaggcacttgcagggcaagcgatcagtctgcccgaaaaaacggattcttttcaaagctggtcacaatggttgcaagaatatgcgaacgaggcggatttgctgagcgagattccgtactgggagagtctcgaatcgcaagcaaaaaatgtgtccctgccgaaagactatgaagtgaccgactgcaaacaaaagagcgtgcgaaacatgcggatacggctgcacccggaagagaccgagcagttgttgaagcacgccaatcaggcctatcaaacggaaatcaacgatctgttgttggcggcgctcggcttggcttttgcggagtggagcaagcttgcgcaaatcgtcattcatttggaggggcacgggcgcgaggacatcatcgaacaggcaaacgtggccagaacggtcggatggtttacgtcgcaatatccggtattgctcgacttgaagcaaaccgctcccttgtccgactatatcaagctcaccaaagagaatatgcggaagattcctcgtaaagggatcggttacgacatcttgaagcatgtgacacttccagaaaatcgcggttccttatccttccgcgtgcagccggaagtgacgttcaactacttgggacagtttgatgcggacatgagaacggaactgtttacccgctcaccctacagcggcggcaacacgttaggcgcagatggcaaaaacaatctgagtcctgagtcagaggtgtacaccgctttgaatataaccggattgattgaaggcggagagctcgtcctcacattctcttacagctcggagcagtatcgggaagagtccatccagcaattgagccaaagttatcaaaagcatctgcttgccatcatcgcgcattgcaccgagaaaaaagaagtagagcgaacgcccagcgatttcagcgtcaaaggtctccaaatggaagaaatggacgatatcttcgaattgcttgcaaatacactgcgctaa

SEQ ID NO: 21 - DNA, 10764 bp, Brevibacillus parabrevis
atgagtgtatttagcaaagaacaagttcaggatatgtatgcgttgaccccgatgcaagaggggatgctgtttcacgccttgctcgatcaagagcacaactcgcatctggtacagatgtcgatttcgttgcagggcgatcttgacgttgggctatttacggatagcctgcatgtgctggtagagagatacgatgtattccgcacgttgtttctctatgaaaagctgaagcagcctttgcaagttgtcttgaagcaacggcctattccgatcgaattttacgacttgtctgcctgcgacgagtccgagaaacaacttcgctatacgcaatacaaaagggcggatcaggagcggacgtttcatctggcaaaagacccgttgatgcgggtcgcccttttccaaatgtcccagcacgactaccaggtcatctggagctttcatcacatcctcatggacggctggtgcttcagcattatttttgatgacttgcttgccatctacttgtccttgcaaaacaagacggcactctccctggagcccgtacagccatacagtcgctttatcaactggctggaaaaacaaaataaacaggccgctctcaactattggagcgactatctggaagcctatgaacaaaagactaccttgccgaagaaggaagctgccttcgccaaagcatttcaaccaacccaataccgcttttcgctgaaccgcaccttgaccaagcagctcgggaccatcgccagtcaaaatcaagtgacgctatcgacggtgattcaaacgatctggggagttctcctgcaaaaatacaatgcggcccatgatgtgctgttcggctctgttgtatccggacgccctacagacatcgtcggaatcgacaaaatggttggcttgtttatcaatacgattccattccgggtgcaagcgaaagctggtcaaacgttttccgagctgttgcaagctgtgcacaaaagaactttgcaatcacagccgtatgagcacgtgcctttgtacgacattcaaactcagtccgtcttgaagcaggagctgattgaccacctgctggtcatcgaaaattacccgctggtagaggctttgcagaaaaaagcattgaaccagcagatcggcttcacgattactgctgtggaaatgttcgagccgaccaattacgacttgactgtcatggtgatgccaaaagaagagcttgccttccgttttgactacaatgcggctctgtttgacgaacaggtcgtgcaaaaactggcggggcacctccaacagatcgcggattgcgtggcaaacaattcgggagtcgagctttgccagattccgttgctgacagaagcagaaactagccagctgttggcaaagcgtacggaaacagcggctgactatcctgccgcaaccatgcacgagctgttttcgcggcaggcagaaaaaacgcctgagcaagtggcggtagtcttcgcggatcagcacctgacgtatcgggagctggatgaaaaatccaatcagctcgcccgctttttgcgcaaaaaaggcattggcacgggcagtcttgtcggcacgctgctggatcgctcgctggacatgatcgtcggaatcctcggcgtcttgaaggcaggcggcgcatttgtgccgatcgacccggagttgcctgccgaacgaatcgcttacatgctgacgcatagcagagttccattggtcgtgacgcaaaatcatttgcgggcaaaagtgaccacgcctacagaaacaattgacatcaacacagcggtgatcggggaagagagccgcgcccctatcgaatcgctcaatcagccgcatgacttgttttacatcatctatacgtccggaacgacagggcaaccgaaaggcgtcatgctggagcatcgcaacatggcgaacctgatgcattttacgtttgatcagacgaacatcgcttttcatgaaaaagtgttgcagtataccacgtgcagctttgatgtttgctaccaggaaattttctccacgctgctatccgggggccagctctacctgatcacgaacgagctgagacggcatgtggaaaagctgtttgctttcatccaggaaaagcagatcagcattttgtctctcccggtgtccttcctgaaatttatttttaacgaacaagactacgcgcaaagcttcccgcgttgtgtcaaacatatcatcacggccggggaacaactcgtcgtcacacacgagctgcaaaagtatctgcgccagcatcgcgtatttttgcacaatcactacggcccgtcggagacgcatgtggtgacgacatgcacgatggacccgggacaggcgataccagagctgccgcccatcggaaagccgatcagcaacacaggcatttacattttggatgaagggctgcaattgaagccggaggggatcgtcggggagttgtacatttccggcgcaaacgtaggaagagggtatttgcaccagccggagctgaccgcggagaagtttctcgacaatccgtatcagccaggcgaaagaatgtaccgaacgggtgatctggccctttggttgccggatggccagctcgaatttttgggccgaatcgaccatcaggtaaaaatcaggggccatcgcatcgagctgggagagatcgaatcgcgcctgctcaaccatcccgccatcaaggaagcggtggttatcgaccgagcagacgagacaggcggcaagtttttgtgcgcctatgtcgtcctgcaaaaagcgctcagcgacgaagagatgcgggcatacttggcgcaagcgttgccggagtatatgatcccttcctttttcgtgacgctggagcggattccagtcacgccgaacggaaaaacagacaggcgagctttgccgaagccggaaggaagtgccaagacgaaagcggattacgtcgccccgacgactgagctggaacaaaagctggtcgcgatttgggagcaaattcttggcgtgtcgccgatcggcattcaggatcattttttcacgctgggcggccattcgttaaaagcgattcagctcatttcccgcatccaaaaggaatgccaggcggatgtcccgctgcgcgtcctgtttgagcaaccgacgattcaagcgctggcagcgtatgtggaaggcggggaggaaagcgcgtatctcgccattccccaggccgagccgcaagcgtattatcccgtatcgtctgcgcaaaaacgcatgctcatcttaaaccagctcgatccgcacagcacggtttacaacctgcctgtcgcgatgatcctcgaaggaacgctggataaagctcggctggagcacgccatttccaacctggtggctcgccatgagtcgttgcgcacgtcgtttcatacgatcaacggggagccagtttcccgcatccatgagcaaggccacttgccgattgtttacttggaaacggcggaagagcaagtgaacgaggtcattttggggttcatgcagccgtttgatctggtaacagctccgctatgccgggttggcttggtgaagctcgcagagaaccgtcacgtcctgatcatcgacatgcaccatatcatttcggacggagtctcttctcagctcatcctgaatgacttttcccgtttgtatcaaaacaaagctttgccagagcagcgcattcactataaagacttcgccgtttgggaaaaagcgtggacacaaacgaccgattaccaaaaacaggaaaaatattggctcgatcgatttgcgggcgaaatcccggttttgaacctgccgatggattacccgcggccagctgttcaaagctttgagggcgaacgttatttgttccgcacagaaaaacagttgttggaaagtttgcaggacgtagcccaaaagacaggcacgaccttgtacatggtgcttctcgcagcctatcatgtgctgctttccaaatactccgggcaggatgacgtgatgatcggcaccgtgactgccggcagggtgcacccggatacggagagcatgacggggatgttcgtcaacacgctggcgatgcgcaatcagtctgcgccgaccaaaacgttccggcaatttttgctggaggtaaaagacaacacgctggccgcttttgaacacgggcaatatccgtttgaagagcttgtcgaaaagttggcgatccagcgaaaccggagccgaaacccgctgttcgacaccttgttcattttgcaaaacatggatgccgacctgatcgagctggatggactgaccgtgacgccttatgtgccagagggggaagtcgccaagttcgatctgtcgctggaagcaagcgaaaaccaggcgggactttccttctgcttcgaattttgcaccaagctgttcgcacgcgagacgatcgagcgcatgtcgcttcattacttgcaaattttgcaggcagtcagcgcaaacacggagcaggagctggcgcaaatcgagatgctgactgcgcatgagaagcaggagctgctcgttcacttcaacgacacggccgccctgtatccagcggagagcacgctgtcgcagctgtttgaagatcaggcacagaaaactcctgagcaaaccgccgtcgtcttcggtgacaaacgactgacgtaccgcgaactgaacgagcgggccaaccagctcgcgcacactttgcgggcaaaaggcgtgcaggctgagcaaagcgtagggatcatggcgcaaagatcgttggaaatggcgatcggaattatcgctattctcaaagcgggcggggcgtatgtgccgatcgatccggattatccgaatgagcggattgcttacatgctggaagattgccgccgtctggtgctgacccagcagcagctcgccgaaaagatgaccgcaaacgtggaatgcctgtatctggatgaggagggcagctactcgcctcagacggaaaacatcgagccgatccataccgctgctgatctcgcttacatcatctacacatccggtacgacaggcaggccaaaaggcgtcatggtagagcatcggggaatcgtcaacagtgtgacgtggaacagggacgagtttgccctttctgtccgggacagtggaacgctgtcgctatcttttgccttcgatgcctttgcccttactttctttacgttgattgtatcaggctccacggtcgtcctgatgccggatcacgaagccaaagatccgatcgcgctacgcaacctgattgccgcttgggaatgcagctacgtcgttttcgtgcccagtatgttccaggcgatattggagtgcagcactccggcagacatccgctccatccaggcagtcatgctcgggggcgaaaagctgtcgccgaagcttgttcagctgtgcaaagcgatgcatccgcagatgagcgtgatgaatgcatacggcccgacggagagcagcgtcatggccacctacctgcgagatacacagccagatcagccgatcaccatcgggcggccgattgccaacaccgccatttacatcgtagaccagcaccatcaactgctgcctgtcggggtggtaggggaaatctgcatcggcggtcacggcttggcgcggggctattggaaaaagccggagcttactgcggagaaattcgtggccaatccagctgttccgggagagcgcatgtacaaaacaggcgatctgggcagatggctccacgacggcacgattgattttataggccgcgtcgatgaccaaatcaaggtgagaggataccggattgaggtcggggagattgaagcggttttgctcgcttacgatcagacgaatgaagctatcgtcgtcgcttatcaggacgatcgcggcgattcctatctggctgcgtatgtcacgggaaaaacggcgatagaggaatccgagcttcgcgcgcatctgttgcgagagcttccggcctacatggtgccgacctacctgattcaactggacgctttcccgctcacgccaaacggcaaggtcgaccgcaaggcactgcccaagccggaaggaaagcctgcaacaggagcagcttatgtcgcacccgctacagaagtggaggcgaagctggtcgccatttgggagaatgcgctggggatttccggcgtcggggtgttggatcacttttttgagctgggcggtcattccttgaaagcgatgacggttgtggcgcaagtgcatcgcgagtttcaaatcgaccttttgctgaagcagttttttgcagcgccaaccatccgggacttggcccgcttgatcgaacatagcgaacaggcagccggcgccgccattcaaccggcagagccgcaagcgtattatccggtatcttctgctcagcagcggatgtacttgctccatcagcttgaaggtgccggaatcagctacaacacaccgggcatcatcatgctggaaggcaagctcgatcgcgagcaattggcgaatgcgctgcaagcgttggtagatcgtcacgatattttgcggacgtcttttgagatggtcggagacgagctggtgcaaaaaattcatgaccgcgtggccgtgaacatggagtatgtgacggcagaagagcagcagatcgatgaccttttccacgcgttcgtccgtccgtttgatctttctgtgccgccattgctccgcatgagcctggtgaaactcgcggatgagcgtcacctgctcctgtacgacatgcaccatattgctgccgatgccgcatcgatcacgatcctgttcgatgaactggctgaattgtaccagggaagagaactgccggaaatgcgcatccagtacaaagattttgctgtctggcaaaaagccttgcatgagtcggatgccttcaagcagcaggaagcgtattggctgagcacgttcgctggaaatatcaccgctgtcgatgtgccgacagattttccgcgcccagccgtgaaaagttttgcaggggggcaagtcaccctgtccatggaccaagagctgctcagtgctttgcacgagttggctgcgcatacgaatacgacgctgtttatggttttgctggccgcctacaacgtgctgctcgcaaaatacgctgggcaagacgacatcatcgtgggaacgccgatctccggcaggtcacgcgccgagcttgcgcctgtcgtcggcatgttcgtccatacgctggcgatccgcaacaaaccgaccgccgagaagacattcaagcagtttttgcaggaggtcaagcaaaacgcgctcgatgctttcgaccaccaggactacccgtttgaaagccttgtggaaaagctgggcattccgcgcgatccggggcgcaatccgctgtttgacaccatgttcatcctgcaaaacgatgagttgcacgcaaaaacgctggatcagctcgtctatcgcccttatgaatcggacagcgcgcttgacgtggcgaaattcgacttgtcgttccatctgaccgagcgggaaaccgacctgttcttgcgcctggaatactgcaccaagctgttcaagcaacaaacggtagaacgaatggcgcaccacttcttgcaaattttgcgagcggtcacggccaatccggaaaatgaattgcaagagatcgagatgctgacagcagcagaaaagcaaatgctgctggtggcgttcaacgatacgcacagagaataccgggcagatcaaacaatccagcaactttttgaagagctggcggaaaaaatgcctgagcacacggcgctcgtattcgaagaaaagcgcatgtcgttccgggagctgaatgaaagagcgaaccagctcgcagccgttttgcgggaaaaaggagtcgggccagcgcagatcgtcgctttgctggtagagcgttccgccgagatggtcattgccacgcttgccacgttaaaagcgggcggcgcctttttgcccgtcgatcctgattatccggaagagcgaatccgctacatgctggaggacagccaggcaaaactggtggtgacccatgcgcacttgctgcacaaagtgagcagtcagtccgaagtcgttgatgtggatgaccctggaagctacgcaacacagacagacaacctgccgtgcgcaaacacaccgtctgatttggcttatatcatttacacgtccggtacgacgggcaagccaaaaggcgtcatgctggagcacaaaggggtagcgaatctgcaagcggtatttgcccatcatctaggcgtcacgccgcaagatcgggcagggcattttgccagcatctcgtttgacgcatcggtgtgggatatgtttggcccgttgctgtcgggagcgaccttgtacgtcttgtcccgagacgtcatcaacgattttcaacgattcgccgaatacgttcgcgataacgcgatcaccttcctcactttgccgccgacgtacgcgatttatctggagccggagcaggtgccgtcgttacgcaccctgattacagccggatcggcttcctccgttgcattggtggataaatggaaagaaaaagtcacctatgtcaatggatacggcccaacagagagcaccgtttgcgcgacactgtggaaagccaaaccggatgagccagtcgaaacgatcacgattggcaaaccgattcagaacaccaagctgtacatcgtggatgaccagttgcagttgaaagcgccggggcagatgggagaactgtgcatcagcggcttgtcgctggcgagaggctattggaatcgtccagagctgaccgccgagaagttcgtcgacaacccgtttgtgccaggaacaaagatgtaccggacaggcgacctggcaagatggctgccagatggaactatcgagtatctgggcagaatcgatcaccaagtgaaaattcgcggacatcgtgtggaactcggcgaagtggaaagcgtgctgctgcggtatgacacggtcaaagaggcagctgccatcacacatgaggacgaccgcggccaagcttacttgtgcgcctactacgtagcggagggagaagccacgcctgcgcaacttcgagcctatatggaaaacgagttgccgaactacatggttcccgccttctttatccagttggaaaagatgccgctgacaccgaatgacaagattgaccgaaaagcgctgccgaagccgaaccaggaggagaaccggactgagcaatatgcagcgccgcaaaccgagctggaacagttgctggctggcatctgggcagatgtactggggatcaagcaagtcgggacgcaagacaacttctttgaattgggcggcgattcgattaaagcgatccaggtatccacccgcctgaatgcgtcaggctggacgcttgcgatgaaagaactgttccagtacccgacgattgaagaagctgctctgcgcgtcatcccgaacagccgagagagcgagcagggtgtcgtagaaggcgagattgccttgacaccgatccagaaatggttcttcgcgaacaacttcacggatcgtcaccattggaatcaggctgtcatgctgtttcgcgaggacggctttgacgagggactcgtgcggcaagcgttccagcaaatcgtcgagcatcacgatgcgctgcgcatggtctacaagcaagaggacggggcgatcaagcaaatcaaccgcgggctgaccgacgagcgcttccgtttctactcttatgacttgaaaaatcacgcgaacagcgaagctcgcatcctggagctgtctgatcagatccagagcagcatcgatttggagcacggcccactcgttcacgtggctctgttcgccacaaaagacggggatcatttgctggtcgcgatccaccatcttgtcgtggatggcgtctcctggcgcattttgttcgaagatttttcctcagcctactcgcaggctctccatcagcaggagatcgtcttgccgaaaaagacggactccttcaaagactgggcggctcaattgcaaaagtacgcggacagcgacgagctgttgcgggaagtggcatattggcacaacttggagactacaacgacgactgcggcactgccaacagattttgtcacggcagatcgcaagcaaaaacatacgcggacactgtcgttcgcgttgacagtcccgcagacagaaaaccttttgcgtcacgttcatcatgcctatcacacagagatgaacgacctgctgctgacagcgctcggcttggccgtaaaagactgggcacatacgaatggcgtcgtcatcaatctggaaggccatgggcgcgaagacatccagaacgaaatgaacgtcacgcgcacgattggctggttcacttcgcaatatccggtggtgctcgacatggaaaaagccgaggacttgccgtaccagatcaagcaaaccaaagaaaacttgcgacggattccgaaaaaagggatcggctacgagattttgcgcacgctgacgacaagccagttgcagccgccattagcctttacgctgcggccggaaatcagctttaactatctcggtcaattcgagtcggacggaaaaacaggcgggtttacattctcgccgctcggaacagggcagttgttcagcccggaatcggagcgagtgttcctgctggacatttccgccatgatcgaggacggcgagctgcggatcagcgtggggtacagccgtctccaatatgaggaaaaaacgattgccagcctggcagacagctaccggaagcacttgctaggcatcatcgagcattgcatggcaaaagaagaaggcgagtacaccccgagcgacctgggggatgaagagctgtccatggaggagctggaaaacatcctggaatggatttga

SEQ ID NO: 22 - DNA, 19461 bp, Brevibacillus parabrevis
atgaaaaagcaggaaaacatcgcaaaaatttacccgctaaccccattgcaagagggtatgttgtttcacgctgtcacagacacgggcagcagcgcctattgcctccagatgtctgcaacgatcgagggcgattttcacctgccgctttttgaaaagagtctgaacaagctcgtggaaaactacgaagtattgcgcacggcttttgtataccaaaacatgcagcgacctcgccaagtcgtgttcaaggaaagaaaagtgaccgttccttgcgaaaacatcgcgcatttgccaagcgcagagcaggacgcgtacatacaagcgtacacgaagcaacatcatgcattcgacctgacaaaagacaacttgatgaaagcagccatttttcaaacggccgagaacaagtaccgattggtttgggccttccatcatattatcgtggacggttggacattgggcgtcttgctgcataagctgctgacctattacgcagcgctgcgaaaaggcgagccgattccgcgggaagcgacgaagccgtacagcgaatatatcaagtggctggataagcaaaacaaggacgaggccctcgcttattggcaaaactacctggcagggtatgaccatcaggctgcttttccgaaaaagaagcttggaacggaagcaagccgctatgaacatgtcgaggcgatgttcacgatcgctcccgagaagacgcagcagctgatccagatcgcgaaccaaaatcaggcgacgatgagcagcgtgtttcaagctctttggggcattttggccagcacatacaaaaatgcggacgatgtcgttttcggctcggtcgtatcaggccgcccgccgcaaatccaaggaattgagagcatggtcggcttgttcatcaacacgattccgacccgcgtccagacgaacaaacaacagacgttcagcgagctgctgcaaaccgtgcaaaagcaagccctggcgtctgccacctacgatttcgcgccgctgtacgaaattcagagcacaacagtgctgaaacaggaattgatcgatcatttggtcacgtttgaaaattaccccgatcattcgatgaagcatctggaagaatcattagggtttcaattcaccgtagaaagcggagatgagcagacctcctatgatttgaacgtggtcgtcgccctcgctccctcgaacgagctgtacgtgaagctaagctacaatgccgcggtgtatgaatcgtcattcgtaaacagaatcgaagggcatctccgcaccgtcatcgaccaggtgatcggcaatccgcatgtacacctgcacgagatcggcatcatcaccgaagaggaaaagcagcaactgctcgtcgcctacaacgacacggctgctgaatatccgcgggacaaaacgattttcgagctgatcgcggaacaagcgagccggacaccagcgaaagcagcagttgtttgcggcgaggacaccctgacctatcaggagctgatggagcgttctgcccagcttgccaatgctttgcgcgaaaaaggaatcgccagcggcagcatcgtctcgattatggcggaacattcactggagctgatcgtggcgatcatggctgtcttgcggtcaggtgctgcctacttgccgattgatcccgagtacccgcaagatcgcatccagtatttgctcgatgacagccagaccacgctgctgttaacccagtcgcatctgcaaccaaacatccggtttgcaggcagcgtgctttatttggacgatcgttccttgtacgaaggcggcagcacatccttcgcacccgagagcaagcctgatgatttggcgtacatgatctacacttccggttctaccggcaatccaaaaggggcgatgattactcatcaaggcctggtcaattacatctggtgggccaacaaggtgtacgtccaaggcgaagcggtggactttccgctgtactcatctatttcgttcgatttgaccgtcacctcgatcttcacgccgcttctgtccggcaacacgattcatgtgtacagaggggcagacaaggtacaggtcattttggacatcatcaaagataacaaagtcgggatcatcaagctgacgccgacacacctgaagctgattgaacacatcgacggcaaggccagcagcatcagacggttcatcgtcggcggcgagaacttgccgacaaagctggcgaagcaaatatacgaccatttcggagagaacgtgcaaattttcaacgagtacggaccgaccgaaaccgttgtcggttgcatgatttacttgtatgacccgcaaacaacgacccaggagtcggtgccaatcggtgtcccggcagacaacgtccagctttatttgctcgatgcttccatgcagccggtgcccgtcggctcgcttggcgaaatgtacatagccggagacggcgtagccaaagggtatttcaacagaccggagctgacgaaggaaaagtttatcgacaacccgttccgtccgggaaccaaaatgtatcgaacaggcgacctggcaaaatggctgcctgatggaaacatggagtatgcaggcagaatggactatcaagtgaagattcgcggccatcggatcgagatgggcgaaatcgaaacgcgcctgacgcagcatgaggcggtcaaggaagcggtcgtgatcgtggaaaaggatgagagcggccaaaacgtgttgtacgcgtaccttgtttccgagcgggaactgacggtagctgagctgagagaatttttggggcgcacgctgccttcctatatgattccttccttctttattcgcttggcggaaattccgctgaccgcgaacggaaaagtagagcgaaaaaaattgccgaagccagctggcgcagtcgttacaggcaccgcgtatgcagctccgcaaaatgaaatcgaggcaaagctggccgagatatggcagcaagtgctgggcataagccaggtagggattcacgacgatttctttgacttgggcggacactcgttgaaggcgatgactgtcgttttccaagtctcgaaagcgctggaagtggaattgcccgtaaaggccttgttcgaacatccaaccgttgcggagctggcccgcttcctttcgcggtcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgtttgcgcaatttttgcaggaagtcaagcaaaacgcgcttgcagcctatgaccatcaagattatccatttgaagaactcgtggaaaaactggcgattcagcgggatattagccgaaatccgttgtttgacacgttgttttctttggaaaacgcgaaccagcagtcgcttgccatcgccgagctgacagcgtcgccctatgagctgttcaacaaaatttccaagtttgatcttgctttgaacgcaagcgaatcgccagcggacattcagttccagctcacattcgcaaccaagctgttcaagaaagaaacggtcgagcgaatggcccggcattacctggaaattttgcgctggatcagtgagcagccaacggcaagcctcgcggacatcgacatgatgacggaagcggaaaaacgcacactccttctgaacgtgaacgatacgtttgtcgagcggactgccgcgaccgctttgcatcaattagtggaggagcaagcagcacgcacgcctgatgaagtggccgtcgtgtacgaagaatatgccttgacctatcgcgagctgaacgccagggcgaaccagctggcccgtttgctgcgcagtcacggaaccggaccagatacgttgatcggcattatggtggaccgttcgccaggcatggtcgtcgggatgctggctgtgctcaaagcaggcggcgcgtacacgccaatcgacccaagctatccgccagaacgaatccagtacatgctcagcgacagccaggcgccgattttgctgacgcagcgtcatttgcaggagctggctgcttatcaaggggagatcatcgacgtagacgaggaagcgatttacaccggagccgacacgaacttggacaacgtcgctggcaaagacgacttggcctatgtgatctacacatcgggatcgacgggcaatccgaaaggcgtcatgatctcccatcaggcgatttgcaatcacatgttgtggatgagagagacgttcccgctgacgaccgaggatgctgtcctgcaaaaaacgccgttcagcttcgacgcttccgtatgggagttttatttgccgctcatcaccggaggacaactggtgttggcaaagccggacgggcatcgcgacatcgcctacatgactcgtctcattcgagatgagaaaatcacgaccttgcagatggttccgtccttgctggatctggtcatgaccgacccgggctggagcgcatgcacgagcttgcagcgagtgttctgcggcggggaagcattgacgcctgccctcgtctcgcgtttttacgagacacagcaagctcagttgatcaacttgtacggccctacagagacaaccatcgatgcgacttattggccttgcccgcgccagcaggaatacagcgcaattccgatcggcaaaccgatcgacaacgtccggctgtatgtcgtcaatgccagcaaccagcttcagccagtaggcgtagcgggagagctgtgcattgccggagacggtttggcccgcggctattggcagcgcgaggagctgacgaaagcaagttttgtcgacaacccgtttgagccgggcggcaccatgtaccgtaccggagacatggtccgctatttgccagatggccatatcgagtatttgggacgcatcgaccatcaagtcaaaatcagaggtcaccgcatcgagctgggggaaatcgaagccacgcttttgcagcatgaagcggtcaaagcggtcgtcgtcatggcccgccaggatggcaaagggcaaaacagcctgtacgcctatgtcgtagcggagcaggacatccagacagcggagctgagaacgtacctgtctgccaccttgccagcctacatggttccgtccgcttttgttttcttggagcagctgccgctttcagcgaacggcaaagtggatcgcaaggcattgcctcaaccggaggatgccgccgcctctgctgccgtgtatgtggcgccgcgcaacgaatgggaagccaagctcgcagcgatatgggaaagtgtgcttggagtcgagccgatcggggttcacgatcatttctttgaactgggcggacattctttgaaagcgatgcacgtcatttctttgctccagcgcagcttccaggtggacgtaccgttgaaagtcctgtttgaatcgccaacgatcgcgggcctggccccacttgttgcggctgcccgcaaaggcacgtacacagcgatcccccctgtcgaaaagcaggagtattacccggtttccgcggcacagaagcgaatgttcattctgcagcaaatggaaggagcaggtatcagctacaacatgccaggcttcatgtatctcgacggcaagctggatacagagcggctgcaacaggcgctgaaaagtttggtgcaacgccacgaatcgttgcgcacctcgttccactccgtgcaaggcgagacggttcagcgtgtgcatgacgatgtcgatctggccatctcgtttggcgaagcgaccgaagcagagacccggcaaatagccgagcagtttatccagccgttcgatctgggaacagccccgctgttgcgtgccggactcatcaagctggcgccggaacgccacctgttcatgctcgatttgcaccatattgtcgtcgatggcgtctccatcggcctgctcatcgaggaatttgcccagctctatcacggggaagagctgccagcgctgcgcattcagtacaaagattttgccaagtggcagcaggactggttccagaccgaggaatttgccgagcaggaagcctactggctcaacacctttacgggagaaatccccgtgcttaatctgccgacggattatccaagaccgtctgtgaaaagctttgcgggagatcgcttcgtctttggctccggcactgctttgccaaaacaattgcatcagctcgcccaagagacaggcacgacgctctacatggttctgttggccgcctacaacgtgctcctgtccaaatactccaggcaagaggacatcatcgtcggcgctcctacggctggcaggtcccatgccgaaacggagtccatcgtcggaatgtttgtcaacacactggccttgcgcaacgagccagccgggggcaaaactttccgcgactttttggccgaagtgaaaatcaatacgttgggagcgtttgagcatcaagattatccgctcgatgaactcgtcgacaagctggacatgcaacgggatttgagccgcaaccctttgtttgacacggttttcattttgcaaaacatggagcaaaagccgttcgaaatggagcagttgacgattactccttattcggcagaggtgaaacaggccaagtttgacctgtcgctggaggcgtacgaagaaaacgcggaaatcatctttagcctggattacagcaccaagctgttttcgcgcgagacgatcgaaaaaatagcgacccattttatccaaatcttgcgggcggtcattgcggaaccggaaatgccgttgtccgagatcaccatgctcacagaggcggaaaagcagcgcttgctggtcgacttcaacggtgcgcacaaagattttccgcaaaacaaaacgcttcaggcgctttttgaagaacaagcggaaaagtcgccgcaggcaacagccgtggaaatcagcgggcagcccctgtcctatcaggagctgaatgagcgagccaaccagcttgccgctacgctgcgggagcggggagtacagcctgaccaacctgtagggattatggcgaaccgctctgtggagatggtcgtcggcatcctcgccatcttgaaagcaggcggagcttacgtgccgatcgacccggaatatccggaggagcgtgtcgcctacatgctgacggattgccaagcccgcctggtgctgacgcaaaagcatctgggagcgaagcttggttccagcgtgaccgcggaatgcctgtatctcgacgacgagagcaactatggtgtgcaccgctcgaatttgcagccgatcaataccgcttccgatctggcttacatcatctacacatcgggtacgactggcaagccaaaaggggtcatggtcgagcaccggggcatcgtcaacaacgtgctgtggaagaaagcggagtaccaaatgaaggttggcgacagaagcttgctgtctctgtcctttgcctttgacgctttcgttctgtccttctttacgcctgtgctttccggggcaactgtcgtactggcggaggatgaagaagccaaggacccagtctctttgaaaaagctcatcgccgcttcgcgctgcaccttgatgacaggcgtgccgagcttgttccaggccattctggaatgcagcacgccagcggatatccgtccgctgcaaaccgtcacactcggcggagaaaaaattacggcgcagcttgttgaaaaatgcaagcagctgaatcccgatctggtcatcgtcaacgagtacggcccgacagaaagcagtgtcgtcgccacctggcagcgccttgcgggtccggatgctgccatcaccatcgggcggccgattgccaacaccagcctgtacatcgtgaaccaatatcaccagctacagccaatcggcgtggtcggggagatttgcatcggcggccgcggcttggcacgaggctattggaacaagccagcgctcacggaagagaagttcgtttcccatccgtttgcggcaggcgagcgcatgtacaagacgggcgatcttggcaagtggctcccggacggaacgattgaatacattgggcgcatcgacgaacaggtcaaagtccgaggctaccgaattgaaatcggcgagatcgagtcggctctgctggctgcggaaaagctgacagcggctgttgtggtcgtctatgaggatcagcttggccagtcggctctggcagcgtattttaccgccgacgaacagcttgatgtcacgaagctgtggtcgcatctgtcgaagcgactcccgtcgtacatgattcctgcgcattttgtgcagctcgatcagcttccgcttacgccaaacggcaaagtcgacaagaaagccttgccgaagccagaaggcaagcccgtaaccgaagcgcaatatgtcgcgccgacaaatgcggtggaaagcaagctggcagagatttgggaacgcgtgctcggggttagcggcatcggcattctcgacaactttttccagatcggcggacattccttgaaagcgatggctgtcgctgcacaggtgcatcgcgagtatcaggttgagcttccgctgaaagtgctgttcgcgcagcctacgatcaaggcgttggcccagtatgtcgccacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgccgcgtatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatattgccgagagcgcattcgagcagttcgagacgatccagccagtcgagcctgccgcgttttatcccgtgtcgtttgcccaaaagcgaatgtacatcctgcatcagttcgaaggaagcgggatcagctacaacgtgccgagtgtgctggtgctggaaggcaagctcgattatgaccgctttgctgctgccatccagagcctggttaaacggcatgaatctttgcgcacctcgttccattcggtaaacggggaaccgctgcaacgagtacatccggatgtcgagctgcctgtccgccttttggaggcgacagaagatcagagcgaatcgctcatccaggagctaatccagccgtttgatctggagatagccccgttgttcagagtgaatctgatcaagcttggcgcagagcggcacttgttcttcatggatatgcaccacattatttccgatggcgtatcgcttgcggtcatcgtcgaggaaattgccagcttgtatgcaggaaaacagctttccgacctgcgcatccagtacaaagactttgctgtgtggcagaccaagctggctcagtcggatcgcttccaaaaacaggaggatttttggacccggacgtttgccggggagattcctttgctgaatctgccccatgattatccaagaccttctgtgcagagctttgacggtgacacggtcgcgcttggcaccggacatcacctgctggaacaactgcgcaagctcgctgccgagactggcacgaccttgttcatggtgctgctggctgcctaccatgtgttgctctccaagtacgccggacaggaagaaatcgtcgtcggcacaccgatcgcaggccgctcgcacgcagatgtcgagcgcattgtcgggatgttcgtcaacacgctcgctttgaaaaatacggccgctggcagcctgagcttccgcgcctttttggaagacgtgaagcaaaatgcgctccatgccttcgagcatcaagactatccgttcgagcatctggtcgagaagctgcaagtgcggcgcgatctgagcagaaacccgctgtttgatacgatgttcagcctggggcttgccgaatcagccgaaggagaagtagcggatctgaaagtgtcgccttatccggtgaacggccacatcgccaaattcgacctttccctggatgcgatggaaaaacaggatggacttcttgttcaattcagctattgcacgaagctgttcgcaaaagaaacggttgatcgactggccgcccattacgttcagcttttgcaaacaatcacagccgatcccgacatcgagctcgcccggatcagcgtgttgtccaaagcagagacggagcacatgctgcacagcttcctcgcaaccaaaacagcctatccgacggacaaaacgttccagaagctgttcgaggagcaagtggaaaaaacaccgaacgagattgccgttctgttcggcaatgaacagctgacctatcaggagttgaatgcaaaagcaaaccagctcgcccgcgtcctgcggcgaaaaggcgtcaagccggagagcaccgtcggcatcctcgtagaccgctcgctctacatggtcatcggcatgctggccgtgttgaaagcaggcggaacattcgtcccgattgatccggactacccgctggagcgccaagcgttcatgctcgaagacagcgaggcgaagctgctgctcaccttgcaaaaaatgaacagtcaagttgccttcccttatgaaaccttttatctggatacagagacagtggatcaggaggagacgggcaatctggagcacgttgcgcagccggagaacgtcgcttacatcatctacacatccggtacgacgggcaagccaaaaggggtcgtcatcgagcaccgcagctatgccaatgtcgcatttgcctggaaagacgaatatcacctggacagcttcccggtccgtttgctgcaaatggcgagcttcgcctttgacgtctcgacgggcgattttgccagggcgctgctgacaggcgggcaactggtcatctgcccgaatggggtcaaaatggacccagcttcgctgtacgagaccatcaggcgtcacgaaattaccattttcgaagcgacacccgccttgatcatgccgttgatgcactacgtttacgaaaacgaactggatatgagccaaatgaagctgctgattctcggagcagacagctgcccggcggaagacttcaaaacgttgctcgcgcgcttcggtcagaagatgcgcattatcaacagctacggcgtgacagaggcgtgcattgacaccagctactacgaagaaacagacgtcaccgccatccgctcgggaacggtgccgatcggcaaaccgcttccgaacatgacgatgtacgtggtcgatgcgcatttgaatttgcagcctgtcggcgtcgtaggcgaattgtgcatcggcggagcaggggttgcgcgcggttatttgaacagacctgagctgacggaagagaagttcgtgccgaatccgttcgccccaggtgaacgattgtaccgcacaggtgatctggcgaagtggcgcgcagatggcaatgtcgagttcctcggacgcaatgaccaccaggtaaaaatcaggggtgtccgcatcgagctgggcgagatcgagacacaactgcgcaagctggacggaattacggaagcagtcgtggttgcgagagaagatcgcgggcaggaaaaggaattgtgcgcatacgtcgtggcggaccacaagcttgacaccgcagaattgcgggcgaatttgctgaaggaactgccgcaagcgatgattccagcgtatttcgtcaccttggatgcgctgccgctgactgccaatggcaaagtagacagacgttccttgccagcgccggatgtcaccatgctgagaacgaccgagtatgtagcgccgcgctccgtctgggaagcccgattggcccaagtatgggagcaggtgctgaatgttccgcaagtgggtgcgctagacgactttttcgcgctcggcggtcactcattgcgtgccatgcgcgtcctttccagcatgcacaacgaataccaggtcgacatcccgctgcgcatcttgttcgaaaaaccgacgattcaggaactggcggcgttcatcgaagagacagccaaagggaatgtcttctcgatcgagcctgtgcaaaagcaagcgtactatccggtctcctcggcacaaaagcgcatgtacatcctcgatcaatttgagggagtcggcatcagctacaacatgccgtcgactatgctgatcgaaggcaagctggagcgaacacgggtagaagcggcgttccagcgcttgattgcgcgacatgaaagcctgcgcacttcgtttgccgtcgtcaacggagagcctgtgcaaaacattcacgaggacgttccgtttgcgcttgcctattcggaagtcacagaacaggaggcgcgcgaactcgtttcttctctcgtgcagccgttcgatctggaggtcgcaccactcatccgcgtgtcgctgctgaaaatcggcgaggatcgttacgtgctctttaccgacatgcatcacagcatttccgatggcgtatcctccggcattcttttggcagagtgggtgcagctgtaccagggtgacgttttgccggagctgcgtatccagtacaaggactttgctgtgtggcaacaagagttttcccagtcggctgccttccacaagcaggaagcgtactggttgcaaacgtttgccgatgacattcctgtgctgaacttgccgaccgatttcacccgccccagcacccaaagctttgccggggatcagtgcacgatcggcgcgggcaaagcgctcacggaaggcttgcaccagttggcgcaggcgacgggaacgactttgtacatggttttgctcgccgcgtacaacgtgctgctcgccaagtatgccgggcaggaggacatcatcgtcggcacgccgattacaggcagatcccatgccgatctcgaaccgatcgtcggcatgttcgtgaacaccttggcgatgcgaaacaaaccgcagcgcgaaaagacttttagcgagtttttgcaagaagtcaagcaaaatgcgctggatgcgtacggccatcaggattacccgtttgaagaactggtggaaaagctcgcgatcgcgcgcgatttgagccgaaatccgctgtttgacaccgtgtttacgttccaaaacagcacggaagaggtcatgacgctgcctgaatgcacgcttgcgccgtttatgacggacgaaacaggccagcacgccaagttcgacttgactttcagcgctacggaagagcgggaagaaatgacgattggcgtggagtacagcacaagcttgtttacgcgggaaacgatggaacggttcagccgccacttcctgacgattgcagcgagcatcgtgcaaaatccgcacatccgtctgggcgagatcgacatgcttttgccagaagaaaaacagcagattttggccgggttcaacgatacggcagtcagctatgcgctggacaaaacgctgcaccagctattcgaagagcaggtcgacaaaacaccggatcaggcagcgcttctctttagcgagcaatcgctgacgtacagcgaactgaacgagcgagcaaacagactggcaagggtcctgcgcgcaaaaggagtcggaccggaccgtctggtagcgatcatggcggagcgctcgccggaaatggtgatcggtattctcggtattttgaaggcaggcggcgcttatgttcccgtcgatcccggctatccgcaggagcgcattcagtacctgctcgaagatagcaacgcagccctgctgctcagccaggcgcatctgttgccgctgttggcccaggtgtcaagcgagctgccggagtgccttgatctgaacgctgaactggatgccggactgagcggctccaacctgccagctgtcaaccaaccgactgaccttgcctacgtcatctatacatccggtacgaccggcaagccgaagggtgtcatgatcccgcatcaaggaatcgtgaactgcttgcagtggagaagagacgaatacgggttcgggccgagtgacaaggcgttgcaagtgttctcctttgccttcgacggttttgtagccagcttgttcgctccgctgctcggaggggcaacgtgcgtgttgccgcaagaagcagctgccaaagacccggtcgcgctgaaaaaactgatggccgcaacggaagtcacccattactacggcgtaccgagtctgttccaggccattctcgattgctcgacgacaaccgacttcaatcagttgcgttgcgtcactttgggcggcgagaagctgcctgtgcagcttgtgcaaaaaacaaaagaaaagcatccggcaatcgagatcaacaacgagtacggcccgacggaaaacagcgtcgtcaccaccatctcgcgctcgattgaagcggggcaagcgatcacgattggccgaccgcttgcgaacgtccaagtctacattgtagatgagcagcatcacttgcagccgattggcgtggtcggtgagctgtgcatcggcggagccgggcttgccagaggctatctgaacaaaccggagctgaccgcagagaagtttgtcgcaaatccgttccgaccaggcgagcgcatgtacaaaacaggcgacttggtaaaatggcggacggatggcacgatcgagtacatcggccgcgcagacgaacaggtcaaggtgagagggtatcgcatcgagatcggcgagatcgagagcgccgtactcgcttaccagggcatcgatcaagcggtggtcgttgcgcgagacgatgacgctacggctggttcctatctttgcgcctactttgtcgcagcaacagccgtgtccgtatccggcttgagaagccatctggccaaagagctgcctgcttacatgattccgagctatttcgtcgagctggatcagctgccgctttccgccaatggaaaagtggatcgcaaagctttgccgaagccgcaacagtccgatgcgaccacgcgcgaatacgtggccccgaggaatgcgaccgaacagcaactggcagccatctggcaagaagttttgggagtagagccaatcggcatcaccgaccagttctttgaactcggaggacattccttaaaagctacgctgttgattgccaaagtgtatgagtacatgcaaatcgagctgccgctgaatctcatcttccagtatccgacgatcgaaaaggtggccgatttcatcacgcataagcgctttgagagcagatacggcacagccattttgttaaatcaggagacggcgcgaaacgtattttgcttcacgccgatcggcgcacaaagcgtgtactaccagaagcttgcggcggaaattcaaggcgtctctttgtacagctttgatttcatccaggatgacaaccggatggagcagtatatcgcggcgatcaccgcaattgatccaagcggtccgtacacgctcatgggctactcctcgggaggcaatctggcttttgaagtggcgaaagaactggaggagcggggctatggcgtcaccgacatcatcttgttcgactcgtactggaaagacaaggcgattgagcggactgtcgcggaaacagaaaacgacattgcccagctattcgccgagattggcgaaaacaccgagatgttcaacatgacgcaagaagacttccagctgtacgccgccaatgagtttgtcaagcaaagcttcgttcgcaaaacggtcagctatgtgatgttccataacaatctggtcaataccggaatgaccactgccgcgatccacctcatccaatccgagctggaagcagacgaggaagctccggtggcagccaagtggaacgaatcagcctgggcaaacgcaacgcaacgactgctgacgtacagcgggcacggaatccactcgcgcatgctggcgggcgattacgcgtcgcaaaatgcttcgattttgcaaaacatcctgcaagaactgttcatcctgaaataa

SEQ ID NO: 23 - DNA, 6507 bp, Artificial Sequence
  NRPS being a synthetase of a fusion peptide consisting of Valine and Indigoidine. Due to its sterical advantages, Valine may be used as a spacer for other tags.
atgtatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagagttcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccacgagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 24 - DNA, 9609 bp, Artificial Sequence
  NRPS synthesizing a Indigoidine-tagged Dipeptide consisting of two Valine-monomers.
atgtatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatattgccacgagcggaaaagagacgtatgtgccgatcgagcctgcaccgttgcaagagtattatcctgtttcatctgcgcaaaagcggatgtatgtcctgcgccagtttgcggacacaggcacggtttataacatgccgagcgcgttgtatatcgaaggcgatctggatcggaagcgttttgaagccgccatccacggattggtcgagcggcacgaatcgctgcgcacatccttccacaccgtaaatggcgagcctgtccagcgcgtacacgagcatgtcgagctgaatgtgcagtacgcggaagtgacggaagcgcaagtggagccaaccgtcgagtcgttcgtgcaagcatttgatctgacaaaagctccgctattgcgggtcggacttttcaagctggcagcgaaacggcatctgttcctgctggatatgcatcacatcatctcggatggcgtctcggccggaatcattatggaagagttctcgaagctgtatcgaggcgaagaactgcctgcgctttccgtccattacaaagatttcgccgtctggcagtctgaactgttccagagcgacgtctataccgagcatgaaaactactggctgaacgcgttttctggcgacattccggtgcttaacttgccagccgatttttctcgtccgctgacacagagctttgaaggagattgcgtttcgttccaggcagacaaagcgttgctggacgatcttcacaagctcgctcaggagagccaatcgacgttgttcatggtattgctggcggcttacaatgtgctgcttgccaagtacagcggacaggaagacatcgtcgtcggcacaccgattgcgggcagatcgcacgccgatatcgagaacgttctggggatgtttgtcaacacgctcgctttgcgcaactatccggtcgagacgaaacacttccaggcatttttggaagaggtcaagcaaaatacgctgcaagcatacgcccatcaagattatccgttcgaagcactggtcgaaaagctggacatccagcgggatctcagccgcaatccgctgtttgacaccatgtttattttgcaaaacctggaccaaaaagcttacgagctggatgggctgaaactggaggcatatccggcacaagcaggcaacgccaaattcgatctcacgctggaagcgcacgaggacgagacaggcattcattttgcgctcgtctactcgaccaaattgttccagcgagaatcaatcgaaagaatggcgggtcacttcctgcaagtgctgcgccaagtcgttgccgaccaagcaactgccttgcgcgagatcagcctgctcagcgaggaagagcgccgaattgtgaccgttgatttcaacaacacgtttgcctatccgcgcgatctgacgattcaggagctgttcgagcagcaggcagcaaaaactccggagcatgcagcggtcgtgatggacggacagatgctgacgtatcgggagctgaacgaaaaagcgaaccagctcgcccatgtccttcgtcaaaacggagtcgggaaagagagcatcgtcggtctgctcgcagatcgttcgctggaaatgattacaggcatcatggggattctcaaagcgggcggcgcctacctgggactggacccggagcatccgtccgaacgcctggcttacatgttggaagatggcggcgtgaaagttgtcctcgtgcaaaagcacttgctgccgctcgtcggcgaagggctgatgccaatcgttttggaagaggagagcctgcgcccggaagattgcggcaatccggcgattgtcaacggtgcgagtgacctggcttatgtgatgtacacctcaggctctacaggcaagccaaaaggagtcatggtcgagcatcgcaacgtcacccgcttggtcatgcatacgaattacgtgcaagtgcgcgagagcgaccggatgattcaaaccggcgcgattggcttcgacgccatgacatttgagatttttggagccttgctgcacggggccagcctgtatttggtgagcaaggacgtcttgctggatgccgaaaagctgggcgacttcctgcggacgaatcagattacgaccatgtggctgacctcgccgctcttcaaccagctttcgcaagacaatccggcgatgtttgacagcttgcgcgccttgatcgtcggtggcgaagcgttgtcgccgaagcacatcaaccgggtaaaaagtgcccttcctgacctggaaatctggaacggatacggcccgaccgaaaacacgaccttctcgacgtgctatttgattgagcagcattttgaagagcagattccgatcggcaagccgattgcaaactccaccgcgtatatcgtcgacggcaacaatcagccgcagccgatcggcgtaccgggtgaactgtgcgtcggtggtgacggtgtcgcaagaggctatgtgaacaagccggaattaaccgccgaaaagtttgtgcccaatccgtttgcgcctggcgaaacgatgtatcgcaccggagatttggcgagatggctgccggatgggacgattgagtatttgggccgaatcgaccagcaggtcaaaatcaggggataccggatcgagcttggggaaatcgagacggtcttgtcccagcaggcacaagtaaaagaagcagtcgtggccgtgatcgaggaggcgaacgggcaaaaagctctctgcgcttactttgtgccagaacaggccgtcgacgccgcagagctgcgagaagcgatgtccaaacaattgcctggctacatggtccctgcttactatgtgcaaatggaaaagctgccgttgaccgcgaacggaaaggtcgaccgccgggcattgccgcagccatccggcgagcggacgacaggaagcgcctttgtcgctgcgcaaaatgataccgaagcgaagctgcaacagatttggcaagaagttttgggcattccggcaatcggcattcacgacaacttctttgaaatcggcggtcattccttgaaggcgatgaacgtcatcacgcaagtccataaaacattccaggtggagctgccgttaaaagcgctgtttgccactccgacgatccatgagttggctgcgcatatttcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccactagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 25 - protein, 1284 aa, Photorhabdus luminescens
MLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 26 - DNA, 4776 bp, Artificial Sequence
  minimal construct C(of TycC2)-Ind
tcggaaaaaaccgagtacaccgcgattcaacccgtggcagcgcaggagttttacccggtttcatctgcgcaaaaaagaatgtatatcctgcaacagttcgaaggcaacggaatcagctacaacatttcgggtgcgattctcctggaaggaaagctggactacgcccggtttgccagcgctgtgcaacagctggcagagcgccacgaagctttgcgcacctcgttccaccggatcgacggcgagcctgtgcaaaaagtgcacgaggaagtagaagtgccgcttttcatgctggaggctcccgaagaccaggcggagaaaatcatgcgcgagtttgtccgtccgtttgatctcggggtcgctccgctgatgcgaacaggtttgctcaagctgggcaaagaccgccatttgtttttgctcgacatgcaccatatcatctcggacggcgtttcttcgcaaattttgctgcgtgaatttgccgagttgtaccagggagcagacttgcagccgctttcgctgcaatacaaagatttcgctgcttggcaaaatgagctgtttcagacggaggcatacaagaagcaggagcagcactggctgaacacgtttgctgatgaaattccgctcttgaacctgccgactgactatccgcgccctagcgtgcaaagctttgcaggcgatctcgtcctttttgccgccggaaaagaactgctggagcggttgcaacaggtagcgtcagaaacaggcaccaccttgtacatgattttgcttgccgcctacaatgtgctgctgtccaagtataccggccaggaagacatcatcgtcgggacgcctgtcgctggacgttcccatgcggacgtggaaaacatcatgggcatattcgtgaacacattggcgctgcgcaaccagcctgccagcagcaaaacgatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagagttcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctaaacccacgagtctggcttatattatttatacctctggtagcacgggtaagccaaagggagtgatgattgaacatcacagtattgtaaatcaaatgagatttcttgcaaaagcgttcaaattaggatgtcattcccggattttacagaaaacaccaatgagttttgatgcggctcaatgggaaattctagcgcctgcaattggtggtcaagtgattatgggtcctttaggttgctatcgcgatccggatgcaattattaaaaccattcttcagcatcaagtaacgactttgcaatgtgttcctactttgctacaagcgttactggataatcctaattttttggattgcttatcattgactcaagtattcagtgggggagaagcgctgacaaccaaattagccacgcaatttttgaatagttttactcactgtgaattaatcaatttatatggcccgacagaatgtacgattaattcatcatttttccgggtgacaaatgagactttgccgaattatcaaacctctatttcgattggtgcacctgtagataataccgaatactacgttcttgatgatgatagattacctgtggcggttggcgaaattggcgagctttatatttcgggtgctcaattagcacgtggttatttgcataaaccagaaatgacaaaagataaatttatttgtaatcaccttgtatcaggaactcaacatcaatggttatatcgaacgggagatctggtaaccagaggggctgatggtaatacttattttgttggtcgggttgatagccaggtcaaattacgaggttaccgtattgagcttgatgaaatacgccatgcgattgaagaacatagctggataaagacggcggcaatgttaattaagaaggatgccagaacgggtttccaaaatctcatcgcgtgtgtggaattagatgagaaagaagctgcattgatggatcaaggtaatagtagctcacatcacaaatcaaaagccgataaactacaggtgaaagcccaactttctaattctggttgtcgaagtgaagagttatgtgaaaatcgccctacattcttacttccttatcaagaaggggagataaaacagagagaatatgcatttggacgcaagacatatcgctattttgagggaacagaaataacggtagagaaattaaaaaaattgctgacagccactcaatcgaatgaaattagctctttgccactgagtcatctaaccctgaatgatttcggttatgcattgcgttattttggtcagtttaccagccatcaacgtttattgcccaaatatgcctatgcttcaccgggtgctctctatgcgacacaaatgtattttgaattgcataatgttctcggtttggatgcggggatttactattatcatccagtgacacataagttaataaaaatttcaacattgagtcgtcggcaaatgccaacgataaaagtgcattttattggcaagcatgaagccattgagcccgtttataagaacaatatacaagaagttctggaaatggaagcgggccatatgatgggtctttttgatgacgtattaccggaaattggcttgagtattggtaaaagtgaatatcaagatgaatgtccagattggtatgatggtgatattcaggattattatcttggtgcatttgaaatatgtagctatgaacatggattgccgccatttgagactgatatttatttacaaacacatgcccataaaatacctgagatgccgtgtggtttatatcacttttctaacggggaatttgtacgaataagtgatgatattgtccgaaaaaaggatgttattgcgattaatcagcaagtttatgatcgctccagttttggcgtgtcaattattccacgctgtgtccctgaatggcattattatataacactgggtcgtcggttacatgcgttacaaagtaatccattgtatattggattaatgtcatctggttacagttcgaagagcaataacgatttaccttcggcgaaaaggatgcgatctattctcaatgcacttgatagacctatggcggcattttatttctgcataggtgggggtattagccaagcgcaatatatgtgtgaaggcatgaaagaagatgttgttcatatgaaagggccagttgaaatcattaaagatgatcttcaacaacaactccctcaatatatgattccaaataaggtattagttttcgataaattacctttgacggccaatggaaaagtggattatcaatctttatcagaatctaaagccgtggagaatgtttcaacacagcgtctattggtgccattacatacagatactgaaataaggcttggaaaaatttggatggaagtactgaaatgggattcagtatctgccctcgatgattttttcgaaagtgggggtaattctttgatggccgttgcaatggttaataagatcaatgcggcctttaatattcgttttccgttacagatactttttcaatctcctaatatagcagaattggctaagtggattgaacagacagactctaaaacaatatcaagattaattttattgaatcaggcaagcaaagaccccatttactgttggccgggtttgggcggatatcctatgagtttgagattgcttgctaataaagtcgttcctgatcgggcattttatggaatacaggcatatgggataaacgagagtgaaataccgttttcttctatccagagaatggcagaagaggatattaaagagataaagaaaatacagccagaagggccatatatattgtggggatattcatttggtgcccgagtagcatttgaagttgcataccagcttgaacaagcgggagaagaagttaacgcattgaatttattggctccgggatctcctcatcttgatatgaagcaagcggaatatatggataaaggcgctgaatttactaatccggcttttgttaaaatacttttttctgtattttctcgttcaatcaacagcccaatggttaaaacttgcttagaacaagtaaatagtgaaacgacatttattaactttatatgtagtcgttttaaaaacttggaaccatcattagtaaaacgtatcgttaggattgtgactttgacttatgatttcaagtacagtattgatgagctttatcacagacacctaaaggcacctataactattttcaaggcgaatagagataatgattcatttatcgaggaatcggatgtgatttcatcaatgtcgcctaaaataattgaattaatatcggatcactatcaactgttggaaagtgaaggtgttgctgagattgagaaaataatctaa

SEQ ID NO: 27 - protein, 2327 aa, Artificial Sequence
  NRPSase of a fusion peptide consisting of Asparagine and Indigoidine
MQTNKQQTFSELLQTVQKQALASATYDFAPLYEIQSTTVLKQELIDHLVTFENYPDHSMKHLEESLGFQFTVESGDEQTSYDLNVVVALAPSNELYVKLSYNAAVYESSFVNRIEGHLRTVIDQVIGNPHVHLHEIGIITEEEKQQLLVAYNDTAAEYPRDKTIFELIAEQASRTPAKAAVVCGEDTLTYQELMERSAQLANALREKGIASGSIVSIMAEHSLELIVAIMAVLRSGAAYLPIDPEYPQDRIQYLLDDSQTTLLLTQSHLQPNIRFAGSVLYLDDRSLYEGGSTSFAPESKPDDLAYMIYTSGSTGNPKGAMITHQGLVNYIWWANKVYVQGEAVDFPLYSSISFDLTVTSIFTPLLSGNTIHVYRGADKVQVILDIIKDNKVGIIKLTPTHLKLIEHIDGKASSIRRFIVGGENLPTKLAKQIYDHFGENVQIFNEYGPTETVVGCMIYLYDPQTTTQESVPIGVPADNVQLYLLDASMQPVPVGSLGEMYIAGDGVAKGYFNRPELTKEKFIDNPFRPGTKMYRTGDLAKWLPDGNMEYAGRMDYQVKIRGHRIEMGEIETRLTQHEAVKEAVVIVEKDESGQNVLYAYLVSERELTVAELREFLGRTLPSYMIPSFFIRLAEIPLTANGKVERKKLPKPAGAVVTGTAYAAPQNEIEAKLAEIWQQVLGISQVGIHDDFFDLGGHSLKAMTVVFQVSKALEVELPVKALFEHPTVAELARFLSRSEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 28 - protein, 3221 aa, Artificial Sequence
  NRPSase synthesizing a Indigoidine-tagged Dipeptide consisting of Ornithine and Valine
MLHSFLATKTAYPTDKTFQKLFEEQVEKTPNEIAVLFGNEQLTYQELNAKANQLARVLRRKGVKPESTVGILVDRSLYMVIGMLAVLKAGGTFVPIDPDYPLERQAFMLEDSEAKLLLTLQKMNSQVAFPYETFYLDTETVDQEETGNLEHVAQPENVAYIIYTSGTTGKPKGVVIEHRSYANVAFAWKDEYHLDSFPVRLLQMASFAFDVSTGDFARALLTGGQLVICPNGVKMDPASLYETIRRHEITIFEATPALIMPLMHYVYENELDMSQMKLLILGADSCPAEDFKTLLARFGQKMRIINSYGVTEACIDTSYYEETDVTAIRSGTVPIGKPLPNMTMYVVDAHLNLQPVGVVGELCIGGAGVARGYLNRPELTEEKFVPNPFAPGERLYRTGDLAKWRADGNVEFLGRNDHQVKIRGVRIELGEIETQLRKLDGITEAVVVAREDRGQEKELCAYVVADHKLDTAELRANLLKELPQAMIPAYFVTLDALPLTANGKVDRRSLPAPDVTMLRTTEYVAPRSVWEARLAQVWEQVLNVPQVGALDDFFALGGHSLRAMRVLSSMHNEYQVDIPLRILFEKPTIQELAAFIETSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 29 - protein, 4256 aa, Artificial Sequence
  NRPSase synthesizing a Indigoidine-tagged Tripeptide consisting of Ornithine and two Valines
MLHSFLATKTAYPTDKTFQKLFEEQVEKTPNEIAVLFGNEQLTYQELNAKANQLARVLRRKGVKPESTVGILVDRSLYMVIGMLAVLKAGGTFVPIDPDYPLERQAFMLEDSEAKLLLTLQKMNSQVAFPYETFYLDTETVDQEETGNLEHVAQPENVAYIIYTSGTTGKPKGVVIEHRSYANVAFAWKDEYHLDSFPVRLLQMASFAFDVSTGDFARALLTGGQLVICPNGVKMDPASLYETIRRHEITIFEATPALIMPLMHYVYENELDMSQMKLLILGADSCPAEDFKTLLARFGQKMRIINSYGVTEACIDTSYYEETDVTAIRSGTVPIGKPLPNMTMYVVDAHLNLQPVGVVGELCIGGAGVARGYLNRPELTEEKFVPNPFAPGERLYRTGDLAKWRADGNVEFLGRNDHQVKIRGVRIELGEIETQLRKLDGITEAVVVAREDRGQEKELCAYVVADHKLDTAELRANLLKELPQAMIPAYFVTLDALPLTANGKVDRRSLPAPDVTMLRTTEYVAPRSVWEARLAQVWEQVLNVPQVGALDDFFALGGHSLRAMRVLSSMHNEYQVDIPLRILFEKPTIQELAAFIETSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHIATSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 30 - protein, 2194 aa, Artificial Sequence
  NRPSase of a fusion peptide consisting of Phenylalanine and Indigoidine
MLANQANLIDNKRELEQHALVPYAQGKSIHQLFEEQAEAFPDRVAIVFENRRLSYQELNRKANQLARALLEKGVQTDSIVGVMMEKSIENVIAILAVLKAGGAYVPIDIEYPRDRIQYILQDSQTKIVLTQKSVSQLVHDVGYSGEVVVLDEEQLDARETANLHQPSKPTDLAYVIYTSGTTGKPKGTMLEHKGIANLQSFFQNSFGVTEQDRIGLFASMSFDASVWEMFMALLSGASLYILSKQTIHDFAAFEHYLSENELTIITLPPTYLTHLTPERITSLRIMITAGSASSAPLVNKWKDKLRYINAYGPTETSICATIWEAPSNQLSVQSVPIGKPIQNTHIYIVNEDLQLLPTGSEGELCIGGVGLARGYWNRPDLTAEKFVDNPFVPGEKMYRTGDLAKWLTDGTIEFLGRIDHQVKIRGHRIELGEIESVLLAHEHITEAVVIAREDQHAGQYLCAYYISQQEATPAQLRDYAAQKLPAYMLPSYFVKLDKMPLTPNDKIDRKALPEPDLTANQSQAAYHPPRTETESILVSIWQNVLGIEKIGIRDNFYSLGGDSIQAIQVVARLHSYQLKLETKDLLNYPTIEQVAELARFLSRSEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 31 - protein, 4744 aa, Artificial Sequence
  NRPSase synthesizing a Indigoidine-tagged Tripeptide consisting of Phenylalanine, Ornithine and Leucine
MLANQANLIDNKRELEQHALVPYAQGKSIHQLFEEQAEAFPDRVAIVFENRRLSYQELNRKANQLARALLEKGVQTDSIVGVMMEKSIENVIAILAVLKAGGAYVPIDIEYPRDRIQYILQDSQTKIVLTQKSVSQLVHDVGYSGEVVVLDEEQLDARETANLHQPSKPTDLAYVIYTSGTTGKPKGTMLEHKGIANLQSFFQNSFGVTEQDRIGLFASMSFDASVWEMFMALLSGASLYILSKQTIHDFAAFEHYLSENELTIITLPPTYLTHLTPERITSLRIMITAGSASSAPLVNKWKDKLRYINAYGPTETSICATIWEAPSNQLSVQSVPIGKPIQNTHIYIVNEDLQLLPTGSEGELCIGGVGLARGYWNRPDLTAEKFVDNPFVPGEKMYRTGDLAKWLTDGTIEFLGRIDHQVKIRGHRIELGEIESVLLAHEHITEAVVIAREDQHAGQYLCAYYISQQEATPAQLRDYAAQKLPAYMLPSYFVKLDKMPLTPNDKIDRKALPEPDLTANQSQAAYHPPRTETESILVSIWQNVLGIEKIGIRDNFYSLGGDSIQAIQVVARLHSYQLKLETKDLLNYPTIEQVALFVKSTTRKSDQGIIAGNVPLTPIQKWFFGKNFTNTGHWNQSSVLYRPEGFDPKVIQSVMDKIIEHHDALRMVYQHENGNVVQHNRGLGGQLYDFFSYNLTAQPDVQQAIEAETQRLHSSMNLQEGPLVKVALFQTLHGDHLFLAIHHLVVDGISWRILFEDLATGYAQALAGQAISLPEKTDSFQSWSQWLQEYANEADLLSEIPYWESLESQAKNVSLPKDYEVTDCKQKSVRNMRIRLHPEETEQLLKHANQAYQTEINDLLLAALGLAFAEWSKLAQIVIHLEGHGREDIIEQANVARTVGWFTSQYPVLLDLKQTAPLSDYIKLTKENMRKIPRKGIGYDILKHVTLPENRGSLSFRVQPEVTFNYLGQFDADMRTELFTRSPYSGGNTLGADGKNNLSPESEVYTALNITGLIEGGELVLTFSYSSEQYREESIQQLSQSYQKHLLAIIAHCTEKKEVERTAHIAESAFEQFETIQPVEPAAFYPVSFAQKRMYILHQFEGSGISYNVPSVLVLEGKLDYDRFAAAIQSLVKRHESLRTSFHSVNGEPLQRVHPDVELPVRLLEATEDQSESLIQELIQPFDLEIAPLFRVNLIKLGAERHLFFMDMHHIISDGVSLAVIVEEIASLYAGKQLSDLRIQYKDFAVWQTKLAQSDRFQKQEDFWTRTFAGEIPLLNLPHDYPRPSVQSFDGDTVALGTGHHLLEQLRKLAAETGTTLFMVLLAAYHVLLSKYAGQEEIVVGTPIAGRSHADVERIVGMFVNTLALKNTAAGSLSFRAFLEDVKQNALHAFEHQDYPFEHLVEKLQVRRDLSRNPLFDTMFSLGLAESAEGEVADLKVSPYPVNGHIAKFDLSLDAMEKQDGLLVQFSYCTKLFAKETVDRLAAHYVQLLQTITADPDIELARISVLSKAETEHMLHSFLATKTAYPTDKTFQKLFEEQVEKTPNEIAVLFGNEQLTYQELNAKANQLARVLRRKGVKPESTVGILVDRSLYMVIGMLAVLKAGGTFVPIDPDYPLERQAFMLEDSEAKLLLTLQKMNSQVAFPYETFYLDTETVDQEETGNLEHVAQPENVAYIIYTSGTTGKPKGVVIEHRSYANVAFAWKDEYHLDSFPVRLLQMASFAFDVSTGDFARALLTGGQLVICPNGVKMDPASLYETIRRHEITIFEATPALIMPLMHYVYENELDMSQMKLLILGADSCPAEDFKTLLARFGQKMRIINSYGVTEACIDTSYYEETDVTAIRSGTVPIGKPLPNMTMYVVDAHLNLQPVGVVGELCIGGAGVARGYLNRPELTEEKFVPNPFAPGERLYRTGDLAKWRADGNVEFLGRNDHQVKIRGVRIELGEIETQLRKLDGITEAVVVAREDRGQEKELCAYVVADHKLDTAELRANLLKELPQAMIPAYFVTLDALPLTANGKVDRRSLPAPDVTMLRTTEYVAPRSVWEARLAQVWEQVLNVPQVGALDDFFALGGHSLRAMRVLSSMHNEYQVDIPLRILFEKPTIQELAAFIEETAKGNVFSIEPVQKQAYYPVSSAQKRMYILDQFEGVGISYNMPSTMLIEGKLERTRVEAAFQRLIARHESLRTSFAVVNGEPVQNIHEDVPFALAYSEVTEQEARELVSSLVQPFDLEVAPLIRVSLLKIGEDRYVLFTDMHHSISDGVSSGILLAEWVQLYQGDVLPELRIQYKDFAVWQQEFSQSAAFHKQEAYWLQTFADDIPVLNLPTDFTRPSTQSFAGDQCTIGAGKALTEGLHQLAQATGTTLYMVLLAAYNVLLAKYAGQEDIIVGTPITGRSHADLEPIVGMFVNTLAMRNKPQREKTFSEFLQEVKQNALDAYGHQDYPFEELVEKLAIARDLSRNPLFDTVFTFQNSTEEVMTLPECTLAPFMTDETGQHAKFDLTFSATEEREEMTIGVEYSTSLFTRETMERFSRHFLTIAASIVQNPHIRLGEIDMLLPEEKQQILAGFNDTAVSYALDKTLHQLFEEQVDKTPDQAALLFSEQSLTYSELNERANRLARVLRAKGVGPDRLVAIMAERSPEMVIGILGILKAGGAYVPVDPGYPQERIQYLLEDSNAALLLSQAHLLPLLAQVSSELPECLDLNAELDAGLSGSNLPAVNQPTDLAYVIYTSGTTGKPKGVMIPHQGIVNCLQWRRDEYGFGPSDKALQVFSFAFDGFVASLFAPLLGGATCVLPQEAAAKDPVALKKLMAATEVTHYYGVPSLFQAILDCSTTTDFNQLRCVTLGGEKLPVQLVQKTKEKHPAIEINNEYGPTENSVVTTISRSIEAGQAITIGRPLANVQVYIVDEQHHLQPIGVVGELCIGGAGLARGYLNKPELTAEKFVANPFRPGERMYKTGDLVKWRTDGTIEYIGRADEQVKVRGYRIEIGEIESAVLAYQGIDQAVVVARDDDATAGSYLCAYFVAATAVSVSGLRSHLAKELPAYMIPSYFVELDQLPLSANGKVDRKALPKPQQSDATTREYVAPRNATEQQLAAIWQEVLGVEPIGITDQFFELGGHSLKATLLIAKVYEYMQIELPLNLIFQYPTIEKVADFITSEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 32 - protein, 5777 aa, Artificial Sequence
  NRPSase synthesizing a Valine-Indigoidine- tagged Tripeptide consisting of Phenylalanine, Ornithine and Leucine. Valine is here used as spacer.
MLANQANLIDNKRELEQHALVPYAQGKSIHQLFEEQAEAFPDRVAIVFENRRLSYQELNRKANQLARALLEKGVQTDSIVGVMMEKSIENVIAILAVLKAGGAYVPIDIEYPRDRIQYILQDSQTKIVLTQKSVSQLVHDVGYSGEVVVLDEEQLDARETANLHQPSKPTDLAYVIYTSGTTGKPKGTMLEHKGIANLQSFFQNSFGVTEQDRIGLFASMSFDASVWEMFMALLSGASLYILSKQTIHDFAAFEHYLSENELTIITLPPTYLTHLTPERITSLRIMITAGSASSAPLVNKWKDKLRYINAYGPTETSICATIWEAPSNQLSVQSVPIGKPIQNTHIYIVNEDLQLLPTGSEGELCIGGVGLARGYWNRPDLTAEKFVDNPFVPGEKMYRTGDLAKWLTDGTIEFLGRIDHQVKIRGHRIELGEIESVLLAHEHITEAVVIAREDQHAGQYLCAYYISQQEATPAQLRDYAAQKLPAYMLPSYFVKLDKMPLTPNDKIDRKALPEPDLTANQSQAAYHPPRTETESILVSIWQNVLGIEKIGIRDNFYSLGGDSIQAIQVVARLHSYQLKLETKDLLNYPTIEQVALFVKSTTRKSDQGIIAGNVPLTPIQKWFFGKNFTNTGHWNQSSVLYRPEGFDPKVIQSVMDKIIEHHDALRMVYQHENGNVVQHNRGLGGQLYDFFSYNLTAQPDVQQAIEAETQRLHSSMNLQEGPLVKVALFQTLHGDHLFLAIHHLVVDGISWRILFEDLATGYAQALAGQAISLPEKTDSFQSWSQWLQEYANEADLLSEIPYWESLESQAKNVSLPKDYEVTDCKQKSVRNMRIRLHPEETEQLLKHANQAYQTEINDLLLAALGLAFAEWSKLAQIVIHLEGHGREDIIEQANVARTVGWFTSQYPVLLDLKQTAPLSDYIKLTKENMRKIPRKGIGYDILKHVTLPENRGSLSFRVQPEVTFNYLGQFDADMRTELFTRSPYSGGNTLGADGKNNLSPESEVYTALNITGLIEGGELVLTFSYSSEQYREESIQQLSQSYQKHLLAIIAHCTEKKEVERTAHIAESAFEQFETIQPVEPAAFYPVSFAQKRMYILHQFEGSGISYNVPSVLVLEGKLDYDRFAAAIQSLVKRHESLRTSFHSVNGEPLQRVHPDVELPVRLLEATEDQSESLIQELIQPFDLEIAPLFRVNLIKLGAERHLFFMDMHHIISDGVSLAVIVEEIASLYAGKQLSDLRIQYKDFAVWQTKLAQSDRFQKQEDFWTRTFAGEIPLLNLPHDYPRPSVQSFDGDTVALGTGHHLLEQLRKLAAETGTTLFMVLLAAYHVLLSKYAGQEEIVVGTPIAGRSHADVERIVGMFVNTLALKNTAAGSLSFRAFLEDVKQNALHAFEHQDYPFEHLVEKLQVRRDLSRNPLFDTMFSLGLAESAEGEVADLKVSPYPVNGHIAKFDLSLDAMEKQDGLLVQFSYCTKLFAKETVDRLAAHYVQLLQTITADPDIELARISVLSKAETEHMLHSFLATKTAYPTDKTFQKLFEEQVEKTPNEIAVLFGNEQLTYQELNAKANQLARVLRRKGVKPESTVGILVDRSLYMVIGMLAVLKAGGTFVPIDPDYPLERQAFMLEDSEAKLLLTLQKMNSQVAFPYETFYLDTETVDQEETGNLEHVAQPENVAYIIYTSGTTGKPKGVVIEHRSYANVAFAWKDEYHLDSFPVRLLQMASFAFDVSTGDFARALLTGGQLVICPNGVKMDPASLYETIRRHEITIFEATPALIMPLMHYVYENELDMSQMKLLILGADSCPAEDFKTLLARFGQKMRIINSYGVTEACIDTSYYEETDVTAIRSGTVPIGKPLPNMTMYVVDAHLNLQPVGVVGELCIGGAGVARGYLNRPELTEEKFVPNPFAPGERLYRTGDLAKWRADGNVEFLGRNDHQVKIRGVRIELGEIETQLRKLDGITEAVVVAREDRGQEKELCAYVVADHKLDTAELRANLLKELPQAMIPAYFVTLDALPLTANGKVDRRSLPAPDVTMLRTTEYVAPRSVWEARLAQVWEQVLNVPQVGALDDFFALGGHSLRAMRVLSSMHNEYQVDIPLRILFEKPTIQELAAFIEETAKGNVFSIEPVQKQAYYPVSSAQKRMYILDQFEGVGISYNMPSTMLIEGKLERTRVEAAFQRLIARHESLRTSFAVVNGEPVQNIHEDVPFALAYSEVTEQEARELVSSLVQPFDLEVAPLIRVSLLKIGEDRYVLFTDMHHSISDGVSSGILLAEWVQLYQGDVLPELRIQYKDFAVWQQEFSQSAAFHKQEAYWLQTFADDIPVLNLPTDFTRPSTQSFAGDQCTIGAGKALTEGLHQLAQATGTTLYMVLLAAYNVLLAKYAGQEDIIVGTPITGRSHADLEPIVGMFVNTLAMRNKPQREKTFSEFLQEVKQNALDAYGHQDYPFEELVEKLAIARDLSRNPLFDTVFTFQNSTEEVMTLPECTLAPFMTDETGQHAKFDLTFSATEEREEMTIGVEYSTSLFTRETMERFSRHFLTIAASIVQNPHIRLGEIDMLLPEEKQQILAGFNDTAVSYALDKTLHQLFEEQVDKTPDQAALLFSEQSLTYSELNERANRLARVLRAKGVGPDRLVAIMAERSPEMVIGILGILKAGGAYVPVDPGYPQERIQYLLEDSNAALLLSQAHLLPLLAQVSSELPECLDLNAELDAGLSGSNLPAVNQPTDLAYVIYTSGTTGKPKGVMIPHQGIVNCLQWRRDEYGFGPSDKALQVFSFAFDGFVASLFAPLLGGATCVLPQEAAAKDPVALKKLMAATEVTHYYGVPSLFQAILDCSTTTDFNQLRCVTLGGEKLPVQLVQKTKEKHPAIEINNEYGPTENSVVTTISRSIEAGQAITIGRPLANVQVYIVDEQHHLQPIGVVGELCIGGAGLARGYLNKPELTAEKFVANPFRPGERMYKTGDLVKWRTDGTIEYIGRADEQVKVRGYRIEIGEIESAVLAYQGIDQAVVVARDDDATAGSYLCAYFVAATAVSVSGLRSHLAKELPAYMIPSYFVELDQLPLSANGKVDRKALPKPQQSDATTREYVAPRNATEQQLAAIWQEVLGVEPIGITDQFFELGGHSLKATLLIAKVYEYMQIELPLNLIFQYPTIEKVADFITTSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 33 - protein, 3251 aa, Artificial Sequence
  NRPSase synthesizing a Indigoidine-tagged Dipeptide consisting of Proline and Leucine
MDCVANNSGVELCQIPLLTEAETSQLLAKRTETAADYPAATMHELFSRQAEKTPEQVAVVFADQHLTYRELDEKSNQLARFLRKKGIGTGSLVGTLLDRSLDMIVGILGVLKAGGAFVPIDPELPAERIAYMLTHSRVPLVVTQNHLRAKVTTPTETIDINTAVIGEESRAPIESLNQPHDLFYIIYTSGTTGQPKGVMLEHRNMANLMHFTFDQTNIAFHEKVLQYTTCSFDVCYQEIFSTLLSGGQLYLITNELRRHVEKLFAFIQEKQISILSLPVSFLKFIFNEQDYAQSFPRCVKHIITAGEQLVVTHELQKYLRQHRVFLHNHYGPSETHVVTTCTMDPGQAIPELPPIGKPISNTGIYILDEGLQLKPEGIVGELYISGANVGRGYLHQPELTAEKFLDNPYQPGERMYRTGDLALWLPDGQLEFLGRIDHQVKIRGHRIELGEIESRLLNHPAIKEAVVIDRADETGGKFLCAYVVLQKALSDEEMRAYLAQALPEYMIPSFFVTLERIPVTPNGKTDRRALPKPEGSAKTKADYVAPTTELEQKLVAIWEQILGVSPIGIQDHFFTLGGHSLKAIQLISRIQKECQADVPLRVLFEQPTIQALAAYVEGGEEGNVFSIEPVQKQAYYPVSSAQKRMYILDQFEGVGISYNMPSTMLIEGKLERTRVEAAFQRLIARHESLRTSFAVVNGEPVQNIHEDVPFALAYSEVTEQEARELVSSLVQPFDLEVAPLIRVSLLKIGEDRYVLFTDMHHSISDGVSSGILLAEWVQLYQGDVLPELRIQYKDFAVWQQEFSQSAAFHKQEAYWLQTFADDIPVLNLPTDFTRPSTQSFAGDQCTIGAGKALTEGLHQLAQATGTTLYMVLLAAYNVLLAKYAGQEDIIVGTPITGRSHADLEPIVGMFVNTLAMRNKPQREKTFSEFLQEVKQNALDAYGHQDYPFEELVEKLAIARDLSRNPLFDTVFTFQNSTEEVMTLPECTLAPFMTDETGQHAKFDLTFSATEEREEMTIGVEYSTSLFTRETMERFSRHFLTIAASIVQNPHIRLGEIDMLLPEEKQQILAGFNDTAVSYALDKTLHQLFEEQVDKTPDQAALLFSEQSLTYSELNERANRLARVLRAKGVGPDRLVAIMAERSPEMVIGILGILKAGGAYVPVDPGYPQERIQYLLEDSNAALLLSQAHLLPLLAQVSSELPECLDLNAELDAGLSGSNLPAVNQPTDLAYVIYTSGTTGKPKGVMIPHQGIVNCLQWRRDEYGFGPSDKALQVFSFAFDGFVASLFAPLLGGATCVLPQEAAAKDPVALKKLMAATEVTHYYGVPSLFQAILDCSTTTDFNQLRCVTLGGEKLPVQLVQKTKEKHPAIEINNEYGPTENSVVTTISRSIEAGQAITIGRPLANVQVYIVDEQHHLQPIGVVGELCIGGAGLARGYLNKPELTAEKFVANPFRPGERMYKTGDLVKWRTDGTIEYIGRADEQVKVRGYRIEIGEIESAVLAYQGIDQAVVVARDDDATAGSYLCAYFVAATAVSVSGLRSHLAKELPAYMIPSYFVELDQLPLSANGKVDRKALPKPQQSDATTREYVAPRNATEQQLAAIWQEVLGVEPIGITDQFFELGGHSLKATLLIAKVYEYMQIELPLNLIFQYPTIEKVADFITSEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 34 - protein, 4284 aa, Artificial Sequence
  NRPS synthesizing a Valine-Indigoidine-tagged Dipeptide consisting of Proline and Leucine. Valine is here used as spacer.
MDCVANNSGVELCQIPLLTEAETSQLLAKRTETAADYPAATMHELFSRQAEKTPEQVAVVFADQHLTYRELDEKSNQLARFLRKKGIGTGSLVGTLLDRSLDMIVGILGVLKAGGAFVPIDPELPAERIAYMLTHSRVPLVVTQNHLRAKVTTPTETIDINTAVIGEESRAPIESLNQPHDLFYIIYTSGTTGQPKGVMLEHRNMANLMHFTFDQTNIAFHEKVLQYTTCSFDVCYQEIFSTLLSGGQLYLITNELRRHVEKLFAFIQEKQISILSLPVSFLKFIFNEQDYAQSFPRCVKHIITAGEQLVVTHELQKYLRQHRVFLHNHYGPSETHVVTTCTMDPGQAIPELPPIGKPISNTGIYILDEGLQLKPEGIVGELYISGANVGRGYLHQPELTAEKFLDNPYQPGERMYRTGDLALWLPDGQLEFLGRIDHQVKIRGHRIELGEIESRLLNHPAIKEAVVIDRADETGGKFLCAYVVLQKALSDEEMRAYLAQALPEYMIPSFFVTLERIPVTPNGKTDRRALPKPEGSAKTKADYVAPTTELEQKLVAIWEQILGVSPIGIQDHFFTLGGHSLKAIQLISRIQKECQADVPLRVLFEQPTIQALAAYVEGGEEGNVFSIEPVQKQAYYPVSSAQKRMYILDQFEGVGISYNMPSTMLIEGKLERTRVEAAFQRLIARHESLRTSFAVVNGEPVQNIHEDVPFALAYSEVTEQEARELVSSLVQPFDLEVAPLIRVSLLKIGEDRYVLFTDMHHSISDGVSSGILLAEWVQLYQGDVLPELRIQYKDFAVWQQEFSQSAAFHKQEAYWLQTFADDIPVLNLPTDFTRPSTQSFAGDQCTIGAGKALTEGLHQLAQATGTTLYMVLLAAYNVLLAKYAGQEDIIVGTPITGRSHADLEPIVGMFVNTLAMRNKPQREKTFSEFLQEVKQNALDAYGHQDYPFEELVEKLAIARDLSRNPLFDTVFTFQNSTEEVMTLPECTLAPFMTDETGQHAKFDLTFSATEEREEMTIGVEYSTSLFTRETMERFSRHFLTIAASIVQNPHIRLGEIDMLLPEEKQQILAGFNDTAVSYALDKTLHQLFEEQVDKTPDQAALLFSEQSLTYSELNERANRLARVLRAKGVGPDRLVAIMAERSPEMVIGILGILKAGGAYVPVDPGYPQERIQYLLEDSNAALLLSQAHLLPLLAQVSSELPECLDLNAELDAGLSGSNLPAVNQPTDLAYVIYTSGTTGKPKGVMIPHQGIVNCLQWRRDEYGFGPSDKALQVFSFAFDGFVASLFAPLLGGATCVLPQEAAAKDPVALKKLMAATEVTHYYGVPSLFQAILDCSTTTDFNQLRCVTLGGEKLPVQLVQKTKEKHPAIEINNEYGPTENSVVTTISRSIEAGQAITIGRPLANVQVYIVDEQHHLQPIGVVGELCIGGAGLARGYLNKPELTAEKFVANPFRPGERMYKTGDLVKWRTDGTIEYIGRADEQVKVRGYRIEIGEIESAVLAYQGIDQAVVVARDDDATAGSYLCAYFVAATAVSVSGLRSHLAKELPAYMIPSYFVELDQLPLSANGKVDRKALPKPQQSDATTREYVAPRNATEQQLAAIWQEVLGVEPIGITDQFFELGGHSLKATLLIAKVYEYMQIELPLNLIFQYPTIEKVADFITTSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 35 - protein, 2168 aa, Artificial Sequence
  NRPS being a synthetase of a fusion peptide consisting of Valine and Indigoidine. Due to its sterical advantages, Valine may be used as a spacer between the indigoidine pigment and the NRPS oligopeptide of interest to be tagged with the pigment.
MYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKSSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 36 - protein, 3202 aa, Artificial Sequence
  NRPSase synthesizing a Indigoidine-tagged Dipeptide consisting of two Valine-monomers.
MYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHIATSGKETYVPIEPAPLQEYYPVSSAQKRMYVLRQFADTGTVYNMPSALYIEGDLDRKRFEAAIHGLVERHESLRTSFHTVNGEPVQRVHEHVELNVQYAEVTEAQVEPTVESFVQAFDLTKAPLLRVGLFKLAAKRHLFLLDMHHIISDGVSAGIIMEEFSKLYRGEELPALSVHYKDFAVWQSELFQSDVYTEHENYWLNAFSGDIPVLNLPADFSRPLTQSFEGDCVSFQADKALLDDLHKLAQESQSTLFMVLLAAYNVLLAKYSGQEDIVVGTPIAGRSHADIENVLGMFVNTLALRNYPVETKHFQAFLEEVKQNTLQAYAHQDYPFEALVEKLDIQRDLSRNPLFDTMFILQNLDQKAYELDGLKLEAYPAQAGNAKFDLTLEAHEDETGIHFALVYSTKLFQRESIERMAGHFLQVLRQVVADQATALREISLLSEEERRIVTVDFNNTFAYPRDLTIQELFEQQAAKTPEHAAVVMDGQMLTYRELNEKANQLAHVLRQNGVGKESIVGLLADRSLEMITGIMGILKAGGAYLGLDPEHPSERLAYMLEDGGVKVVLVQKHLLPLVGEGLMPIVLEEESLRPEDCGNPAIVNGASDLAYVMYTSGSTGKPKGVMVEHRNVTRLVMHTNYVQVRESDRMIQTGAIGFDAMTFEIFGALLHGASLYLVSKDVLLDAEKLGDFLRTNQITTMWLTSPLFNQLSQDNPAMFDSLRALIVGGEALSPKHINRVKSALPDLEIWNGYGPTENTTFSTCYLIEQHFEEQIPIGKPIANSTAYIVDGNNQPQPIGVPGELCVGGDGVARGYVNKPELTAEKFVPNPFAPGETMYRTGDLARWLPDGTIEYLGRIDQQVKIRGYRIELGEIETVLSQQAQVKEAVVAVIEEANGQKALCAYFVPEQAVDAAELREAMSKQLPGYMVPAYYVQMEKLPLTANGKVDRRALPQPSGERTTGSAFVAAQNDTEAKLQQIWQEVLGIPAIGIHDNFFEIGGHSLKAMNVITQVHKTFQVELPLKALFATPTIHELAAHISEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 37 - protein, 1591 aa, Artificial Sequence
  minimal construct C(of TycC2)-Ind
SEKTEYTAIQPVAAQEFYPVSSAQKRMYILQQFEGNGISYNISGAILLEGKLDYARFASAVQQLAERHEALRTSFHRIDGEPVQKVHEEVEVPLFMLEAPEDQAEKIMREFVRPFDLGVAPLMRTGLLKLGKDRHLFLLDMHHIISDGVSSQILLREFAELYQGADLQPLSLQYKDFAAWQNELFQTEAYKKQEQHWLNTFADEIPLLNLPTDYPRPSVQSFAGDLVLFAAGKELLERLQQVASETGTTLYMILLAAYNVLLSKYTGQEDIIVGTPVAGRSHADVENIMGIFVNTLALRNQPASSKTMLENNITQCDSINDVYLKEEAITLMDMLESQLKHQADGYVVIDQEESLSYADFYLRVKEIGYCLSEISSKSSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 38 - protein, 1949 aa, Shewanella violacea DSS12
MEPKSFNLAEQTSLVAVLQHRAQITPNKVAYIYLENGEDIEVPITYAELDCRARELAAQLQGKNPLIQQERVLLIYPQGIDFIVAFFATLYAGAIAVLVYPPSSKKMAQRLNGIVEDCNVKLILSTAKVISRMDRMNMVTDAGEQDEDAINIPAQYWINSDNLDPEAARDFKQPIILGEHLAFLQYTSGSTGTPKGVMISHSNLMANQAAIKDIYQHDDKTIFVGWLPLIHDMGLIGNVLQPMYLGISLVFMSPLHFVQKPVRWLRAISKYQATTSGGPNFAYDLCVRKIADADLADLDLSSWTLAYNGAEPVRKETVSRFNQRFSVCGLKPESHMAVYGLAEATLIVTGTNKQAVLATSDNVDYMSSGTCVEVDRVRIVNPETCVEADEQQEGEIWVHGPSVAKGYWNRPEETQTTFKAQILGSELHYMRTGDTGYCKNGEIHVTGRIKDIVIVQGKNFHPEDIEWSLIDVQGLRVGGSVAFSLDVVDEQGQTSESLVVVAGVLESDSDKHPSIISNIRSFIYQDHQLQVDRVVLIKPKQIPMTTSGKVQRRLTRQMLVANEFTILGDDLLAAVDDKSTQARSSIVAATTKAELELTSMWGAILGLSASDIGITDNFFDLGGSSLTMLELSIQLNTTMELLFRYPTISSYLYRTSEYEFPEVEKDIYLPAANIDRSLEGETGISLITGGTGFFGLHFLQSMMQRTQDKFVLLIRGENDDVMNKKFTDAVAYFHMEKDIDIGRVILIRGDLSEHHVGIPDDKYPWVCQNVDKIFHIGSHVNNWLPYEGIREINVDGTRSLLALARTGRKKEFHYTSTSTFSPDKADPSVFLEGDTIDKNDINRFFGYDISKYASEQMCRIAREEGLICNIYRLVWIGGHIETGLTKLNDGFNIMLRILITIKAFPKGNYLHDITPVDLLADGMASVQGKAKNTDFNLTSQSKESIDMKRLAVMLRGMGYQIDEVSRTEFVERLKNYPLEQWDEHCKSYRQLVIRLFEDPTPKIESFYDGSNFRKHVDPNLLVKMEQKFIDTWFEKTVNFLVSNNALPTPEGNVYDDEIKTLLTWGQHKGEFTHQQCIHHVFAQQVQRTPEAIAVRFNQDSLTYQELNERSEQVAQYLRNHAIAPGAVVGLCIERSTHLIVSILAIFKAGCAYLPLDPNYPAASLDHMIEDCAVKHILVANKSPQALVLHREKLISLTDVDFAMYAASELAPGISNTGQQSRPSDLAYVIYTSGTTGKPKGVQVEHRSVVNHSLSMADVFGLTGQDNVLQFSTINFDSFIEEVFPSLFTGATVVMIEQEKLTQVSELTELILQQSVNVVKFSTAYWHTVSKVNLQQLGVRLLAIGGEEADIQKYNEWRVINTDIPLINTYGPTETTVSASYSVLNGPLDNITIGRPIANTQAYILDSNLVPVAIGFVGELYIAGEGVSRGYLNNAELTAQVFIDNPFSGHSKMYKTGDLVRWDNAGNIEFMGRTDNQVKVRGYRIELGAIESVLNDYQGISQAVVVLKQIETKKKVVAYVVANNEAIDIAELGEHLSQALPSYMLPNLILPLDDIPLNPNGKVDRGLLEKMEINSEKSINFTSPVTDNEIKMTAIWQDVLAVSSVGLHDDFMELGGHSLLVMSLISEVNQEFNANVSINDIYESATVAKLLAVVENNDYEQGSNLVEFPNVHLSKTELTQVKPLFLVHGLGGHLASFYPLVKNLKQQLHDVYDIDIAVYGLEANGFKAQQQHFASVDEMVSEYIKLIKAKQASGPYLIGGWSYGVSIAYHIVQALINQGDEVEVFISIDAEAPYVPKDFAEFLRDNDVSGLNDLYQDEKLAALLKNFGKRFGFISNDKECIKQQFYRFLGYSQDDSQDQVERFNKVAIANLLNAKDFNPSTINPVNSLLVKASQSVFDDYVADWYDLLDSKMISLLTLTGDHWSIMQEQELASNLARVLAVSSQVVINES

SEQ ID NO: 39 - DNA, 5850 bp, Shewanella violacea DSS12
atggaacctaagtcgttcaacttagcggaacaaacatctttggttgctgttttacagcacagagcgcaaattacgccaaataaagttgcctatatttatttagaaaatggtgaagatattgaagtgcctatcacctacgctgaattagattgccgagctcgtgaactcgcggcgcaattacaagggaaaaacccactgattcagcaagagcgtgtgctactaatctatcctcaagggattgattttatagtggcattttttgccaccttgtacgcgggggcgatcgctgtgttggtgtatccacccagcagtaagaaaatggctcaacgcttaaatggcatagtcgaagattgtaacgtgaaattgattttatcgacggctaaagtgattagtcgtatggatcggatgaacatggtgaccgatgcaggcgaacaagatgaagatgccattaatatcccggcgcaatactggataaatagcgacaacttagatcctgaggcggccagggattttaagcagcctattattctaggtgagcatcttgcctttttacaatacacctccggctccacaggtactccaaaaggcgtgatgataagtcacagtaacttaatggccaaccaggccgcgatcaaggatatttatcaacatgacgacaaaacgatttttgtcggctggttgccgcttattcatgatatgggtctgattggtaatgtattacaacccatgtatttaggcatctccttggtgtttatgtcgccactgcatttcgtgcaaaaaccggtacgttggctacgtgctatcagtaagtatcaagcgaccaccagtggcggccctaattttgcctatgacttgtgtgtgcgaaaaatagccgatgctgatttggccgacttagacctatccagttggacgctggcatacaatggcgccgagcccgttcgcaaagaaactgtgagtcgttttaatcaaaggtttagcgtctgtgggctcaagcctgagtcgcatatggcggtatatggtttagccgaagccaccttaatcgtaaccggcaccaacaaacaagcggtattagccactagtgataatgtcgattatatgtcatctggaacatgtgttgaggtcgacagggtcagaattgttaaccctgaaacttgcgtcgaggctgatgagcaacaagagggcgaaatttgggtgcatggcccgagcgtagccaagggttattggaatcgcccagaagaaactcaaacgacttttaaggcgcagatcctcggcagcgagctgcattatatgcgcaccggtgatacaggttactgcaaaaatggtgaaatccatgtcacaggtcgtattaaagatatcgttatcgtgcaagggaaaaacttccacccagaggacatcgaatggagccttatcgatgtgcagggtctgcgagttggcggcagcgtggcattctcattagatgtggttgatgagcagggccaaaccagtgaatccttggtggttgtggcgggcgtattagagtcagatagtgacaagcaccccagcatcatcagtaatattcgctcgtttatctatcaagaccatcaattgcaagttgaccgtgtggtgctgattaaacctaagcaaatccccatgaccaccagtggcaaggtacagcgtcgtttaacccgtcaaatgttggtggccaatgaatttaccatccttggtgacgacctgttagcggctgtcgatgataaatcgactcaagccaggtctagtattgttgcagctaccaccaaagctgagctggaattaaccagtatgtggggcgcaatcttagggttatcggccagcgatatcggcatcacagataacttctttgatttaggtggttcctcattgaccatgttggagctatcaattcagttaaataccaccatggagctgttatttcgctacccaactattagttcatatttatatcgcactagcgagtatgagtttccagaagtcgagaaagatatctatttaccggcagccaatatagacaggagtttagaaggtgaaactggtattagcttgatcaccggtggtactggattctttggcttacattttctgcaaagtatgatgcagcgtacccaggacaaatttgttttgttaattcgtggcgaaaatgatgacgtcatgaacaaaaagtttaccgatgcagtggcttatttccatatggaaaaagacatagatataggcagagtgatcttaattaggggggatttaagtgagcaccatgtaggtattcctgatgataagtacccttgggtttgccagaatgtggataagattttccatatcggctcccatgtcaataactggctcccctatgaaggcatacgcgagatcaatgtcgatggcactcggagcttattggcgcttgctcgtaccggacgtaagaaggagttccactataccagtaccagtactttctcaccggataaagccgatccgtctgtgttcctagaaggcgatactatcgataaaaacgatatcaatcgtttctttggttatgacataagtaaatatgccagtgagcaaatgtgccgtattgctagagaagaagggcttatttgtaatatctatcgtttggtctggataggcggtcatatcgagaccgggctaactaagctcaacgatggctttaatattatgctgcgtattttaatcaccattaaagcctttcctaagggaaattatctccacgatattaccccggtagatctattggctgatggtatggcatcggtgcaaggtaaagccaaaaataccgactttaacttaaccagtcagtcgaaagaatccatcgacatgaaacgtttagccgtgatgttgcgtggcatgggttatcaaatcgatgaggtgagtcgtaccgaatttgttgagcgtctaaaaaattacccattggagcaatgggatgagcattgtaagtcgtaccgccaactggtgatccgcttatttgaagaccccacgcctaaaatagaatctttttatgatggtagtaacttcagaaagcatgttgatccaaacttgctggttaagatggagcaaaaattcatcgatacctggttcgaaaagacggtcaacttcttagtcagtaataatgccctgcctacaccggaggggaatgtttatgatgatgaaattaagaccttattgacctggggccagcataagggtgagttcacacatcaacaatgtatacaccatgtatttgcccaacaagtacaaagaaccccagaggcgattgcggttaggtttaatcaagacagtttaacctatcaggagttgaatgagcgtagcgagcaagtagcccaatacttgcgtaatcatgccattgcccccggtgctgtggtgggcttatgtatcgagcgttccacacacttgattgtatccatcttggccatcttcaaagccggttgcgcctatttaccattggaccctaattatcccgccgcgagtctggatcatatgatagaagactgcgccgttaagcatattttagtggccaataagtcgccacaagcactagtgcttcatcgggaaaagctgatttcactgaccgatgttgactttgccatgtacgcggccagtgaattagctcccggcatatcaaatactggccagcaatcacggccgagtgatctggcctatgtgatttacacttcgggcaccacaggcaagcctaaaggggtacaggttgagcataggagtgtggtgaatcacagtttaagtatggctgatgtgtttggtttgactggacaagataatgtattacagttctcaaccatcaactttgattcttttatcgaagaagtgtttcccagcttatttactggcgctactgtggtgatgattgagcaggagaagcttacccaagtgagcgagctaactgagttaattctccagcagtcggtcaacgtggttaagttctccaccgcctactggcacactgtgtctaaggttaacttgcagcaactgggtgtgcgattgttagccatagggggtgaagaggccgatattcagaaatacaatgagtggcgagtcattaataccgatattccccttatcaacacctatgggccaactgagacgacagtgagcgccagttactcagtattaaatggtccgctcgataacatcaccataggccggccaattgccaatacccaagcttacatcttggacagtaacttggttcctgtggccattggctttgtgggtgaactctatattgctggtgaaggggtcagtcggggttatctcaataatgccgagcttaccgcgcaagtgtttattgataatccttttagcggtcattctaagatgtataaaacaggggatctggtacgttgggacaatgccggtaatattgagtttatgggccgcacagacaaccaggtgaaagttcgcggttatcgtatcgagctcggcgccattgaaagtgtgttaaatgactatcaaggtattagccaggccgtggtagtgctgaagcaaattgaaaccaagaagaaagtggttgcctatgttgtggccaataatgaggcgattgatattgccgagctaggggagcatctatcccaagccttgcctagttatatgctgcctaatctaatattacctctcgatgatattcctctcaatcccaacggcaaagttgatcgtggcttgctagaaaagatggagattaatagtgagaaaagtattaatttcacctctccggtgacggataatgaaatcaaaatgacggccatttggcaagatgtattggcggtatcgagtgtcggtttacatgatgacttcatggagcttggtggccactcattgctagttatgtcgcttataagtgaagtgaaccaagagtttaatgctaatgtcagtatcaatgatatttatgagtcggcgacggttgccaagttactcgccgtggtcgaaaataatgactatgagcaagggtctaatttggttgaatttcccaacgttcacctctctaagactgagttaactcaggttaaacctctgttcttagtccatggtctaggggggcatctagcgtctttctatcccttggtgaagaacttaaagcagcagttacatgatgtgtatgatattgatattgcagtttatggcctagaagccaatggttttaaggctcagcagcaacactttgccagtgtcgatgagatggtgagtgaatacattaaactgataaaggctaagcaggcatcgggcccatacctgataggtggctggtcttatggcgtctcgattgcttaccacatagtgcaagcgctcattaatcagggcgatgaagtcgaggtgtttatctccatagatgctgaggcaccctatgtgccaaaagactttgcagagttcttgcgagacaatgatgtctctggtttgaatgacttatatcaggatgaaaaactggcggcgctgttgaaaaacttcggcaaacgttttggctttatcagtaatgacaaagagtgtattaagcagcagttttatcgctttttaggctattcacaagatgatagtcaagaccaagtcgagcgcttcaataaggtggccatagccaatctgttaaatgctaaggactttaaccccagcacaattaacccggttaattcgctcttagttaaagcatcacagagtgtcttcgatgattacgtcgccgattggtatgacttactcgacagtaagatgatatcactgcttactttaaccggagatcattggtccattatgcaggagcaagaattggcaagtaatttagcaagagtactcgctgttagctcacaggtggtaattaacgagagctag

(19) United States
(12) Patent Application Publication
(10) Pub. No.: US 2016/0238611 A1
(43) Pub. Date: Aug. 18, 2016

(54) NON-RIBOSOMAL PROTEIN SYNTHESIS PIGMENT FUSION PEPTIDES

(71) Applicants: Deutsches Krebsforschungszentrum, Heidelberg (DE); Ruprecht-Karls-Universität Heidelberg, Heidelberg (DE)

(72) Inventors: Roland EILS, Schriesheim (DE); Barbara DI VENTURA, Heidelberg (DE); Lorenz ADLUNG, Grosfahner (DE); Katharina GENREITH, Heidelberg (DE); Tim HEINEMANN, Dossenheim (DE); Hannah MEYER, St. Wendel (DE); Dominik NIOPEK, Heidelberg (DE); Fanny GEORGI, Dresden (DE); Ralf BEER, Opfingen (DE); Tania Christina CHRISTIANSEN, Wiesloch (DE); Konrad HERBST, Beelitz Ortsteil Fichtenwalde (DE); Nikolaos IGNATIADIS, Heidelberg (DE); Ilia KATS, Heidelberg (DE); Nils KURZAWA, Friedrichshafen (DE); Johanna MEICHSNER, Heidelberg (DE); Sophie RABE, Heidelberg (DE); Anja RIEDEL, Heidelberg (DE); Joshua SACHS, Langen (DE); Julia Patricia SCHESSNER, Heidelberg (DE); Florian SCHMIDT, Armsheim (DE); Philipp Darius Konstantin WALCH, Heidelberg (DE)

(21) Appl. No.: 15/026,734

(22) PCT Filed: Oct. 2, 2014
(86) PCT No.: PCT/EP2014/071155

§ 371 Date: Apr. 1, 2016

(30) Foreign Application Priority Data
Oct. 2, 2013 (EP) 13187133.7

(51) Int. Cl. G01N 33/58; C12Q 1/02; C12N 9/16; C12P 21/00; C07K 2/00; C12N 9/00

(52) U.S. Cl. CPC G01N 33/583; C07K 2/00; C12N 9/93; C12N 9/16; C12P 21/00; C12Q 1/02; C12Y 603/02; C12Y 301/00; G01N 2458/00; G01N 2440/00

(57) ABSTRACT

The present invention relates to a polypeptide or polypeptide complex, comprising at least one non-ribosomal peptide synthesis (NRPS) amino acid module functionally connected to at least one pigment module. The present invention further relates to a labeled oligopeptide comprising a non-naturally attached NRPS pigment or/and polyketide pigment, to a polynucleotide encoding a fusion polypeptide, a vector, preferably an expression vector, comprising the polynucleotide of the present invention and to a host cell comprising the polypeptide or polypeptide complex and/or the polynucleotide, and/or the vector according to the present invention. Moreover, the present invention relates to in vitro and in vivo method of producing a labeled oligopeptide, as well as to methods of optimizing the same.

The present invention relates to a polypeptide or polypeptide complex, comprising at least one non-ribosomal peptide synthesis (NRPS) amino acid module functionally connected to at least one pigment module. The present invention further relates to a labeled oligopeptide comprising a non-naturally attached NRPS pigment or/and polyketide pigment, to a polynucleotide encoding a fusion polypeptide, a vector, preferably an expression vector, comprising the polynucleotide of the present invention and to a host cell comprising the polypeptide or polypeptide complex and/or the polynucleotide, and/or the vector according to the present invention. Moreover, the present invention relates to in vitro and in vivo method of producing a labeled oligopeptide, as well as to methods of optimizing the same.

Non-ribosomal peptides (NRPs) are secondary metabolites produced by microorganisms, e.g. bacteria and fungi. Unlike ribosomal protein biosynthesis, non-ribosomal protein synthesis (NRPS) does not require mRNA to direct the sequence of monomers incorporated into the growing peptide chain. In NRPS, this sequence is controlled by the sequence of amino acid modules within the enzyme, the non-ribosomal peptide synthetase (NRPSase), wherein each module is specific for one amino acid, respectively.

Every NRPS module contains a Thiolation-domain (T-domain), also called PCP-domain (Peptidyl-carrier-protein-domain). In the synthesis of non-ribosomal peptides, growing peptide chains are handed from one module to the next one. A new amino acid is first adenylated by the A-domain and then bound to the T-domain via a thioester bond. The C-domain catalyzes the condensation of an existing peptide chain—which is bound to the T-domain of the previous module—and the amino acid of the next module. The T-domain itself does not exhibit any substrate specificity but is just a carrier domain to keep the peptide attached to the NRPS module complex. The core of every T-domain is a conserved 4′-phosphopanthetheinylated (4′-PPT) serine. The 4′-PPT residue is added by a 4′-Phosphopanthetheinyl-transferase (PPTase), which brings the NRPS apo-enzyme to its active holo-form.

NRPSases have been known to have a modular architecture, with single modules being specific for a specific amino acid as described above. The single modules can be connected covalently to form large multi-module-proteins. However, single modules can also associate via protein-protein interactions, mostly via so called communication domains. Both, in covalently connected modules as well as in non-covalently connected modules, it was shown that single modules can generally be exchanged for modules having different specificities, and still the NRPSase is functional. Also, it was found to be generally possible to exchange domains comprised in a module for another domain conferring the same functionality, emphasizing the modular architecture of NRPSases on several levels. These approaches have been used to create new NRPSases synthesizing novel peptides (Mootz et al (2000), PNAS 97(11):5848-53; Nguyen et al. (2006), PNAS 103(46):17462-7; Stachelhaus et al. (1998), J Biol Chem 273(35):22773-81; Finking & Marahiel (2004), Annu Rev Mibrobiol 58:453-88).

Indigoidine is an insoluble blue pigment probably formed by oxidation of two cyclic glutamines. There exist several enzymes catalyzing indigoidine synthesis, all of which are NRPSases and consist of only one single module with an A-Ox- (adenylation and oxidation), T- and a TE-domain.

Previous publications showed that exchanging the T-domain of the indigoidine synthetase bpsA from S. lavendulae with other T-domains results in a loss of function, i.e. the indigoidine synthetase loses its ability to produce the blue pigment (Owen et al. (2012), Environ Microbiol 14: 1198-1209). In the same study a method is described, in which the T-domain of the E. coli entF gene was inserted into bpsA and afterwards modified using a random mutagenesis strategy, until blue colonies were obtained. Studies of Marahiel and Doekel (Doekel & Marahiel (2000, Chem Biol 7(6):373-84) showed that it is possible to exchange the A-domains of NRPS modules to yield modified nonribosomal peptide products for some module combinations.

However, peptide production via NRPSases has been hampered by the fact that the peptides have to be purified from the reaction mixture in order to be able to evaluate the amount and the quality of the product. Thus, NRPS at present is amenable to high-throughput methods only to a very limited extent. Accordingly, there is a need in the art for improved methods of allowing detection of peptides produced by NRPS. This Problem is solved by the embodiments of the present invention described herein.

Accordingly, the present invention relates to a polypeptide or polypeptide complex, comprising at least one non-ribosomal peptide synthesis (NRPS) amino acid module functionally connected to at least one pigment module.

The term “oligopeptide”, as used herein, relates to a chemical compound comprising at least one peptide bond. Preferably, the oligopeptide of the present invention comprises at least one alpha-amino acid involved in a peptide bond. Preferably, the oligopeptide is a non-ribosomal peptide synthesis (NRPS) oligopeptide, i.e. an oligopeptide synthesized by an NRPSase as described herein. Preferably, the oligopeptide comprises 2 to 25 amino acid units, more preferably 3 to 20 amino acid units, most preferably 5 to 15 amino acid units. It is understood by the skilled person that in NRPS, amino acids as well as derivatives thereof, including short peptides, and other chemical compounds may be added to the growing oligopeptide chain; moreover, said compounds integrated may subsequently be modified chemically, e.g. by oxidation, reduction, or isomerization, e.g. epimerization. Accordingly, the term amino acid unit, as used herein, relates to a subpart of the oligopeptide, which is incorporated in a condensation step of NRPS as described elsewhere in this specification. Preferably, the oligopeptide is a peptide, a natural NRPS oligopeptide or a derivative thereof; also preferably, the oligopeptide is a non-natural NRPS peptide generated by module-shuffling as described elsewhere herein, or a derivative thereof.

The term “non-ribosomal peptide synthesis” or “NRPS” is known in the art and relates to the formation of at least one peptide bond in the absence of polynucleotides, preferably mRNA, catalyzed by a polypeptide or polypeptide complex as defined herein below. Preferably, NRPS is the enzymatically catalyzed condensation of at least two amino acid units. More preferably, NRPS is the synthesis of a peptide by one of a group of specific enzymes known as non-ribosomal peptide synthetases (NRPSases).

As used herein, the term “NRPS amino acid module” relates to a subpart of a NRPSase catalyzing at least activation of an amino acid and condensation thereof to the growing oligopeptide chain. Preferably, the NRPS amino acid module comprises at least one condensation domain, at least one adenylation domain, and at least one thiolation domain. Examples for NRPS amino acid modules and domains comprised therein are well known in the art. Preferably, the NRPS amino acid module is specific for activation of a specific amino acid. NRPS amino acid modules have been covered extensively by databases, e.g. “A database of NonRibosomal Peptide Synthetases” at the New Delhi National Institute of Immunology” by M. Z. Ansari, R. S. Gokhale, and D. Mohanty (http://linux1.nii.res.in/˜zeeshan/webpages/home.html), “NORINE”: Caboche et al. (2008), Nucleic Acids Res. 36 (Database issue): D326-31 (http://bioinfo.lifl.fr/norine/), and “ClusterMine360”: Conwayet al. (2013), Nucleic Acids Res. 41 (Database issue): D402-7, (http://www.clusteimine360.ca/). Preferably, the NRPS amino acid module is an NRPS amino acid module as described herein. As it was detailed herein above, NRPSases are structured modularly, both in comprising modules typically catalyzing the addition of one amino acid unit, and in that each module itself comprises specific domains. Thus, preferably, the term NRPS amino acid module also includes to non-natural modules, preferably including synthetic modules. Also preferably, the NRPS amino acid module of the present invention may comprise one or more non-natural domain(s), more preferably one or more synthetic domains(s).

The term “pigment”, as used herein, relates to a chemical compound having at least one absorption maximum at a wavelength of visible light. Preferably, the at least one absorption maximum is at a wavelength between 380 nm and 750 nm, more preferably between 400 nm and 650 nm. Methods of determining absorption maxima of chemical compounds are known in the art. Preferably, the pigment is synthesized as a pro-pigment, requiring further, spontaneous or catalyzed, modification, including, preferably, oxidation, cyclisation, or aggregation. Also preferably, the pigment is synthesized as an active pigment, i.e., preferably, as a pigment having said absorption maximum. Preferably, the pigment is a pigment generated by a polyketide synthase, i.e. a polyketide pigment. More preferably, the pigment is a pigment generated by a non-ribosomal peptide synthetase, i.e. the pigment is an NRPS pigment.

The term “NRPS pigment”, as used herein, relates to a chemical compound generated at least in part by NRPS having at least one absorption maximum at a wavelength of visible light. Preferably, the NRPS pigment comprises at least two amino acid units. Preferably, the at least one absorption maximum is at a wavelength between 380 nm and 750 nm, more preferably between 400 nm and 650 nm. Methods of determining absorption maxima of chemical compounds are known in the art. Preferably, the NRPS pigment is synthesized as a pro-pigment in the NRPS process, requiring further, spontaneous or catalyzed, modification, including, preferably, oxidation, cyclisation, or aggregation. Also preferably, the NRPS pigment is synthesized as an active pigment, i.e., preferably, as a pigment having said absorption maximum, in the NRPS. Preferably, the NRPS pigment is actinomycin (Schauwecker et al. (1998), J Bacteriol. 180(9): 2468-74). More preferably, the NRPS pigment is indigoidine (IUPAC Name: (5E)-3-amino-5-(5-amino-2,6-dioxopyridin-3-ylidene)pyridine-2,6-dione, CAS Registry Number: 2435-59-8). Preferably, the term NRPS pigment relates to the complete pigment molecule as it is produced by a naturally occurring NRPS pigment module.

The term “pigment module” relates to a polypeptide or polypeptide complex catalyzing the synthesis of a pigment according to the present invention. Preferably, the pigment module is a polyketide synthase module. More preferably, the pigment module is an NRPS pigment module.

The term “NRPS pigment module” relates to a polypeptide or polypeptide complex contributing to the synthesis of an NRPS pigment. Preferably, the NRPS pigment module is a polypeptide or polypeptide complex catalyzing the synthesis of an NRPS pigment. More preferably, the NRPS pigment module is a polypeptide catalyzing indigoidine synthesis, e.g. an indigoidine synthetase as shown in Table 1, more preferably the NRPS pigment module is the indigoidine synthetase encoded by the indC gene of Photorhabdus luminescens (gene: SEQ ID NO: 1, protein: SEQ ID NO: 25). Preferably, the term NRPS pigment module also includes to non-natural modules, preferably including synthetic modules. Also preferably, the NRPS pigment module of the present invention may comprise one or more non-natural domain(s), more preferably one or more synthetic domains(s).

TABLE 1
Indigoidine Synthetases

| | synonym | | | |
|---|---|---|---|---|
| | and | | | |
| name | description | NCBI Acc No | source organism | reference |
| indC | hypothetical | NP_929446.1 | Photorhabdus luminescens subsp. | Duchaud, E. et. al., Nat. Biotechnol. 21 (11), 1307-1313 |
| (SEQ ID | protein | GI: 37526102 | laumondii TTO1 | (2003), The genome sequence of the entomopathogenic |
| NO: 25) | plu2186 | | | bacterium Photorhabdus luminescens |
| | | | | Brachmann AO et. al. (2012) Triggering the production of |
| | | | | the cryptic blue pigment indigoidine from Photorhabdus |
| | | | | luminescens. J Biotechnol 157: 96-99. |
| | indigoidine | WP_017892269.1 | Serratia sp. S4 | |
| | synthase | GI: 516503831 | | |
| | indigoidine | WP_017237530.1 | Streptomyces sp. SS | |
| | synthase | GI: 515806777 | | |
| indC | putative | YP_004349727.1 | Burkholderia gladioli BSR3 | Seo, Y. S. et. al., Complete Genome Sequence of |
| | indigoidine | GI: 330820865 | | Burkholderia gladioli BSR3, J. Bacterial. 193 (12), 3149 |
| | synthase | | | (2011) |
| indC | putative | ACK77757.1 | Streptomyces aureofaciens | Novakova, R. et. al., Identification and characterization of an |
| | indigoidine | GI: 218511496 | | indigoidine-like gene for a blue pigment biosynthesis in |
| | synthase sa8 | | | Streptomyces aureofaciens CCM 3239, Folia Microbiol. |
| | | | | (Praha) 55 (2), 119-125 (2010) |
| bpsA | Blue-pigment | BAE93896.1 | Streptomyces lavendulae subsp. | Takahashi, H. et. al., Cloning and Characterization of a |
| | synthetase | GI: 94467513 | lavendulae | Streptomyces Single Module Type Non-ribosomal Peptide |
| | | | | Synthetase Catalyzing a Blue Pigment Synthesis, J, Biol. |
| | | | | Chem. 282 (12), 9073-9081 (2007) |
| | Blue-pigment | YP_007934704.1 | Streptomyces fulvissimus DSM | Myronovskyi, M., et. al. Complete genome sequence of |
| | synthetase | GI: 488613368 | 40593 | Streptomyces fulvissimus, Submitted (03-APR-2013) |
| | igiD, indC, | | | Helmholtz Institute for Pharmaceutical Research Saarland, |
| | bpsA, sa8 | | | Helmholtz Center for Infectious Research, University |
| | | | | Campus, Building C 2.3, Saarbrucken, Saarland 66123, |
| | | | | Germany |
| | blue-pigment | WP_007269003.1 | Streptomyces sp. C | |
| | synthetase | GI: 494479526 | | |
| indC | Putative | WP_003963722.1 | Streptomyces clavuligerus | |
| | indigoidine | GI: 490061478 | | |
| | synthase | | | |
| indC | blue-pigment | WP_003952690.1 | Streptomyces clavuligerus | |
| | synthetase | GI: 490050342 | | |
| | indigoidine | WP_016941560.1 | Dickeya zeae | |
| | synthase | GI: 515508306 | | |
| | indigoidine | WP_019435944.1 | Streptomyces sp. AA0539 | |
| | synthase | GI: 518265736 | | |
| | indigoidine | WP_019844163.1 | Dickeya zeae | |
| | synthase | GI: 518682470 | | |
| indC | indigoidine | AFV27434.1 | Streptomyces chromofuscus | Yu, D. et. al. An indigoidine biosynthetic gene cluster from |
| | synthase | GI: 409183839 | (ATCC 49982) | Streptomyces chromofuscus ATCC 49982 contains an |
| | | | | unusual IndB homologue, J. Ind. Microbiol. Biotechnol. 40 |
| | | | | (1), 159-168 (2013) |
| | indigoidine | YP_003885171.1 | Dickeya dadantii 3937 | Glasner, J. D. et. al., Genome Sequence of the |
| | synthase | GI: 307133155 | | Plant-Pathogenic Bacterium Dickeya dadantii 3937, J. |
| | | | | Bacteriol. 193 (8), 2076-2077 (2011) |
| indC | indigoidine | CAB87990.1 | Erwinia chrysanthemi | Reverchon, S. et. al., Characterization of indigoidine |
| | synthase | GI: 7576265 | | biosynthetic genes in Erwinia chrysanthemi and role of this |
| | | | | blue pigment in pathogenicity, J. Bacteriol. 184 (3), |
| | | | | 654-665 (2002) |
| | Putative | YP_007526476.1 | Streptomyces davawensis JCM | Jankowitsch, F., et. al., Genome Sequence of the Bacterium |
| | indigoidine | GI: 471327446 | 4913 | Streptomyces davawensis JCM 4913 and |
| | synthase | | | Heterologous Production of the Unique Antibiotic |
| | | | | Roseoflavin, J. Bacteriol. 194 (24), 6818-6827 (2012) |
| | indigoidine | WP_010472001.1 | Streptomyces somaliensis | |
| | synthase | GI: 498157845 | | |
| | indigoidine | WP_018512405.1 | Streptomyces sp. ScaeMP-e10 | |
| | synthase | GI: 517336913 | | |
| | Blue-pigment | YP_007931314.1 | Streptomyces fulvissimus DSM | Myronovskyi, M. et. al., Complete genome sequence of |
| | synthetase, | GI: 488609978 | 40593 | Streptomyces fulvissimus, Submitted (03-APR-2013) |
| | igiD, indC, | | | Helmholtz Institute for Pharmaceutical Research Saarland, |
| | bpsA, sa8 | | | Helmholtz Center for infectious Research, University |
| | | | | Campus, Building C 2.3, Saarbrucken, Saarland 66123, |
| | | | | Germany |
| | indigoidine | WP_018894040.1 | Streptomyces sp. CNY228 | |
| | synthase | GI: 517723832 | | |
| | Blue-pigment | YP_007748943.1 | Streptomyces albus J1074 | Rabyk, M. et. al., Complete genome analysis and |
| | synthetase | GI: 478692133 | | transcriptional profile of Streptomyces albus J1074, |
| | | | | Submitted (25-FEB-2013) Department of Genetics and |
| | | | | Biotechnology, Ivan Franko National University of Lviv, |
| | | | | Hrushevskyy str., 4, Lviv 79005, Ukraine |
| | indigoidine | WP_018488639.1 | Streptomyces sp. CcalMP-8W | |
| | synthase | GI: 517299821 | | |
| | indigoidine | WP_018471088.1 | Streptomyces sp. LaPpAH-202 | |
| | synthase | GI: 517282270 | | |
| | blue-pigment | WP_003946752.1 | Streptomyces albus | |
| | synthetase | GI: 490044390 | | |
| igiD | | AAD54007.1 | Vogesella indigofera | van de Loo, F. J., et. al., Structural and regulatory genes |
| | | GI: 5852326 | | controlling indigoidine production in Vogesella indigofera: |
| | | | | involvement of a peptide synthetase homolog, Submitted |
| | | | | (01-SEP-1998) Plant Industry, CSIRO, G.P.O. Box 1600, |
| | | | | Canberra, ACT 2601, Australia |
| | blue pigment | AFT64148.1 | alpha proteobacterium U95 | Penesyan, A. et. al., Assessing the effectiveness of |
| | synthetase | GI: 407188354 | | functional genetic screens for the identification of bioactive |
| | | | | metabolites, Mar Drugs 11 (1), 40-49 (2013) |
| | indigoidine | YP_002546883.1 | Agrobacterium radiobacter K84 | Setubal, J. et. al., Genome sequencing of three |
| | synthase, | GI: 222102293 | | Agrobacterium biovars illustrates the role of gene flow |
| | Arad_12458 | | | among plasmids and chromosomes in the evolution of |
| | | | | pathogenic and symbiotic alpha proteobacteria, J. Bacteriol. |
| | | | | (2009) |
| indC | Putative | WP_010110104.1 | Verminephrobacter aporrectodeae | |
| | indigoidine | GI: 497795920 | | |
| | synthase, | | | |
| | partial | | | |
| | indigoidine | YP_001624984.1 | Renibacterium salmoninarum | Wiens, G. D., et. al., Genome sequence of the fish pathogen |
| | synthase | GI: 163840579 | ATCC 33209 | Renibacterium salmoninarum suggests reductive evolution |
| | | | | away from an environmental Arthrobacter ancestor, J. |
| | | | | Bacteriol. 190 (21), 6970-6982 (2008) |
| indC | indigoidine | CAD27331.1 | Erwinia chrysanthemi | Reverchon, S., vfm genes of Erwinia chrysanthemi |
| | synthase, | GI: 19571812 | (Pectobacterium chrysanthemi = | modulate the synthesis of multiple virulence factors, |
| | partial | | Dickeya chrysanthemi) | Submitted (05-MAR-2002) Reverchon S., Biochimie, Institut |
| | | | | des Sciences Appliquees de Lyon, 11 avenue Jean Capelle, |
| | | | | Villeurbanne 69621, FRANCE |
| | indigoidine | | Streptomyces laurentii | Tala Mubadda Suidan, Mining the cryptic non ribosomal |
| | synthetase, | | ATCC 31255 | peptide systems of Streptomyces laurentii, BSc. thesis, |
| | NRPS2, | | | Georgia Institute of Technology, Dec. 2010; Wendy L. K. et. |
| | bpsA/indC | | | al., Thiostrepton Biosynthesis: Prototype for a New Family |
| | homolog | | | of Bacteriocins, J. Am. Chem. Soc., 2009, 131 (12), pp |
| | | | | 4327-4334 |
| igiD | indigoidine | RBY4I_2890 | Roseobacter Phaeobacter sp. | Cude, W. N., Mooney, J., Tavanaei, A. a, Hadden, M. K., |
| | synthetase | | Strain Y4I | Frank, A. M., Gulvik, C. a, . . . Buchan, A. (2012). Production |
| | | | | of the antimicrobial secondary metabolite indigoidine |
| | | | | contributes to competitive surface colonization by the |
| | | | | marine roseobacter Phaeobacter sp. strain Y4I. Applied and |
| | | | | environmental microbiology, 78(14), 4771-80 |

The term “polypeptide”, as used herein, relates to a macromolecule comprising at least the modules and/or domains as defined herein. Preferably, the polypeptide comprises a contiguous chain of peptide bonds forming the backbone of the polypeptide. More preferably, the polypeptide comprises a contiguous chain of alpha-amino acids interconnected by peptide bonds. Even more preferably, the polypeptide is synthesized by in vitro protein biosynthesis; most preferably, the polypeptide is synthesized by in vivo protein biosynthesis. In the context of the present invention, the term polypeptide, preferably, relates to a polypeptide of between 50 and 30000 amino acids in length comprising at least one domain of an NRPS amino acid module and/or at least one domain of an NRPS pigment module. The polypeptide may comprise further amino acids which may serve as a tag for purification or detection, as a linker, or as a communication domain. In a preferred embodiment of the polypeptide of the present invention, said polypeptide further comprises a detectable tag. The term “detectable tag” refers to a stretch of amino acids which are added to or introduced into the polypeptide of the invention. Preferably, the tag shall be added C- or N-terminally to the polypeptide. The said stretch of amino acids shall allow for detection of the polypeptide by an antibody which specifically recognizes the tag or it shall allow for forming a functional conformation, such as a chelator or it shall allow for visualization by fluorescent tags. Preferred tags are the Myc-tag, FLAG-tag, 6-His-tag, HA-tag, GST-tag or GFP-tag. These tags are all well known in the art. The term polypeptide also includes chemically modified polypeptides, e.g., containing modified amino acids or being biotinylated or coupled to fluorophores, such as fluorescein, or Cy 3, being conformationally restricted, e.g. by disulfide bridging or by stapling (Walensky 2004, Science 305(5689): 1466-1470), or being linked to cell penetration polypeptides or protein transduction domains (Snyder 2004, Pharm Res 21(3): 389-393). Such modifications may improve the biological properties of the polypeptide, e.g., complex formation, binding, stability, or may be used as detection labels.

As used herein, the term polypeptide, preferably, also relates to a variant of a polypeptide, including variants characterized by one or more amino acid exchanges or deletions or additions of amino acids. The term, preferably, also includes fragments of the polypeptides specifically mentioned, provided that said polypeptide variants still have an activity as detailed herein.

Preferably, the NRPS amino acid module or modules and the pigment module or modules are comprised in a fusion polypeptide catalyzing the synthesis of a pigment, more preferably an NRPS pigment, covalently connected to the amino acid activated by the NRPS amino acid module. More preferably, at least two NRPS amino acid modules and a pigment module are comprised in a fusion polypeptide catalyzing the synthesis of a pigment covalently connected to the oligopeptide synthesized by the NRPS amino acid modules.

Accordingly, the term “polypeptide complex” relates to a complex comprising at least two polypeptides of the present invention. In such case, the polypeptides comprised in the polypeptide complex may be referred to as subunits of the complex. Preferably, polypeptides of the polypeptide complex are connected by a chemical linkage. It is envisaged by the present invention that the chemical bond between the subunits is an ester bond, a disulfide bond, or any other suitable covalent chemical bond known to the skilled artisan. More preferably, the subunits are connected via non-covalent bonds with a dissociation constant so low that the subunits will only dissociate to a negligible extent. Preferably, the dissociation constant for said non-covalent bond is less than 10⁻⁵ mol/l (as it is the case with the Strep-Tag:Strep-Tactin binding), less than 10⁻⁶ mol/l (as it is the case in the Strep-TagII:Strep-Tactin binding), less than 10⁻⁸ mol/1, less than 10⁻¹⁰ mot/1, or less than 10⁻¹² mol/l (as it is the case for the Streptavidin:Biotin binding). Methods of determining dissociation constants are well known to the skilled artisan and include, e.g., spectroscopic titration methods, surface plasmon resonance measurements, equilibrium dialysis and the like. In a preferred embodiment, the polypeptide consists of the components as described herein. Most preferably, at least one of the polypeptides of the polypeptide complex comprises a communication domain of an NRPS amino acid domain known in the art.

Preferably, the polypeptide or polypeptide complex of the present invention comprises a fusion polypeptide comprising at least a NRPS amino acid module condensation domain and a NRPS pigment module adenylation domain. Preferably, the polypeptide or polypeptide complex of present invention is a polypeptide or polypeptide complex comprising the C domain of the at least one NRPS amino acid module and the at least one NRPS pigment module as a fusion polypeptide. It is understood by the one skilled in the art that in such case, preferably, the polypeptide or polypeptide complex comprises the adenylation domain and the thiolation domain as further components of a fusion polypeptide; more preferably, the adenylation domain and the thiolation domain are comprised as a subunit or as subunits of a polypeptide complex. More preferably, the polypeptide or polypeptide complex is a polypeptide or polypeptide complex comprising the at least one NRPS amino acid module and the at least one NRPS pigment module as a fusion polypeptide; preferably, the NRPS amino acid module comprises at least a condensation domain, an adenylation domain, and a thiolation domain as detailed herein above. Preferably, the polypeptide or polypeptide complex comprises at least one NRPS amino acid module. More preferably, the polypeptide or polypeptide complex comprises at least two NRPS amino acid modules. Most preferably, the polypeptide or polypeptide complex comprises the number of NRPS amino acid module corresponding to the number of amino acid units comprised in the oligopeptide to be synthesized. Since a given NRPS amino acid module is specific for a specific amino acid unit, it will be appreciated that the selection of the specific NRPS amino acid module or NRPS amino acid modules and their sequence in the polypeptide or polypeptide complex determines the sequence of amino acid units in the oligopeptide produced. The NRPS pigment module can, in principle, be located within the polypeptide or polypeptide complex at any location deemed appropriate. Accordingly, the NRPS pigment may be included at any position within the oligopeptide. Preferably, the NRPS pigment module is functionally connected to the NRPS module mediating the last elongation step. Accordingly, preferably, the NRPS pigment is the last unit synthesized onto the oligopeptide. Preferably, the NRPS amino acid module preceding the pigment module is specific for a small, neutral amino acid; more preferably, the NRPS amino acid module preceding the pigment module is specific for glycine, alanine, or valine.

Preferably, the polypeptide or polypeptide complex of the present invention comprises NRPS amino acid modules in an arrangement as it is found in nature. Accordingly, preferably, the polypeptide or polypeptide complex catalyzes the synthesis of a labeled natural NRPS oligopeptide. More preferably, the polypeptide or polypeptide complex comprises NRPS amino acid modules in an arrangement not found in nature. Accordingly, more preferably, polypeptide or polypeptide complex catalyzes the synthesis of a labeled non-natural NRPS oligopeptide.

The term “functionally connected” is understood by the skilled person. Preferably, the term relates to two polypeptides of the present invention being connected in a way that enables transfer of a growing oligopeptide chain from one polypeptide of a polypeptide complex to the other, thus permitting condensation of at least one further amino acid unit onto the oligopeptide after transfer. Preferably, functional connection is mediated by protein-protein interaction, more preferably by protein-protein interaction between a communication domain and a polypeptide or between two communication domains present on two different polypeptides. More preferably, functional connection is mediated by covalent interconnection of two polypeptides. Most preferably, functional connection is achieved by combining two polypeptides as a fusion polypeptide by methods well known to the skilled person and as described herein.

Advantageously, it was surprisingly found in the work underlying the present invention that an NRPS pigment module can be functionally connected to a non-ribosomal peptide synthetase, resulting in the production of labeled NRPS peptides. As will be appreciated, these tools are especially useful in, e.g., tracking or determining the amount of NRPS oligopeptides. Moreover, the tools are useful in the optimization of in vitro or in vivo NRPS peptide production, since they allow for convenient identification of producer cells and for convenient determination of the amount of NRPS oligopeptide produced, e.g. semiquantitatively by thin layer chromatography (TLC) or quantitatively by photometric measurement of culture coloring as described herein in the Examples.

The definitions made above apply mutatis mutandis to the following. Additional definitions and explanations made further below also apply for all embodiments described in this specification mutatis mutandis.

The present invention further relates to a labeled oligopeptide comprising a non-naturally attached NRPS pigment or/and polyketide pigment.

As used herein, the term “labeled oligopeptide” relates to an oligopeptide as described herein comprising covalently attached at least one NRPS pigment or/and at least one polyketide pigment. Preferably, the NRPS pigment is a non-naturally attached NRPS pigment, i.e., preferably, an NRPS pigment not known to be covalently attached to said oligopeptide in nature. Also preferably, the polyketide pigment is a non-naturally attached polyketide pigment, i.e., preferably, a polyketide pigment not known to be covalently attached to said oligopeptide in nature. More preferably, the labeled oligopeptide is an indigoidine-labeled oligopeptide. i.e. the NRPS pigment comprised in the labeled oligopeptide is indigoidine.

Moreover, the present invention relates to a polynucleotide encoding a fusion polypeptide according to the present invention.

The term “polynucleotide”, as used in accordance with the present invention, relates to a polynucleotide comprising a nucleic acid sequence which encodes a fusion polypeptide as described herein above having the activity of catalyzing the condensation of an amino acid unit activated by an NRPS amino acid module adenylation domain with a second amino acid unit activated by an NRPS pigment module adenylation domain. Suitable assays for measuring the activities mentioned before are described in the accompanying Examples or in Owen et al. (2011), The Biochemical journal, 436(3): 709-17; Owen et al. (2012), Environmental microbiology, 14(5): 1198-209; Müller et al. (2012), Metabolic engineering, 14(4): 325-35; Takahashi et al. (2007), JBC 282(12): 9073-9081; Myers et al. (2013), BMC Biophysics, 6(1): 4).

The polynucleotide, preferably, comprises the nucleic acid sequence shown in SEQ ID NO: 11, 13-19, 23, 24, or, more preferably, SEQ ID NO:26 encoding fusion polypeptides of SEQ ID NOs: 27, 28-34, 35, 36, and 37, respectively. It is to be understood that a fusion polypeptide encoded by one of the aforementioned polynucleotides may be also encoded due to the degenerated genetic code by other polynucleotides as well.

Moreover, the term “polynucleotide” as used in accordance with the present invention further encompasses variants of the aforementioned specific polynucleotides. Said variants may represent orthologs, paralogs or other homologs of the polynucleotide of the present invention. The polynucleotide variants, preferably, comprise a nucleic acid sequence characterized in that the sequence can be derived from the aforementioned specific nucleic acid sequences by at least one nucleotide substitution, addition and/or deletion whereby the variant nucleic acid sequence shall still encode a fusion polypeptide having the activity as specified above. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N.Y. (1989), 6.3.1-6.3.6. A preferred example for stringent hybridization conditions are hybridization conditions in 6′ sodium chloride/sodium citrate (=SSC) at approximately 45° C., followed by one or more wash steps in 0.2′ SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5′ SSC (pH 7.2). If organic solvent is present in the abovementioned buffer, for example 50% formamide, the temperature under standard conditions is approximately 42° C. The hybridization conditions for DNA:DNA hybrids are preferably for example 0.1′ SSC and 20° C. to 45° C., preferably between 30° C. and 45° C. The hybridization conditions for DNA:RNA hybrids are preferably, for example, 0.1′ SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the fusion polypeptides of the present invention. Conserved domains of the fusion polypeptide of the present invention may be identified by a sequence comparison of the nucleic acid sequence of the polynucleotide or the amino acid sequence of the fusion polypeptide of the present invention with sequences of other non-ribosomal peptide synthetases or non-ribosomal pigment synthetases. Oligonucleotides suitable as PCR primers as well as suitable PCR conditions are described in the accompanying Examples. As a template, DNA or cDNA from bacteria, fungi, plants or animals may be used. Further, variants include polynucleotides comprising nucleic acid sequences which are at least 70%, at least 75%, at least 80%, at least 85%, at least 90%, at least 95%, at least 98% or at least 99% identical to the nucleic acid sequences detailed herein. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences of the fusion polypeptides of the present invention. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software packet [Genetics Computer Group, 575 Science Drive, Madison, Wis., USA 53711 (1991)], are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the aforementioned nucleic acid sequences, preferably, is also encompassed as a polynucleotide of the present invention. The fragment shall encode a fusion polypeptide which still has the activity as specified above. Accordingly, the fusion polypeptide may comprise or consist of the domains of the polypeptide of the present invention conferring the said biological activity. A fragment as meant herein, preferably, comprises at least 50, at least 100, at least 250 or at least 500 consecutive nucleotides of any one of the aforementioned nucleic acid sequences or encodes an amino acid sequence comprising at least 20, at least 30, at least 50, at least 80, at least 100 or at least 150 consecutive amino acids of any one of the aforementioned amino acid sequences.

The polynucleotides of the present invention either essentially consist of the aforementioned nucleic acid sequences or comprise the aforementioned nucleic acid sequences. Thus, they may contain further nucleic acid sequences as well. The fusion proteins encoded may comprise as additional part other enzymes, polypeptides for monitoring expression (e.g., green, yellow, blue or red fluorescent proteins, alkaline phosphatase and the like) or so called “tags” which may serve as a detectable marker or as an auxiliary measure for purification purposes as detailed elsewhere herein.

The polynucleotide of the present invention shall be provided, preferably, either as an isolated polynucleotide (i.e. isolated from its natural context) or in genetically modified form. The polynucleotide, preferably, is DNA, including cDNA, or RNA. The term encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

The present invention also relates to a vector comprising the polynucleotide according the present invention.

The term “vector”, preferably, encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination. The vector encompassing the polynucleotide of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells.

More preferably, in the vector of the invention the polynucleotide is operatively linked to expression control sequences allowing expression in prokaryotic or eukaryotic cells or isolated fractions thereof. Expression of said polynucleotide comprises transcription of the polynucleotide, preferably into a translatable mRNA. Regulatory elements ensuring expression in prokaryotic or eukaryotic cells are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (InVitrogene) or pSPORT1 (GIBCO BRL). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population.

Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The present invention further relates to a host cell comprising the polypeptide or polypeptide complex and/or the polynucleotide and/or the vector according to the present invention.

The term “host cell”, as used herein, relates to any bacterial, archeal, or eukaryotic cell. Preferably, the cell is a eukaryotic cell, more preferably an insect or mammalian cell; more preferably, the eukaryotic cell is a fungal cell, most preferably a yeast cell, e.g. a cell of Saccharomyces cerevisiae. More preferably, the cell is bacterial cell, even more preferably an Escherichia cell (e.g. E. coli) or a Bacillus cell (e.g. B. subtilis) (Zhang et al. (2011), Nat Prod Rep 28: 125-151). Preferably, the host cell further comprises a broad-specificity phosphopantetheinyl transferase (PPTase), more preferably an Sfp-type PPTase.

The following methods of the present invention may, preferably, comprise further steps in addition to those explicitly mentioned. E.g., in all methods, a further step may be detecting or determining the amount of labeled oligopeptide. Further examples of preferred additional steps are described for the respective methods. Moreover, one or more of the steps of the methods of the present invention may be performed by automated equipment.

The present invention also relates to an in vitro method of producing a labeled oligopeptide, comprising:
a) incubating a polypeptide or polypeptide complex according to present invention with appropriate amino acid substrates,
b) thereby producing a labeled oligopeptide.

The present invention further relates to a method for optimizing in vitro production of a labeled oligopeptide by improving incubation conditions, comprising
a) incubating the polypeptide or polypeptide complex according the present invention under modified conditions suspected to improve production of said labeled oligopeptide,
b) comparing the amount of labeled oligopeptide produced to the amount produced by said polypeptide or polypeptide complex under unmodified conditions, and, thereby
c) optimizing in vitro production of a labeled oligopeptide.

Moreover, the present invention relates to a method for optimizing in vitro production of a labeled oligopeptide, comprising
a) incubating a variant of a polypeptide or polypeptide complex according to the present invention under conditions suitable for production of said labeled oligopeptide,
b) comparing the amount of labeled oligopeptide produced to the amount produced by the unmodified polypeptide or polypeptide complex, and, thereby
c) optimizing in vitro production of a labeled oligopeptide.

Further steps preferably included in the in vitro methods of the present invention may relate, e.g., to including further compounds in the incubation step, or to purifying the labeled oligopeptide. It is understood that the definitions below apply to variants of the polypeptides or polypeptide complexes mutatis mutandis.

The term “incubating the polypeptide or polypeptide complex”, as used in the context of the in vitro methods of the present invention, is understood by the skilled person. Preferably, the term relates to bringing a polypeptide or polypeptide complex according to present invention in physical contact with amino acids and thereby, e.g. allowing the polypeptide or polypeptide complex and the amino acids to interact. As will be appreciated, incubating, preferably, relates to mixing the polypeptide or polypeptide complex of the present invention with at least the other components as defined herein in an appropriate solvent under appropriate conditions, e.g. temperature. Appropriate solvents, preferably, are water-based buffers known in the art. As will also be appreciated, the incubation mixture may, preferably, comprise further ingredients, e.g. an energy source, e.g. ATP, or ions required by the polypeptide or polypeptide complex of the invention, e.g. magnesium ions. Preferably, the composition of the incubation solution is one of the compositions known in the art, (e.g. in Doekel & Marahiel (2000), Chem. Biol. 7(6): 373-84; Stein et al. (2006), Chembiochem 7(11): 1807-14; Owen et al. (2011), Biochem. J. 436(3): 709-17). Preferably, the term relates to maintaining the polypeptide or polypeptide complex under conditions allowing production of labeled oligopeptide by said polypeptide or polypeptide complex. Appropriate conditions, including, without limitation, buffers, ion concentrations, temperature, and the like, depend on the polypeptide or polypeptide complex selected and are known in the art. The summary of the conditions used in incubating a host cell is known as “incubation conditions”. Thus, “modified incubation conditions”, as used herein, relates to incubation conditions wherein at least one parameter has been modified as compared to the incubation conditions used before optimization. Accordingly, the term “improving incubation conditions” relates to modifying one more incubation parameters for a polypeptide or polypeptide complex such that the production of labeled oligopeptide is improved.

The term “amino acid substrate” relates to a chemical compound corresponding to the amino acid unit activated by a NRPS amino acid module or pigment module included in the polypeptide or polypeptide complex used. Accordingly, appropriate amino acid substrates are the compounds corresponding to the amino acid units activated by the NRPS amino acid modules or pigment modules included in the polypeptide or polypeptide complex used in the method.

The term “optimizing” as used herein, relates to improving the yield of labeled oligopeptide obtainable by a process. Preferably, the term relates to improving the yield of full-length oligopeptide.

“Comparing,” as used herein, relates to a comparison of corresponding parameters or values, e.g., an absolute amount is compared to an absolute reference amount; a concentration compared to a reference concentration; an intensity signal obtained is compared to the same type of intensity signal. The comparison referred to in the methods of the present invention may be carried out manually or computer assisted. For a computer assisted comparison, the value of the determined amount or ratio may be compared to values corresponding to suitable references which are stored in a database by a computer program. The computer program may further evaluate the result of the comparison by means of an expert system. Accordingly, the result of the comparison referred to herein may be automatically provided in a suitable output format.

The term “conditions suspected to improve production”, as used herein, preferably relates to any conditions not known not to improve production. As will be appreciated by the skilled person, factors affecting in vitro production of oligopeptides are difficult to predict.

Accordingly, in principle, any modification of incubation conditions is suspected to improve production, unless it is known that this is not the case. More preferably, incubation conditions are suspected to improve production if said improvement appears to be a reasonable conclusion in view of what is known on the production process, or if an improvement was found by applying said conditions in a similar case.

Moreover, the present invention relates to an in vivo method of producing a labeled oligopeptide, comprising:
a) incubating a host cell comprising the polypeptide or polypeptide complex and/or the expression vector according to the present invention,
b) thereby producing a labeled oligopeptide.

Also, the present invention relates to a method for optimizing in vivo production of a labeled oligopeptide by improving culture conditions, comprising
a) incubating a host cell comprising the polypeptide or polypeptide complex and/or the expression vector according to the present invention under modified conditions suspected to improve production of said labeled oligopeptide,
b) comparing the amount of labeled oligopeptide produced to the amount produced by said host cell under unmodified conditions, and, thereby
c) optimizing in vivo production of a labeled oligopeptide.

Further, the present invention relates to a method for optimizing in vivo production of a labeled oligopeptide, comprising
a) incubating a host cell comprising a polypeptide or polypeptide complex variant and/or an expression vector comprising a polynucleotide variant according to the present invention under conditions suitable for production of said labeled oligopeptide,
b) comparing the amount of labeled oligopeptide produced to the amount produced by a host cell comprising an unmodified polypeptide or polypeptide complex and/or an unmodified expression vector according the present invention, and, thereby
c) optimizing in vivo production of a labeled oligopeptide.

The term “incubating a host cell”, as used in the context of the in vivo methods of the present invention, is understood by the skilled person. Preferably, the term relates to maintaining the host cell comprising the polypeptide or polypeptide complex and/or the expression vector under conditions allowing proliferation of said host cell and/or production of labeled oligopeptide by said host cell. Appropriate conditions, including, without limitation, medium, temperature, oxygen and/or carbon dioxide tension, and the like, depend on the host cell selected and are known in the art. The summary of the conditions used in incubating a host cell is known as “culture conditions”. Thus, “modified culture conditions”, as used herein, relates to culture conditions wherein at least one parameter has been modified as compared to the culture conditions used before optimization. Accordingly, the term “improving culture conditions” relates to modifying one more incubation parameters for a host cell such that the production of labeled oligopeptide is improved.

The term “conditions suspected to improve production”, as used herein, preferably relates to any conditions not known not to improve production. As will be appreciated by the skilled person, factors affecting in vivo production of oligopeptides are difficult to predict. Accordingly, in principle, any modification of culture conditions is suspected to improve production, unless it is known that this is not the case. More preferably, culture conditions are suspected to improve production if it said improvement appears a reasonable conclusion in view what is known on the production process, or if an improvement was found by applying said conditions in a similar case.

The present invention further relates to a labeled oligopeptide obtainable by one of the methods of producing a labeled oligopeptide according to the present invention.

The present invention also relates to a kit for in vivo synthesis of a labeled oligopeptide comprising an expression vector according to of the present invention and an expression vector encoding at least one further NRPS amino acid module.

Moreover, the present invention relates to a kit for in vitro synthesis of a labeled oligopeptide comprising a polypeptide or polypeptide complex according to the present invention and at least one further NRPS amino acid module.

The term “kit”, as used herein, refers to a collection of the aforementioned means, preferably, provided separately or within a single container. The container, also preferably, comprises instructions for carrying out the method of the present invention. The components of the kit are provided, preferably, in a “ready-to-use” manner, e.g., concentrations are adjusted accordingly, etc.

Summarizing the findings of the present invention, the following embodiments are preferred:

### EMBODIMENT 1

A Polypeptide or Polypeptide Complex, Comprising at Least One Non-ribosomal peptide synthesis (NRPS) amino acid module functionally connected to at least one pigment module.

### EMBODIMENT 2

The polypeptide or polypeptide complex of embodiment 1, wherein the at least one NRPS amino acid module and the at least one pigment module are comprised in a fusion polypeptide catalyzing the synthesis of a pigment covalently connected to the amino acid activated by the NRPS amino acid module.

### EMBODIMENT 3

The polypeptide or polypeptide complex of embodiment 1 or 2, comprising the C domain of the at least one NRPS amino acid module and the at least one pigment module as a fusion polypeptide.

### EMBODIMENT 4

The polypeptide or polypeptide complex of any one of embodiments 1 to 3, comprising the at least one NRPS amino acid module and the at least one pigment module as a fusion polypeptide.

### EMBODIMENT 5

The polypeptide or polypeptide complex of any one of embodiments 1 to 4, wherein the polypeptide or polypeptide complex comprises at least two NRPS amino acid modules.

### EMBODIMENT 6

The polypeptide or polypeptide complex of embodiment 5, wherein the pigment module is functionally connected to the NRPS module mediating the last elongation step.

### EMBODIMENT 7

The polypeptide or polypeptide complex of any one of embodiments 1 to 6, wherein the pigment module is an NRPS pigment module.

### EMBODIMENT 8

The polypeptide or polypeptide complex of any one of embodiments 1 to 7, wherein the pigment module is an indigoidine synthetase.

### EMBODIMENT 9

The polypeptide or polypeptide complex of any one of embodiments 1 to 8, wherein the pigment module is an indigoidine synthetase encoded by one of the genes specified in Table 1, preferably the indC gene of Photorhabdus luminescens, more preferably the indC gene of Photorhabdus luminescens laumondii TT01.

### EMBODIMENT 10

The polypeptide or polypeptide complex of any one of embodiments 1 to 9, wherein the NRPS amino acid module or the NRPS amino acid modules is/are selected from the NRPS amino acid modules encoded by any one of SEQ ID NO: 11 to 24.

### EMBODIMENT 11

A labeled oligopeptide comprising a non-naturally attached NRPS pigment or/and polyketide pigment.

### EMBODIMENT 12

The labeled oligopeptide of embodiment 10, wherein the pigment is indigoidine.

### EMBODIMENT 13

A polynucleotide encoding a fusion polypeptide according to any one of embodiments 3 to 10.

### EMBODIMENT 14

A vector comprising the polynucleotide according to embodiment 13.

### EMBODIMENT 15

The vector of embodiment 14, wherein the vector is an expression vector.

### EMBODIMENT 16

A host cell comprising the polypeptide or polypeptide complex according to any one of embodiments 1 to 10 and/or the polynucleotide according to embodiment 13, and/or the vector according to embodiment 14 or 15.

### EMBODIMENT 17

An in vitro method of producing a labeled oligopeptide, comprising:
a) incubating a polypeptide or polypeptide complex according to any one of embodiments 1 to 10 with appropriate amino acid substrates,
b) thereby producing a labeled oligopeptide.

### EMBODIMENT 18

An in vivo method of producing a labeled oligopeptide, comprising:
a) incubating a host cell comprising the polypeptide or polypeptide complex according to any one of embodiments 1 to 10 and/or the expression vector according to embodiment 15,
b) thereby producing a labeled oligopeptide.

### EMBODIMENT 19

A labeled oligopeptide obtainable by the method according to embodiment 17 or by the method according to embodiment 18.

### EMBODIMENT 20

A method for optimizing in vivo production of a labeled oligopeptide by improving culture conditions, comprising
a) incubating a host cell comprising the polypeptide or polypeptide complex according to any one of embodiments 1 to 10 and/or the expression vector according to embodiment 15 under modified conditions suspected to improve production of said labeled oligopeptide,
b) comparing the amount of labeled oligopeptide produced to the amount produced by said host cell under unmodified conditions, and, thereby
c) optimizing in vivo production of a labeled oligopeptide.

### EMBODIMENT 21

A method for optimizing in vitro production of a labeled oligopeptide by improving incubation conditions, comprising
a) incubating the polypeptide or polypeptide complex according to any one of embodiments 1 to 10 under modified conditions suspected to improve production of said labeled oligopeptide,
b) comparing the amount of labeled oligopeptide produced to the amount produced by said polypeptide or polypeptide complex under unmodified conditions, and, thereby
c) optimizing in vitro production of a labeled oligopeptide.

### EMBODIMENT 22

A kit for in vivo synthesis of a labeled oligopeptide comprising an expression vector according to embodiment 15 and an expression vector encoding at least one further NRPS amino acid module.

### EMBODIMENT 23

A kit for in vitro synthesis of a labeled oligopeptide comprising a polypeptide or polypeptide complex according to any one of embodiments 1 to 10 and at least one further NRPS amino acid module.

### EMBODIMENT 24

A method for optimizing in vivo production of a labeled oligopeptide, comprising
a) incubating a host cell comprising a polypeptide or polypeptide complex variant and/or an expression vector comprising a polynucleotide variant according to the present invention under conditions suitable for production of said labeled oligopeptide,
b) comparing the amount of labeled oligopeptide produced to the amount produced by a host cell comprising an unmodified polypeptide or polypeptide complex according to any one of embodiments 1 to 10 and/or an unmodified expression vector according to embodiment 15, and, thereby
c) optimizing in vivo production of a labeled oligopeptide.

### EMBODIMENT 25

A method for optimizing in vitro production of a labeled oligopeptide, comprising
a) incubating a variant of a polypeptide or polypeptide complex according to any one of embodiments 1 to 10 under conditions suitable for production of said labeled oligopeptide,
b) comparing the amount of labeled oligopeptide produced to the amount produced by the unmodified polypeptide or polypeptide complex, and, thereby
c) optimizing in vitro production of a labeled oligopeptide.

All references cited in this specification are herewith incorporated by reference with respect to their entire disclosure content and the disclosure content specifically mentioned in this specification.

### FIGURE LEGENDS

FIG. 1: Constructs encoding fusion NRPSs consisting of one module from the Tyrocidine-cluster (incorporating the indicated amino acids), a linker C-domain and the Indigoidine-module. NRPS expression was driven from an IPTG-inducible promoter.

FIG. 2: Restriction digest (EcoRI) of three different NRPSs that are putative synthetases for respective Indigoidine-fusion peptides.

FIG. 3: SDS-PAGE of three different NRP Ss that are putative synthetases for respective Indigoidine-fusion peptides. Annotation: pPW03=Phe-Ind, pPW04=Asn-Ind, pPW05=Val-Ind. Arrows indicate the position the expected bands indicating expression of the different synthetic NRPSs.

FIG. 4: Comparison of Valine-Indigoidine fusion NRP and Indigoidine-control by Thin Layer Chromatography (TLC). 3 different biological replicates of the fusion peptide and technical replicates of the indigoidine control are shown, alongside with the DiMethylSulfoxid (DMSO)-control. Note: All samples were solved in DMSO during the purification prior to the TLC.

FIG. 5: TLC of three technical replicates of Orn-Val-Ind with two technical Indigoidine controls. Orn-Val-Ind shows a slower migration behavior compared to Indigoidine control on silica-gel with Dichloromethane as mobile phase.

FIG. 6: Quantitative indigoidine assay by Optical Density measurements. For quantitative assessment of indigoidine production OD values at two wavelengths are considered: the sensitive OD value at 590 nm is superposed by the absorption by cell matter as well as indigoidine accumulating in the culture media. For this reason a second robust OD value is taken at 800 nm which is only affected by absorption from cell matter. a) Absorption spectra for a positive indigoidine producing strain of TOP10 carrying the indigoidine synthetase indC_T12 (pRB23 T12) and the PPTase sfp (pRB15) as well as for two different types of negative controls: TOP10 without additional plasmids and with an unfunctional indigoidine synthetase indC_T16 (pRB23 T16) and sfp. The arrow marks a peak between 550 nm and 650 nm for the indigoidine producing stain. b) When the sensitive OD at 590 is analyzed with respect to the robust OD at 800 nm. For non indigoidine producing control strains TOP10 and TOP10 with pRB23_T16 and with pRB15 these values depend proportionally on each other. Thereby a specific delta value can be derived which is used for the calculation of the indigoidine produced. c) The derived concentration of indigoidine in the inspected culture volume over time is shown. Both negative results show no indigoidine production whereas TOP10 with pRB23_T12 and pRB15 show a increase in indigoidine within the first 16.5 h after incubation leading to a steady state. All plots are derived from measurements taken under similar conditions (37° C., Luria Broth media, 200 μl culture volume).

The following Examples shall merely illustrate the invention. They shall not be construed, whatsoever, to limit the scope of the invention.

### EXAMPLE 1

### Generation of NRPS Peptide—Indigoidine Fusions

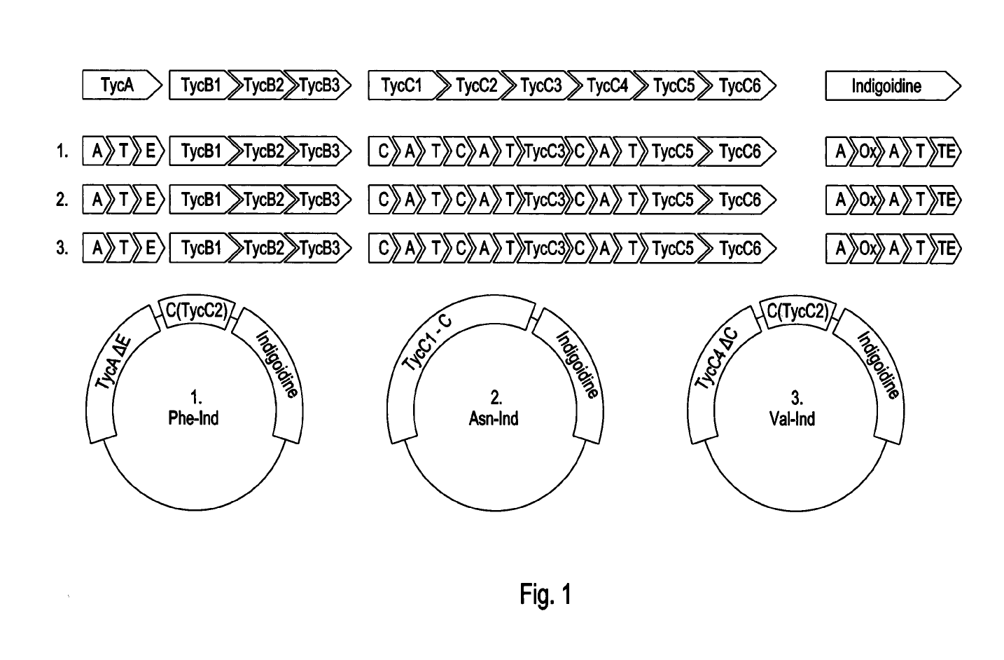

In order to create fusion-NRPSs consisting of a module derived from the Tyrocidine-cluster and the IndC-module that is responsible for the formation of Indigoidine, we assembled the three constructs as depicted in FIG. 1.

Some days after Gibson Assembly of the constructs and Transformation of E. coli BAP1-cells (BAP1 cells express the Sfp, a PPTase with broad substrate specificity necessary for NRPS activation) with the NRPS-expressing plasmids, we could detect blue colonies on our plates for all three different synthetic NRPS expression constructs (Phe-Ind, Asn-Ind and Val-Ind); Occurance of blue colonies directly on plates without any IPTG-induction is due to the leakiness of the IPTG-inducible promoter used for driving NRPS expression). Successful assemblies of NRPS-expressing plasmids present in the blue colonies were further confirmed by restriction digest and sequencing.

### A) Validation of the Genotype

All constructs were digested with EcoRI, which should lead to fragments of the following expected sizes:
Phe-Ind: 5858 and 3183
Asn-Ind: 5858 and 3582
Val-Ind: 5858 and 3105 Restriction digest was carried out with 1 μl enzyme (EcoRI from New England Biolabs) and 200 ng to 1000 ng DNA per reaction, reaction volume was 20 μl with 2 μl 10× CutSmart buffer from New England Biolabs. Samples were mixed with 4 μl 6× loading dye and electrophoresis was conducted in 0.8% agarose gel, with 10 μl Ethidiumbromide, at 100V for 40 minutes. As standard, 1 kb+ gene ruler from Fermentas was used.

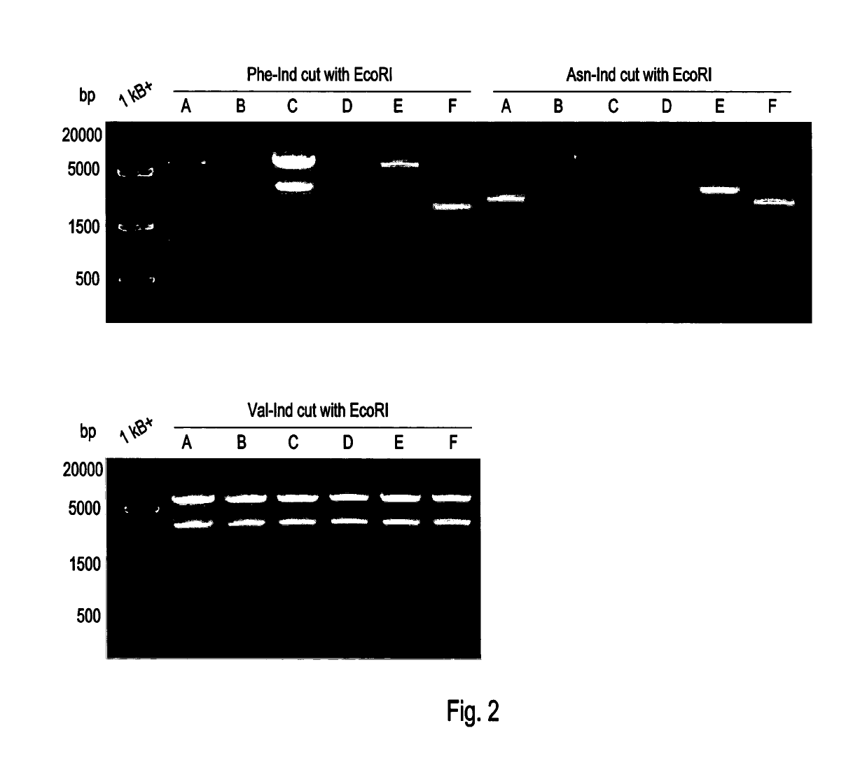

As one can clearly see in FIG. 2, clones A, B, C and D for Phe-Ind, samples B, C and D for Asn-Ind and all samples for Val-Ind show the expected restriction pattern.

### B) Validation of Expression

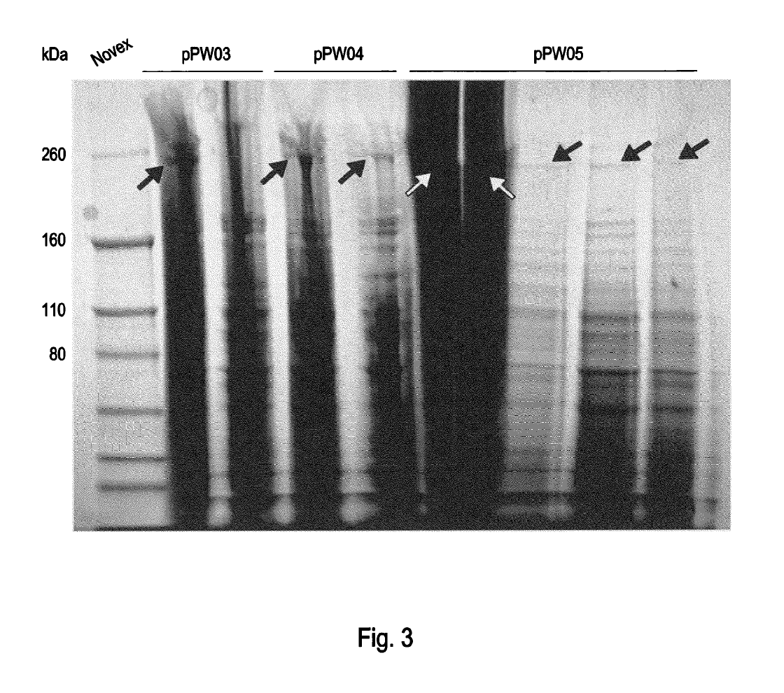

Blue colonies were subsequently inoculated in LB media and NRPS-expression was induced using 1 mM IPTG. Expression of the full-length, fusion NRPS-Protein was confirmed by SDS-PAGE followed by coomassie staining. FIG. 3 shows the coomassie staining for the fusion-NRPSs that are capable of synthesizing the Indigoidine-labelled NRPs. According to calculated protein mass, the expected full-length-NRPS bands would appear at (IndC alone has a size of 145 kDa):
Phe-Ind-NRPS: 247 kDa
Asn-Ind-NRPS: 261 kDa
Val-Ind-NRPS: 242 kDa

As the gel in FIG. 3 shows, the obtained bands match well with what has been expected. As pPWO5 containing clones showed both, the clearest band at the expected size on the SDS page indicating successful expression of the Val-IndC NRPS as well as a dark-blue coloring of the corresponding liquid culture, we analyzed these clones further.

C) Characterization of the Synthetic Val-Ind NRPS Encoded on pPW05

As mentioned beforehand, liquid cultures containing BAP1 cells expressing the Val-Ind fusion NRPS turned blue after induction with 1 mM IPTG. On plates colonies turned blue after several days without induction, due to leaky expression of the IPTG inducible promoter.

In order to show that NRPS expression and NRP production can be performed in different E. coli strains, we transformed the pPW05 plasmid in TOP10 cells and co-expressed the Sfp plasmid in trans from a separate construct.

Finally, we wanted to show that Indigoidine labeled NRPs can easily be characterized by thin-layer chromatography in order to show functioning of the corresponding, synthetic NRPS.

Therefore, the Valine-Indigoidine NRP was purified from BAP1 cells. 1 ml of induced, blue culture was therefore spun down at full speed (14,000 rpm) for 20 minutes. The blue pellet was washed in 1 ml of methanol and centrifuged once more for 5 minutes at 14,000 rpm. Methanol was discarded and the blue pellet dissolved in 200-400 μl DMSO (Yu et al. (2013), J Ind Microbiol Biotechnol 40: 159-168).

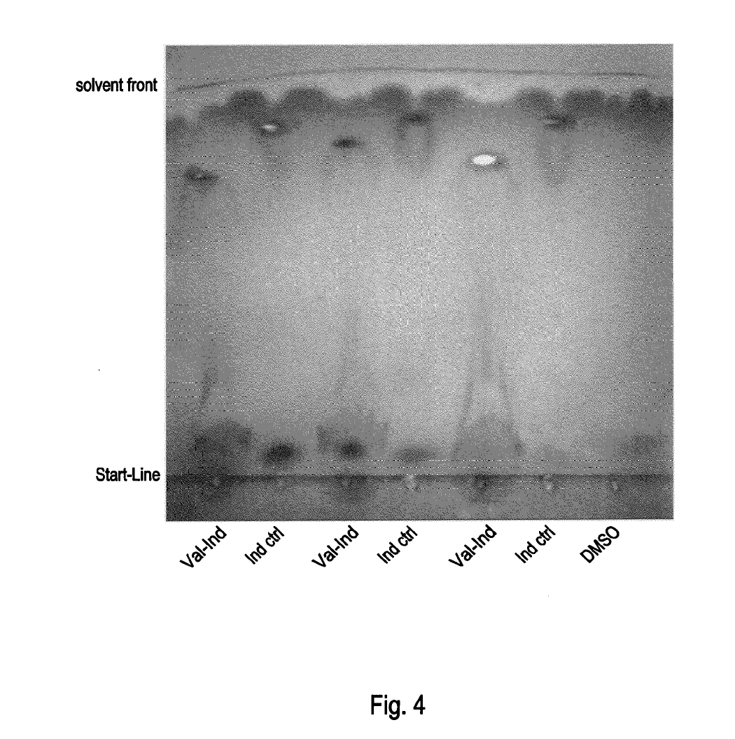

Valine-Indigoidine has a bigger mass than indigoidine alone, the Valine-Indigoidine fusion NRP is expected to migrate slower on the TLC. Using three different biological replicates of the produced NRP and technical replicates of an Indigoidine control (FIG. 4), we could show that the produced NRP indeed shows a slower migration behavior.

D) Confirmation with an Orn-Val-Ind Oligopeptide

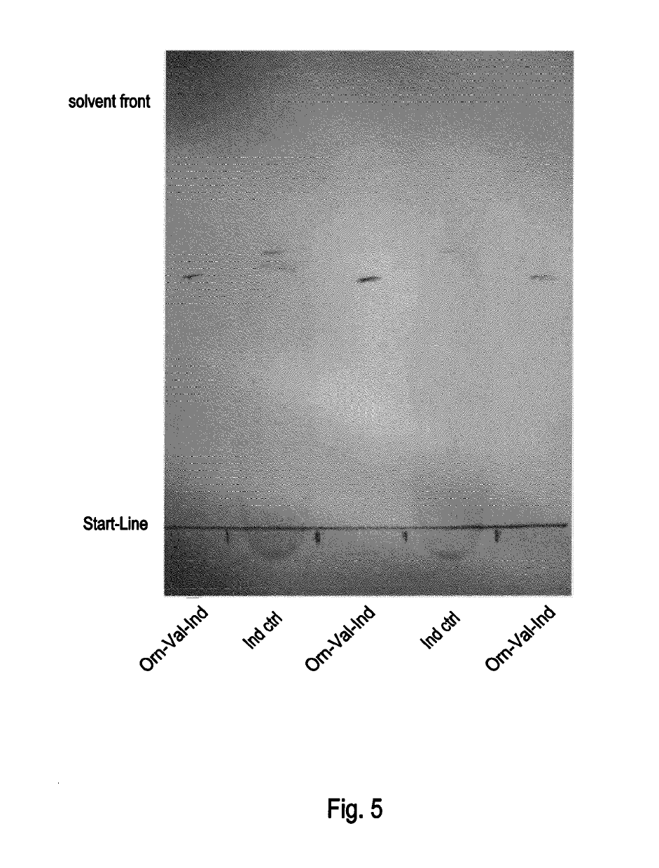

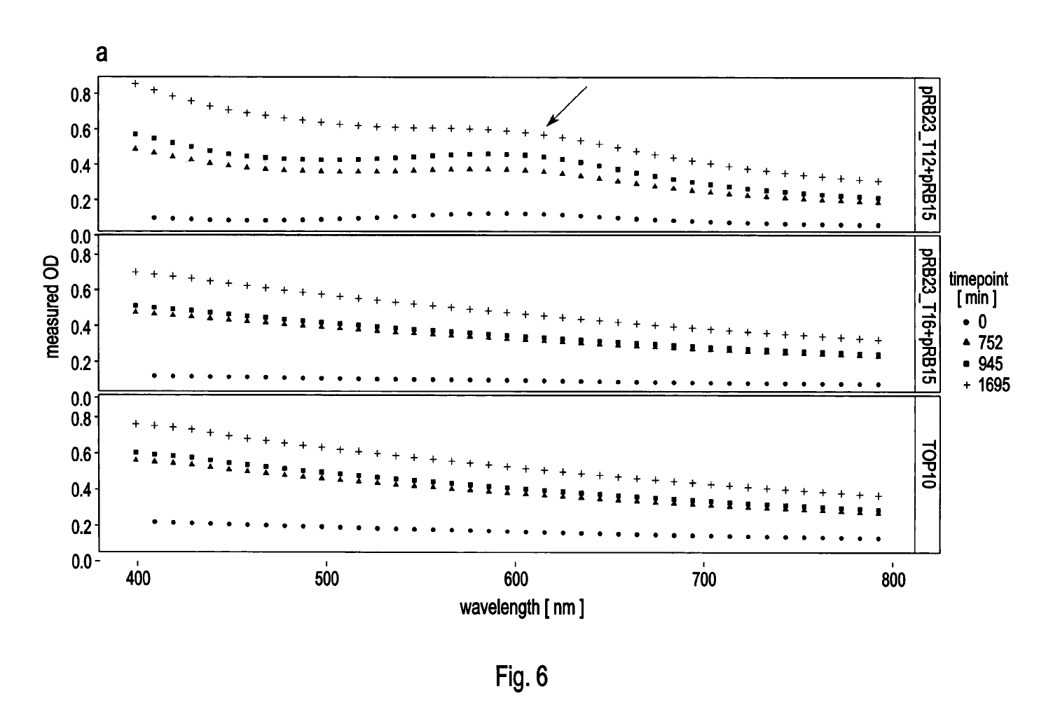

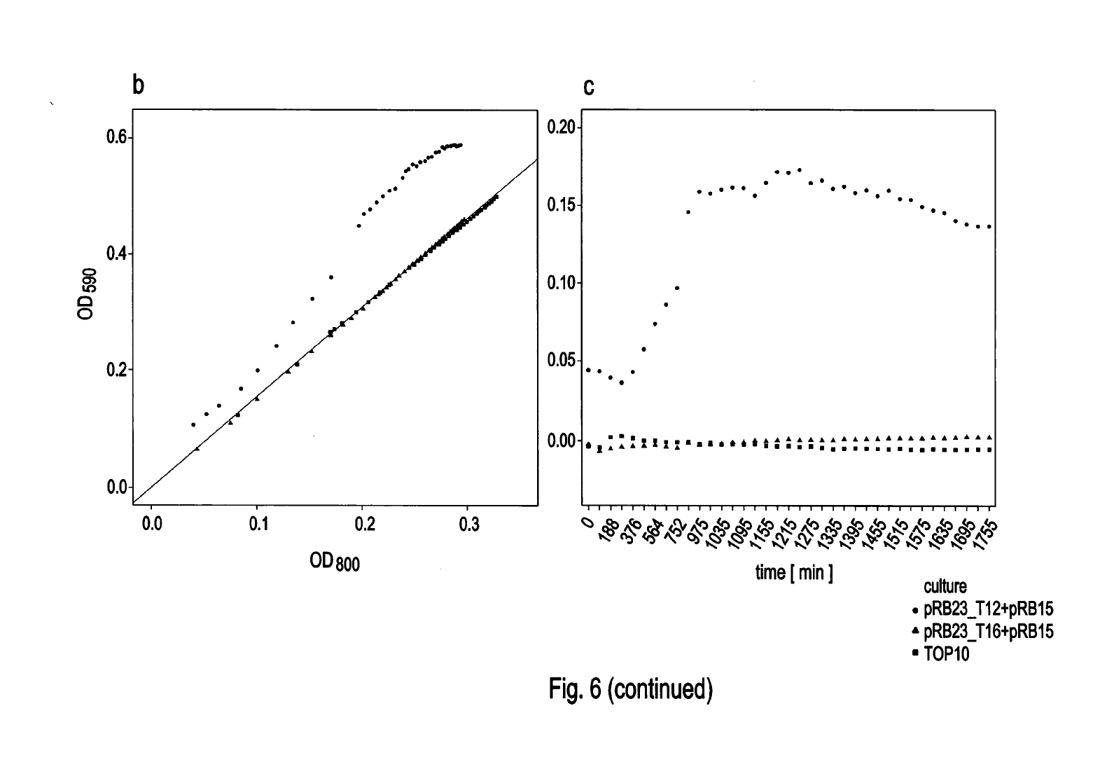

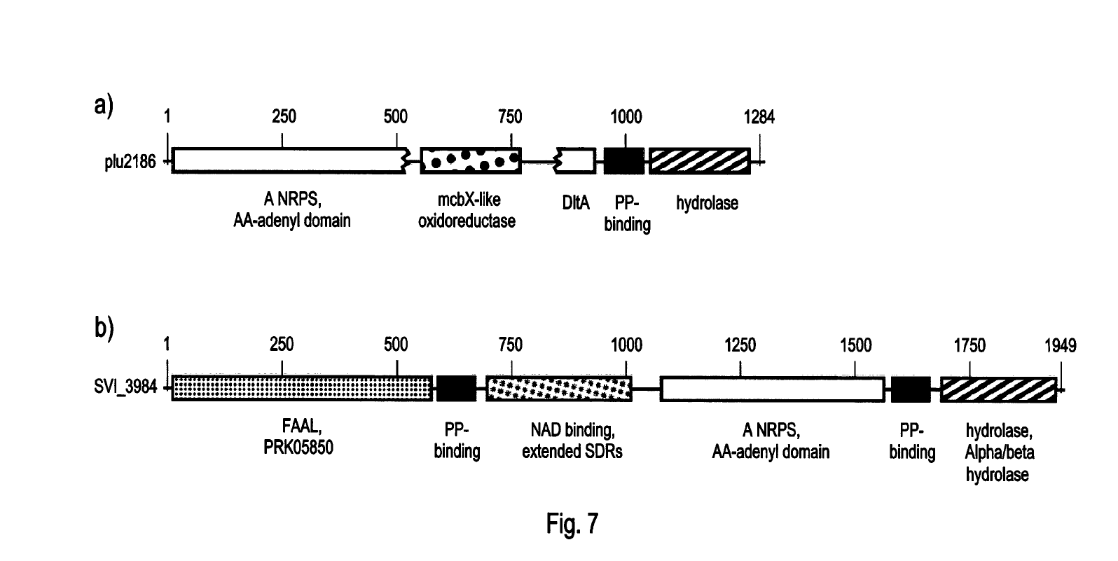

With the aforementioned protocols for purification and TLC, we were able to confirm the successful expression of an Orn-Val-Ind fusion peptide, which shows slower migration on the TLC compared to Indigoidine control (FIG. 5).

### EXAMPLE 2

### Quantitative Indigoidine Assay

### OD Measurement

We prepared a pre-culture in 96 well plates with 100 μl of LB-media with the respective antibiotics (chloramphenicol and kanamycin) and picked colonies from every positive plate from a cotransformation experiment where an indC-expression construct was transformed alongside with a PPTase-expression construct. We incubated the pre-cultures for 24 hours at 37° C. and inoculated the measurement plate with 20 ul of the pre-culture and 180 μl of LB-medium. We measured the absorbance from 400 nm to 800 nm in intervals of 10 nm for each well every 30 minutes for 30 hours at 30° C. in a Tecan infinite M200 plate reader. For the measurement, we used Greiner 96 well flat black plates with a clear lid.

### Data Analysis

Indigoidine has a maximum absorption at a wavelength about 590 nm. Since usually the cell density is measured at 600 nm we had to find another method to be able to track both the optical density of the liquid culture and the contribution of indigoidine to the absorption at 590 nm.

For the analysis we basically used the OD values at two wavelengths: The OD590 for the absorption of indigoidine and liquid culture and the OD800 as a robust wavelength to measure the cell density without the influence of the indigoidine absorption spectrum. Assuming that in a normal liquid culture without indigoidine OD590=δ*OD800 (Myers et al. (2013), Bmc Biophysics 6: 4), we used a negative control (TOP10 cotransformed with a PPTase and an unfunctional indigoidine synthetase variant) to determine δ. If we now take the OD590 of our indigoidine producing liquid cultures and subtract δ*OD800 we get the absorption of indigoidine without the background of the liquid culture. We are now able to quantitatively observe the indigoidine production of a liquid culture over time as well as the indigoidine production in relation to the cell growth when comparing the OD590 of indigoidine with the OD800 of the cells.

### EXAMPLE 3

### N,N′-Dodecylindigoidine Synthetase

In a recent study by Kobayashi et. al. (“New violet 3,3′-bipyridyl pigment purified from deep-sea microorganism Shewanella violacea DSS12.,” Extremophiles: life under extreme conditions, vol. 11 (2007):245-50), an indigoidine derivative in Shewanella violacea was characterized. It was found that this bacterium produces a pigment which has organic dodecyl chains attached to the indigoidine core structure (N,N′-dodecylindigoidine, formula (I)).

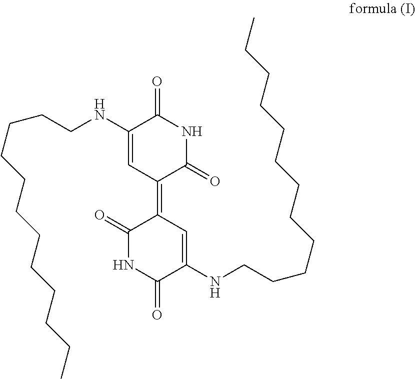 [SMILES: CCCCCCCCCCCCNC1=C/C(=C2/C=C(NCCCCCCCCCCCC)C(=O)NC2=O)C(=O)NC1=O]

However, the researchers of this study did not identify the genetic locus in the genome of Shewanella violacea or the biochemical pathway responsible for pigment synthesis. Accordingly, a bioinformatics approach was used in order to identify the genes and biochemical pathway leading to synthesis of N,N′-dodecylindigoidine (formula I) in Shewanella violacea. The Shewanella violacea genome assembly was completed in 2010 and is available under Genbank Ace. No: NC 014012.1, GI:294138771.

### A) Methods

The amino acid sequence of the indigoidine synthetase indC from Photorhabdus luminescens was used for a DELTA-BLAST query (Boratyn et al. (2012), “Domain enhanced lookup time accelerated BLAST.,” Biology direct 7:12) against a non-redundant database restricted to the organism Shewanella violacea DSS12 (taxid: 637905). The 10 most significant hits were evaluated by their query coverage and identity with the query. In order to further validate possible candidate genes we subjected the whole genome sequence of Shewanella violacea to antiSMASH 2.0 (Blin et al. (2013), “antiSMASH 2.0—a versatile platform for genome mining of secondary metabolite producers.,” Nucleic acids research, 41:W204), a web server which helps researchers to predict second metabolite pathways in microbial genomes. The identified clusters were evaluated for presence of putative NRPSs focusing on the candidate genes obtained from the previous step. For the domain prediction for indC as well as for SVI_3984, two different services were used: the NCBI Conserved Domains Database (CDD) (Marchler-Bauer et al. (2013), “CDD: conserved domains and protein three-dimensional structure.,” Nucleic acids research 41:D348) and Pfam (Finn et al. (2014), “Pfam: the protein families database.,” Nucleic acids research 42:D222). For each protein the amino acid sequence was used to query the respective tool. In order to predict the specificity of the identified A domains of SVI_3984 the tool NRPSPredictor2 (Röttig et al. (2011), “NRPSpredictor2—a web server for predicting NRPS adenylation domain specificity.,” Nucleic acids research 39:W362) was used.

B) Identification of a Candidate in the Shewanella violacea

In a BLAST search against the Shewanella violacea genome using indC (SEQ ID NO:25) as query, hypothetical protein SVI_3984 was identified as the most promising hit (69% query covery, 31% identity, E-value: 1e-124; Genbank Ace. No.: NC_014012.1 GI:294138771/SEQ ID NO: 39 (gene), Genbank Ace. No.: YP_003558733.1 gi 294142755/SEQ ID NO:38 (protein). The antiSMASH webserver identified 13 biosynthetic clusters for Shewanella violacea DSS12. Within these, only the gene SVI_3984 encodes for a putative NRPS.

FIG. 7 graphically summarizes the results of the CDD and Pfam predictions in tables 3 to 6. The indigoidine synthetase indC from Photorhabdus luminescens (SEQ ID NO:25) is a one-module NRPS with an adenylation domain with embedded oxidation domain, a PP-binding carrier domain and a thioesterase domain (Reverchon et al. (2002), “Characterization of indigoidine biosynthetic genes in Erwinia chrysanthemi and role of this blue pigment in pathogenicity.,” Journal of bacteriology 184:654; Takahashi et al. (2007), “Cloning and characterization of a Streptomyces single module type non-ribosomal peptide synthetase catalyzing a blue pigment synthesis.,” The Journal of biological chemistry 282:9073; Brachmann et al. (2012), “Triggering the production of the cryptic blue pigment indigoidine from Photorhabdus luminescens.,” Journal of biotechnology 157:96). The hypothetical protein SVI_3984 is apparently a two-module NRPS and it has the composition: adenylation domain, carrier domain, NAD binding protein, then again an adenylation and a carrier domain and finally a thioesterase domain. The NAD binding domain (PF07993) is also denoted as male sterility protein which again is associated with reductases in condensation reactions (Aarts et al. (1997), “The Arabidopsis MALE STERILITY 2 protein shares similarity with reductases in elongation/condensation complexes.,” The Plant journal: for cell and molecular biology 12:615).

The predicted domain specificities for SV13984 are shown in Table 7.

### C) Result

It is found that N,N′-dodecylindigoidine is synthesized by a 2-module NRPS encoded by gene SVI_3984 (Genbank Acc No: NC_014012.1 GI:294138771). The NRPS is composed of a C-terminal indigoidine synthetase and an N-terminal module carrying an NAD-binding domain, mediating the covalent attachment of the indigoidine pigment to the organic dodecyl chain. Preferably, SVI_3984 represents an interesting scaffold NRPS for potential fusion of NRPs or other organic molecules with NRPS-derived pigments.

TABLE 2
DNA sequences used in the Examples:

| | | SEQ ID | encoded polypeptide |
|---|---|---|---|
| Sequence name | Brief Description | NO | SED ID NO |
| indC | native indigoidine synthetase indC from Photorhabdus luminescens | 1 | |
| | laumondii TT01 | | |
| indC-ccdB | engineered and functional indC from P. luminescens where we replaced the | 2 | |
| | native T-domain with a ccdB gene which is toxic to normal E. coli cells. We | | |
| | used this variant to easily exchange T-domains avoiding any negative clones | | |
| | (cloning background). | | |
| indC-T2 | engineered and functional indC from P. luminescens where we replaced the | 3 | |
| | native T-domain with the T-domain of the bpsA indigoidine synthetase from | | |
| | Streptomyces lavendulae lavendulae ATCC11924 | | |
| indC-T8 | engineered and functional indC from P. luminescens where we replaced the | 4 | |
| | native T-domain with the T-domain of the plu2642 protein from P. | | |
| | luminescens | | |
| indC-T6 | engineered and functional indC from P. luminescens where we replaced the | 5 | |
| | native T-domain with the T-domain of the delH4 protein from Delftia | | |
| | acidovorans SPH-1 | | |
| indC-T10 | engineered and functional indC from P. luminescens where we replaced the | 6 | |
| | native T-domain with a synthetic T-domain of our own design (variant 1) | | |
| indC-T12 | engineered and functional indC from P. luminescens where we replaced the | 7 | |
| | native T-domain with a synthetic T-domain of our own design (variant 3) | | |
| indC-T13 | engineered and functional indC from P. luminescens where we replaced the | 8 | |
| | native T-domain with a synthetic T-domain of our own design (variant 4) | | |
| indC-T14 | engineered and functional indC from P. luminescens where we replaced the | 9 | |
| | native T-domain with a synthetic T-domain of our own design (variant 5) | | |
| plu2642 | gene of unknown function from P. luminescens laumondii TT01; Pfam | 10 | |
| | prediction suggests a single module NRPS with glutamine specificity and and | | |
| | an amino acid sequence being similar to other Indigoidine-synthetase | | |
| | sequences | | |
| Asn-Ind | NRPSase being a synthetase of a fusion peptide consisting of Asparagine and | 11 | 27 |
| | Indigoidine | | |
| ccdB-Ind | Construct that enables easy cloning of NRPS modules in front of Indigoidine | 12 | |
| | module through the exchange of ccdB. | | |
| Orn-Val-Ind | NRPSase synthesizing a Indigoidine-tagged Dipeptide consisting of Ornithine | 13 | 28 |
| | and Valine | | |
| Orn-Val-Val-Ind | NRPSase synthesizing a Indigoidine-tagged Tripeptide consisting of Ornithine | 14 | 29 |
| | and two Valines | | |
| Phe-Ind | NRPSase being a putative synthetase of a fusion peptide consisting of | 15 | 30 |
| | Phenylalanine and Indigoidine | | |
| Phe-Orn-Leu-Ind | NRPSase synthesizing a Indigoidine-tagged Tripeptide consisting of | 16 | 31 |
| | Phenylalanine, Ornithine and Leucine | | |
| Phe-Orn-Leu-Val-Ind | NRPSase synthesizing a Valine-Indigoidine-tagged Tripeptide consisting of | 17 | 32 |
| | Phenylalanine, Ornithine and Leucine. Valine is here used as spacer. | | |
| Pro-Leu-Ind | NRPSase synthesizing a Indigoidine-tagged Dipeptide consisting of Proline | 18 | 33 |
| | and Leucine | | |
| Pro-Leu-Val-Ind | NRPSase synthesizing a Valine-Indigoidine-tagged Dipeptide consisting of | 19 | 34 |
| | Proline and Leucine. Valine is here used as spacer. | | |
| TycA | Entire gene from the Tyrocidine-cluster. TycA is one module consisting of an | 20 | |
| | A-, T- and E-domain | | |
| TycB | Entire gene from the Tyrocidine-cluster. TycB consists of 3 modules. | 21 | |
| TycC | Entire gene from the Tyrocidine-cluster. TycC consists of 6 modules, in the | 22 | |
| | final module of TycC6, Tyrocidine is cyclized | | |
| Val-Ind | NRPSase being a synthetase of a fusion peptide consisting of Valine and | 23 | 35 |
| | Indigoidine. Due to its sterical advantages, Valine may be used as a spacer | | |
| | for other tags. | | |
| Val-Val-Ind | NRPSase synthesizing a Indigoidine-tagged Dipeptide consisting of two | 24 | 36 |
| | Valine-monomers. | | |
| C(TycC2)-Ind | Minimal construct - requires addition of at least one A and T domain | 26 | 37 |

TABLE 3
Domain order predicted by NCBI CDD for indC.

| Name | CDD Accession | Description | Interval | E-value |
|---|---|---|---|---|
| A NRPS | cd05930 | adenylation domain of NRPS | 41-486 | 1.14e−145 |
| AA-adenyl-dom | TIGR01733 | amino acid adenylation domain | 75-460 | 3.01e−121 |
| mcbC-like-oxidoreductase | cd02142 | family of oxydase domain of NRPS and other | 564-759 | 2.20e−26 |
| DltA | cd05945 | D-alanine:D-alanyl carrier protein ligase | 889-928 | 1.45e−09 |
| PP-binding | pfam00550 | Phosphopantetheine attachment site | 953-1016 | 3.52e−12 |
| Hydrolase-4 super family | cl19140 | putative lysophospholipase | 1037-1146 | 1.08e−24 |

TABLE 4
Domain order predicted by Pfam for indC.

| Name | Description | Pfam Accession | Envelope Interval | E-value |
|---|---|---|---|---|
| AMP-binding | AMP-binding enzyme | PF00501.23 | 28-437 | 1.5e−94 |
| PP-binding | Phosphopantetheine attachment site | PF00550.20 | 953-1016 | 1.6e−12 |
| Thioesterase | Thioesterase domain | PF00975.15 | 1036-1186 | 2.5e−23 |

TABLE 5
Domain order predicted by NCBI CDD for SVI3984.

| Name | CDD Accession | Description | Interval | E-value |
|---|---|---|---|---|
| PRK05850 | PRK05850 | acyl-CoA synthetase | 13-567 | 1.28e−115 |
| FAAL | cd05931 | Fatty acyl-AMP ligase (FAAL) | 20-561 | 0e+00 |
| PP-binding | pfam00550 | Phosphopantetheine attachment site | 596-650 | 5.19e−08 |
| NAD-binding-4 | pfam07993 | Male sterility protein | 687-923 | 7.69e−40 |
| SDR e1 | cd05235 | extended (e) SDRs, subgroup 1 | 686-969 | 2.01e−50 |
| A-NRPS | cd05930 | adenylation domain of NRPS | 1089-1567 | 0e+00 |
| AA-adenyl-dom | TIGR01733 | amino acid adenylation domain | 1102-1504 | 1.74e−150 |
| PP-binding | pfam00550 | Phosphopantetheine attachment site | 1590-1653 | 1.28e−11 |
| Hydrolase-4 super family | cl19140 | putative lysophospholipase | 1682-1940 | 2.63e−23 |
| Abhydrolase-6 | pfam12697 | Alpha/beta hydrolase family | 1683-1804 | 1.06e−06 |

TABLE 6
Domain order predicted by Pfam for SVI3984.

| Name | Description | Pfam Accession | Envelope Interval | E-value |
|---|---|---|---|---|
| AMP-binding | AMP-binding enzyme | PF00501.23 | 18-456 | 8.8e−87 |
| PP-binding | Phosphopantetheine attachment site | PF00550.20 | 595-652 | 3.2e−06 |
| NAD-binding-4 | Male sterility protein | PF07993.7 | 687-923 | 2.8e−42 |
| AMP-binding | AMP-binding enzyme | PF00501.23 | 1081-1480 | 8.7e−103 |
| AMP-binding-C | AMP-binding enzyme C-terminal domain | PF13193.1 | 1488-1561 | 2.7e−11 |
| PP-binding | Phosphopantetheine attachment site | PF00550.20 | 1590-1653 | 6.6e−13 |
| Thioesterase | Thioesterase domain | PF00975.15 | 1681-1938 | 6.6e−20 |

TABLE 7
Predicted A domain specificity for SVI3984

| Name | Interval | Prediction | Score | Precision |
|---|---|---|---|---|
| A domain 1 | 195-350 | hydrophobic-aliphatic | 0.566730 | 0.974 |
| A domain 2 | 1239-1380 | asp,asn,glu,gln,aad | 0.773129 | 0.969 |

(aad: alpha-amino-adipic acid)

1-15. (canceled)

16. A polypeptide or polypeptide complex, comprising at least one non-ribosomal peptide synthesis (NRPS) amino acid module functionally connected to at least one pigment module.

17. The polypeptide or polypeptide complex of claim 16, wherein the pigment module is an NRPS pigment module.

18. The polypeptide or polypeptide complex of claim 17, comprising the C domain of the at least one NRPS amino acid module and the at least one NRPS pigment module as a fusion polypeptide.

19. The polypeptide or polypeptide complex of claim 16, wherein the pigment module is an indigoidine synthetase.

20. A labeled oligopeptide comprising a non-naturally attached NRPS pigment or/and polyketide pigment.

21. The labeled oligopeptide of claim 20, wherein the pigment is indigoidine.

22. A polynucleotide encoding a fusion polypeptide according to claim 18.

23. A vector, preferably an expression vector, comprising the polynucleotide according to claim 22.

24. An in vitro method of producing a labeled oligopeptide, comprising:
a) incubating a polypeptide or polypeptide complex according to claim 16 with appropriate amino acid substrates,
b) thereby producing a labeled oligopeptide.

25. An in vivo method of producing a labeled oligopeptide, comprising:
a) incubating a host cell comprising the polypeptide or polypeptide complex according to claim 16,
b) thereby producing a labeled oligopeptide.

26. A method for optimizing in vivo production of a labeled oligopeptide, comprising
a) incubating a host cell comprising a variant of a polypeptide or polypeptide complex according to claim 16 under conditions suitable for production of said labeled oligopeptide,
b) comparing the amount of labeled oligopeptide produced to the amount produced by a host cell comprising an unmodified polypeptide or polypeptide complex according to claim 1, and, thereby
c) optimizing in vivo production of a labeled oligopeptide.

27. A method for optimizing in vitro production of a labeled oligopeptide, comprising
a) incubating a variant of a polypeptide or polypeptide complex according to claim 16 under conditions suitable for production of said labeled oligopeptide,
b) comparing the amount of labeled oligopeptide produced to the amount produced by the unmodified polypeptide or polypeptide complex, and, thereby
c) optimizing in vitro production of a labeled oligopeptide.
